(19) United States
(12) Patent Application Publication
(10) Pub. No.: US 2023/0183746 A1
(43) Pub. Date: Jun. 15, 2023

(54) COMPOSITIONS FOR TRANSFER OF CARGO TO CELLS

(71) Applicant: Sixfold Bioscience Ltd., London (GB)

(72) Inventors: George William Foot, Dorchester (GB); Anna Perdrix Rosell, London (GB); Zuzanna Aleksandra Brzosko, London (GB); Catherina Gasser, London (GB)

(21) Appl. No.: 17/638,644

(22) PCT Filed: Aug. 28, 2020
(86) PCT No.: PCT/IB2020/001023

§ 371 Date: Feb. 25, 2022

Related U.S. Application Data
(60) Provisional application No. 62/894,390, filed on Aug. 30, 2019.

(51) Int. Cl. C12N 15/87; C12N 15/113

(52) U.S. Cl. CPC C12N 15/87; C12N 15/1137; C12N 2310/14; C12N 2310/322; C12N 2310/351

(57) ABSTRACT

The invention provides compositions containing cargo molecules attached to elements that improve the function of the cargo molecules in the body of a subject. The compositions are useful for therapeutic and diagnostic purposes.

### CROSS-REFERENCE TO RELATED APPLICATION

This application is a §371 national stage entry of PCT International Application No. PCT/IB2020/001023, filed Aug. 28, 2020, which claims the benefit of, and priority to, U.S. Provisional Pat. Application No. 62/894,390, filed Aug. 30, 2019, the contents of each of which are incorporated herein by reference.

### FIELD OF THE INVENTION

The invention relates generally to therapeutic compositions that include nucleic acid nanoparticles for delivery of cargo and methods of using the same.

### SEQUENCE LISTING

The present application incorporates by reference a Sequence Listing in electronic format. The sequence listing is provided as an ASCII text file entitled “SIX-002-01US-Sequence-Listing.txt”, which was created on Aug. 16, 2022 and is 32.4 kilobytes size.

### BACKGROUND

A broad problem that hampers a wide range of therapeutic and diagnostic procedures is transferring information into cells. For therapeutics, a common challenge is delivery of an agent, such as a functional molecule or nucleic acid that carries genetic information, into diseased cells to target them for destruction. Alternatively, the problem may manifest in the inefficiency in provide a molecule to healthy cells to stimulate their activity. In a diagnostic context, the problem often centers around the difficulty of delivering to a marker that will identify cells as diseased or reveal information about their functional properties. Due to such technical barriers, many of the insights into the mechanisms of many human conditions gained from research in the laboratory have not been translated into clinical advances that allow better diagnosis and treatment of such conditions.

### SUMMARY

The invention provides compositions for improved transfer of cargo, such as RNA molecules, into cells. The invention includes a variety of compositions that have improvements in one or more features essential for delivery of cargo to target cells and functionality of the cargo upon arrival. For example, compositions of the invention display increased stability of cargo en route to target cells, increased cell specificity, better cellular internalization and delivery of cargo to proper intracellular destinations, and reduced activity of cargo in off-target cells. Consequently, the compositions allow superior transfer of genetic information contained in RNA molecules and other types of cargo into specific cell types.

The compositions of the invention are useful in a broad range of medical and biological applications. Due to their ability to deliver genetic information, certain compositions may function as therapeutics in the treatment of genetically-driven diseases, such as cancer, autoimmune diseases, inflammatory disorders, and infections. Because compositions of the invention allow activity of agents to be triggered in targeted cells but blocked in other cells, they are well-suited for delivery of potentially hazardous cargo that are needed to treat such serious diseases. The compositions may also serve as diagnostic tools that identify genetically aberrant cells in a subject and thus allow early detection of genetic diseases and conditions.

In an aspect, the invention provides compositions that include a RNA molecule in a compacted configuration and a nucleic acid nanoparticle attached to the RNA molecule. The RNA molecule may be mRNA molecule, a lnRNA molecule, a miRNA molecule, a siRNA molecule, or a shRNA molecule.

The composition may include multiple RNA molecules. For example, the composition may contain 1, 2, 3, 4, 5, 6,7, 8, 9,10, 11,12, or more RNA molecules. Each of the RNA molecules may be attached to the nucleic acid nanoparticle.

Each mRNA molecule may independently have a structural element that maintains the mRNA molecule in a compacted configuration. The structural element may be a codon variant, a structural motif, nucleotides that form an intramolecular base pair within a RNA molecule, nucleotides that form an intermolecular base pair between RNA molecules, a 5′ cap comprising a non-naturally-occurring nucleotide, or an elongated 3′ polyadenylated tail.

The composition may contain different mRNA molecules that encode different polypeptides, and the different polypeptides may interact to form a functional protein.

The mRNA molecule and the nucleic acid nanoparticle may be attached via a linker. The linker may reversibly link the mRNA molecule to the nucleic acid nanoparticle. The linker may be an azide-alkyne bond, a biotin-streptavidin linkage, a disulfide bond, an enzymatically-cleavable linkage, an acid-labile linkage, a ribozyme linkage, or a nucleotide base pair.

The composition may include a packing component that maintains the RNA molecule in a compacted configuration. The composition may include 1, 2, 3, 4, 5, 6, 7, 8, 9, 10, 11, 12, or more packing components. The packing component may be a nucleic acid, peptide, polypeptide, protein, lipid or small (i.e., having a molecular weight of < 2000 Da) organic molecule. The nucleic acid may be RNA, DNA, or a RNA/DNA hybrid. The nucleic acid may be single-stranded or double-stranded, or it may include both single-stranded and double-stranded portions. The peptide, polypeptide, or protein may include a histone. The packing component may include two or more molecules that bind to each other. The binding of the two or more molecules may be regulatable.

The nucleic acid nanoparticle may be a RNA nanoparticle, a DNA nanoparticle, or a particle that contains both RNA and DNA.

In another aspect, the invention provides compositions that include a cargo molecule and an element that is linked to the cargo molecule and functionalize to promote a biological activity of the cargo molecule in a subject.

The cargo molecule may be, or include, a RNA molecule, DNA molecule, peptide, polypeptide, protein, or any combination thereof. The RNA molecule may be mRNA molecule, a lnRNA molecule, a miRNA molecule, a siRNA molecule, or a shRNA molecule. The cargo molecule may be, or may encode, a CRISPR component. The cargo molecule may be, or may encode, a chimeric antigen receptor.

The element may regulate expression of a mRNA cargo molecule in a cell in a subject.

The element may inhibit degradation of a cargo RNA molecule, such as a mRNA molecule. The element may be or include a packing component, such as any of those described above, a nuclease inhibitor, a lnRNA molecule, a miRNA molecule, an siRNA molecule, or a shRNA molecule. The nuclease inhibitor may be an RNase inhibitor. The RNase inhibitor may be barstar or a polycation.

The element may enhance translation of a mRNA cargo molecule. The element may include or be a riboswitch, a ribosome recruitment sequence, or an aptamer that targets the mRNA molecule to a cytoplasm of a cell. The ribosome recruitment sequence may be an internal ribosome entry site.

The element may permit expression of the mRNA molecule in a first cell type and inhibit expression of a mRNA cargo molecule in a second cell type.

The element may inhibit translation of the mRNA molecule a cell type. The element may promote degradation of the mRNA molecule in a cell type. The component may include or be a target sequence in the mRNA molecule. The target sequence may be recognized by a nuclease, such as Dicer. The target sequence may be complementary to a RNA molecule expressed in the second cell type, such as a miRNA molecule, shRNA molecule, or shRNA molecule. The element may include or be a RNA molecule that recognizes a target sequence in the mRNA molecule, such as a miRNA molecule, shRNA molecule, or siRNA molecule. The element may include or be another mRNA molecule that encodes a nuclease that degrades the first mRNA molecule.

The element may promote internalization of the cargo molecule into a cell in the subject. The element may include a lipophilic moiety. The lipophilic moiety may be a fluoro group or a methoxyethyl group. The element may include a positively-charged moiety. The positively-charged moiety may be a cell-penetrating peptide or a guanidyl group.

The element may promote intracellular availability of the cargo molecule into a cell in the subj ect.

The element may promote delivery of the cargo molecule to an intracellular site within the cell of the subject. The intracellular site may be the nucleus, cytoplasm, ribosome, endoplasmic reticulum or mitochondria.

The element may be linked to the cargo molecule via a nucleic acid nanoparticle.

The element may promote degradation of the nucleic acid nanoparticle in a cell type. The element may be or include a target sequence, a mRNA molecule, a miRNA molecule, a shRNA molecule, or a siRNA molecule. The target sequence may be in a nucleic acid in the nanoparticle. The target sequence may be recognized by a nuclease, such as Dicer. The target sequence may be complementary to a RNA molecule expressed in a cell type, such as a miRNA molecule, shRNA molecule, or shRNA molecule. The element may be or include a RNA molecule that recognizes a target sequence in a nucleic acid in the nanoparticles, such as a miRNA molecule, shRNA molecule, or siRNA molecule. The element may be or include a mRNA molecule that encodes a nuclease that degrades a nucleic acid in the nanoparticle.

The element may be conjugated to a nucleic acid in the nucleic acid nanoparticle via linkage to a 2′ position of a sugar in the nucleic acid, a base in the nucleic acid, or the phosphorus-containing backbone of the nucleic acid. The nucleic acid nanoparticle may include a nucleic acid having one or more of the following linkages: a 3-(2-nitrophenyl)-propyl phosphoramidite linkage, a 3-phenylpropyl phosphoramidite linkage, a alkyl phosphorothioate linkage, a aminobutyl phosphoramidite linkage, a aryl phosphorothioate linkage, a dimethylamino phopsphoramidite linkage, a guanidinobutylphosphoramidate linkage, and a phosphorothioate linkage.

The nucleic acid nanoparticle may be an RNA nanoparticle, a DNA nanoparticle, or a particle that contains both RNA and DNA.

In another aspect, the invention provides compositions that include a cargo molecule linked to an element that promotes escape of the cargo molecule from an endosome within a cell of a subject.

The element that promotes endosomal escape may have a pKₐ of from about 5.0 to about 7.0.

The element that promotes endosomal escape may include a hydrophobic component. The hydrophobic component may be a cholesterol lipid.

The element that promotes endosomal escape may be positively-charged at a pH of about 7.0. The element may include a guanidyl group.

The element that promotes endosomal escape may include a peptide.

The element that promotes endosomal escape may be linked to the cargo molecule via a nanoparticle. The element may be conjugated to a nucleic acid in the nanoparticle. The element may be conjugated to a 2′ position of a nucleic acid in the nucleic acid nanoparticle. The element may be conjugated to a base of a nucleic acid in the nucleic acid nanoparticle. The element may be conjugated to a phosphorus-containing linkage of nucleotides in the nucleic acid nanoparticle. The phosphorus-containing linkage may be a 3-(2-nitrophenyl)-propyl phosphoramidite linkage, a 3-phenylpropyl phosphoramidite linkage, a alkyl phosphorothioate linkage, a aminobutyl phosphoramidite linkage, a aryl phosphorothioate linkage, a dimethylamino phopsphoramidite linkage, a guanidinobutylphosphoramidate linkage, or a phosphorothioate linkage.

The nucleic acid nanoparticle may include a component that includes a hydrophobic moiety, a hydrophilic moiety, and a nucleotide attachment moiety. The hydrophilic moiety may include an amine. The hydrophilic moiety may be spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(Hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, pyrrole imidazoles, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, or a β-aminoester. The nucleotide attachment moiety may be cysteine.

The element may promote endosomal escape of the cargo molecule in a receptor independent-manner. The element may bind to a receptor in the cell. The element may include folate, TfR-T₁₂, or a hemagglutinin peptide.

The cargo molecule may be, or include, a RNA molecule, DNA molecule, peptide, polypeptide, protein, a small organic molecule, or any combination thereof. The RNA molecule may be mRNA molecule, a lnRNA molecule, a miRNA molecule, a siRNA molecule, or a shRNA molecule. The cargo molecule may be, or may encode, a CRISPR component. The cargo molecule may be, or may encode, a chimeric antigen receptor.

The nucleic acid nanoparticle may be an RNA nanoparticle, a DNA nanoparticle, or a particle that contains both RNA and DNA.

In another aspect, the invention provides compositions that include a cargo molecule linked to an element that binds or repels a protein in plasma of a subject.

The element may bind a plasma protein. The element may repel a plasma protein. The element may bind a first plasma protein and repel a second plasma protein.

The protein in the plasma may be albumin, fibrinogen, fibronectin, haptoglobin, immunoglobulin, α-1-acidglycoprotein, α1-antitrypsin, α-2-macroglobulin, or α-thrombin.

The element may be an aptamer.

The element that binds or repels a plasma protein may be linked to the cargo molecule via a nanoparticle. The nucleic acid nanoparticle may include a nucleic acid having a phosphorus-containing linkage of nucleotides. The phosphorus-containing linkage may be a 3-(2-nitrophenyl)-propyl phosphoramidite linkage, a 3-phenylpropyl phosphoramidite linkage, a alkyl phosphorothioate linkage, a aminobutyl phosphoramidite linkage, a aryl phosphorothioate linkage, a dimethylamino phosphoramidite linkage, a guanidinobutylphosphoramidate linkage, or a phosphorothioate linkage.

The element may be conjugated to a nucleic acid in the nanoparticle. The element may be conjugated to a 2′ position of a nucleic acid in the nucleic acid nanoparticle. The element may be conjugated to a base of a nucleic acid in the nucleic acid nanoparticle. The element may be conjugated to a phosphorus-containing linkage of nucleotides in the nucleic acid nanoparticle, such as one of those described above.

The nucleic acid nanoparticle may promote internalization of the cargo molecule into a cell in a subject. The nucleic acid nanoparticle may include a lipophilic moiety. The lipophilic moiety may be a fluoro group or a methoxyethyl group. The nucleic acid nanoparticle may include a positively-charged moiety. The positively-charged moiety may be a cell-penetrating peptide, guanidyl group, or modified nucleotide.

The nucleic acid nanoparticle may include a component that promotes internalization of the cargo molecule into a cell in the subject. The component may be conjugated to a nucleic acid in the nucleic acid nanoparticle via linkage to either a 2′ position of a sugar in the nucleic acid or a base in the nucleic acid.

### BRIEF DESCRIPTION OF THE DRAWINGS

FIG. 1 is a schematic of a nucleic acid nanoparticle according to an embodiment of the invention.

FIG. 2 is a schematic of a composition that includes a nucleic acid nanoparticle, linker, and mRNA molecule according to an embodiment of the invention.

FIG. 3 shows formation of a disulfide linkage that can be used to attach a cargo molecule to a nucleic acid nanoparticle according to an embodiment of the invention.

FIG. 4 shows an example of a linker that can be used to attach a cargo molecule to a nucleic acid nanoparticle according to an embodiment of the invention.

FIG. 5 shows an example of a linker that can be used to attach a cargo molecule to a nucleic acid nanoparticle according to an embodiment of the invention.

FIG. 6 shows formation of an azidomethyl-methylmaleic anhydride that can be used to attach a cargo molecule to a nucleic acid nanoparticle according to an embodiment of the invention.

FIG. 7 is a schematic showing cleavage of a composition according to an embodiment of the invention.

FIG. 8 shows a method of using RNA origami to produce compacted mRNA molecules.

FIG. 9 shows examples of mRNA origami folding principles.

FIG. 10 shows the use of “pins” to hold mRNA in a compacted configuration.

FIG. 11 shows mRNA molecules that self-assemble into nanoparticles.

FIG. 12 is a schematic of composition according to an embodiment of the invention.

FIG. 13 is a schematic of a composition according to an embodiment of the invention.

FIG. 14 is a schematic of a composition according to an embodiment of the invention.

FIG. 15 is a schematic of a composition according to an embodiment of the invention.

FIG. 16 is a schematic of a composition according to an embodiment of the invention.

FIG. 17 show sites of modification of a nucleotide in nanoparticles according to an embodiment of the invention.

FIG. 18 is graph showing endosomal escape using various compounds alone or in combination.

FIG. 19 is graph showing endosomal escape using various compounds alone or in combination.

FIG. 20 is a graph showing endosomal escape using various compounds.

FIG. 21 is a graph showing endosomal escape using various compounds.

FIG. 22 is a graph showing toxicity effects of endosomal escape compounds.

FIG. 23 is a schematic of a composition according to an embodiment of the invention.

FIG. 24 is a schematic of a composition according to an embodiment of the invention.

FIG. 25 is a schematic of a composition according to an embodiment of the invention.

FIG. 26 is a schematic of a composition according to an embodiment of the invention.

FIG. 27 is a schematic of a composition according to an embodiment of the invention.

FIG. 28 is a schematic of a composition according to an embodiment of the invention.

FIG. 29 is a schematic showing activity of composition according to an embodiment of the invention.

FIG. 30 is a graph showing the effect of a protein corona on cellular uptake of RNA nanoparticles.

FIG. 31 is a schematic outlining the core construct SQ1-0000-001 used in an embodiment of the invention.

FIG. 32 is a schematic showing nucleotide base pairing in core construct SQ1-0000-001.

FIG. 33 is a graph showing dynamic light scattering of the SQ1-0000-001 core construct prior to modification.

FIG. 34 shows the results of native PAGE of the assembled SQ1-1210-001 construct with Cy5, the E07min aptamer (A-1.1), endosomal escape-mediating peptide (P-1.1) and 2 x PLK1 siRNAs (S-1.1; S-2.1).

FIG. 35 is a schematic overview over two PLK1 siRNA target sites and their respective siRNA sequences, S-5.0-S-6.0 and S-1.0-S-2.0.

FIG. 36 is a graph showing the knockdown efficacy of three different siRNA designs 72 hours after transfection of HeLa cells with 20 nM siRNA, benchmarked against the commercial SilencerSELECT siRNA (AMB).

FIG. 37 is a graphic visualization of different canonical (21-bp duplexes with 2-nt 3′ overhangs) and Dicer substrate siRNA designs (25-nt sense and 27-nt antisense strand) that were used in embodiments of the invention.

FIG. 38 shows the results of an electrophoretic mobility shift assay to verify siRNA strand annealing.

FIG. 39 is graph showing the potency of siRNAs with alkyne-modified sense strands 72 h after transfection of HeLa cells with 20 nM siRNA.

FIG. 40 is graph showing the potency of siRNAs with thiol-modified sense strands 72 h after transfection of HeLa cells with 20 nM siRNA.

FIG. 41 is a graphic summary of the positioning of 2′-sugar modifications on PLK1 siRNA S-1.0-S-2.0 that were tested for the ability to increase the in vitro stability and potency of PLK1 siRNA.

FIG. 42 is a graph of PLK1 expression in transfected MDA-MB-231 cells.

FIG. 43 shows the results of non-denaturing PAGE to verify that sense and antisense strand hybridisation by non-denaturing PAGE.

FIG. 44 shows the results of non-denaturing PAGE to demonstrate serum stability of siRNAs. siRNA duplexes were incubated for 24 h at 37° C. in 50% human serum.

FIG. 45 is a schematic showing Reversible thiol-disulfide exchange reaction used to functionalize nanoparticle core strands with siRNA.

FIG. 46 shows the results of non-denaturing PAGE demonstrating the reversible self-dimerization of 5′ thiol-modified RNA strands.

FIG. 47 is a schematic of a coupling reaction of an azide-functionalized oligo with a 2′O-modified alkyne moiety within an internal position on the second oligo use to insert CuAAC at an internal position on an RNA strand.

FIG. 48 shows the structure of a nucleotide bearing the alkyne group within the RNA strand.

FIG. 49 shows the results a denaturing PAGE following coupling reactions.

FIG. 50 is a schematic showing the coupling of an endosomal escape peptide to RNA via the NHS-PEG6-Maleimide linker.

FIG. 51 shows the results of denaturing PAGE of a linear peptide conjugated to RNA.

FIG. 52 shows the results of denaturing PAGE of a branched peptide conjugated to RNA.

FIG. 53 is a schematic of disulphide linkage using NHS-PEG₆-Maleimide.

FIG. 54 is schematic of TCEP reduction of RNA modified with thiol-modifier C6 S-S at the 5′ position.

FIG. 55 shows results of denaturing PAGE following first coupling reaction.

FIG. 56 shows results of denaturing PAGE following second coupling reaction.

FIG. 57 shows the structure of a DBCO-modified nucleotide at the 5′ end of an RNA strand.

FIG. 58 shows the structure of an azide-modified nucleotide at the 5′ end of an RNA strand.

FIG. 59 shows the results of denaturing PAGE following the coupling reaction using aqueous conditions.

FIG. 60 shows the results of denaturing PAGE following the coupling reaction using TEAA/MeCN conditions.

FIG. 61 shows the results of native 6% PAGE following biotin labelling.

FIG. 62 shows graphs of uptake of functionalized SFX1 and SFX2 aptamers analysed by flow cytometry. SXF1 aptamer (E07 min; A-1.0) is more specifically uptaken than SXF2 (CL4 aptamer) by EGFRvIII overexpressing cells.

FIG. 63 is graph showing uptake of nucleic acid nanoparticles by EGFR expressing cells.

FIG. 64 is a schematic of a Cy5-labelled construct with the most optimal SXF1 aptamer annealed through complementary base-pairing (SQ1-1000-001).

FIG. 65 shows FACS plots showing the enhancement of Cy5-labelled nanoparticle uptake by the SXF1 aptamer, specifically in cells overexpressing EGFRvIII.

FIG. 66 is graph of fluorescence uptake showing the enhancement of Cy5-labelled nanoparticle uptake by the SXF1 aptamer, specifically in cells overexpressing EGFRvIII.

FIG. 67 shows graphs of cell viability following uptake of PLK1-targeting siRNA in cancer cells.

FIG. 68 shows graphs of PLK1 mRNA and protein expression following uptake of PLK1-targeting siRNA.

FIG. 69 shows results of western blotting for PLK1 following uptake of PLK1-targeting siRNA.

FIG. 70 is a graph PLK1 mRNA expression following treatment with siRNA attached to an RNA scaffold..

FIG. 71 shows microscopic images of cells following treatment with aptamers.

FIG. 72 shows microscopic images of cells following treatment with aptamers.

FIG. 73 is a graph of siRNA released in response to glutathione.

FIG. 74 is a reverse-phase (RP) HPLC trace of GFWFG. HPLC was from 100% H2O to 100% MeCN in 15 min.

FIG. 75 is a time-of-flight mass spectrometry positive electrospray ionization (TOF MS ES+) graph of GFWFG.

FIG. 76 is a RP HPLC trace of maleimide-modified GFWFG.

FIG. 77 is a RP HPLC trace of branched GFWFG.

FIG. 78 is a TOF MS ES+ graph of branched GFWFG.

FIG. 79 is a RP HPLC trace of PEG-maleimide branched GFWFG.

FIG. 80 is a TOF MS ES+ graph of branched GFWFG.

FIG. 81 is a TOF MS ES+ graph of branched GFWFG post AMA-deprotection.

FIG. 82 shows the results of denaturing PAGE of the thiol-RNA vs. the peptide-conjugated RNA.

FIG. 83 is a RP HPLC trace of the conjugation reaction mixture.

FIG. 84 shows the results of denaturing PAGE (15%) following solid-phase peptide conjugation of thiol-functionalized RNA to maleimide-containing peptides.

FIG. 85 is a graph of hemolytic activity of a library of potential endosomal escape compounds.

FIG. 86 is a graph of hemolytic activity of most-promising endosomal escape compounds (branched GFWFG peptides with ethylene diamine linker).

FIG. 87 shows results of native PAGE (6%) to determine protein binding to RNA nanoparticles.

FIG. 88 shows results of native agarose (2.5%) gel to determine protein binding to RNA nanoparticles.

### DETAILED DESCRIPTION

The invention provides a broad range of compositions that allow delivery of cargo to cells. Examples of cargo include mRNA molecules that allow expression of exogenous polypeptides in target cells, other types of RNA molecules that permit regulation of gene expression in target cells, and other types of therapeutic or diagnostic agents. The compositions of the invention may modify the cell-specificity, cell internalization potential, and therapeutic efficacy of biological molecules and reduce off-target effects.

### Nanoparticles, Including Nucleic Acid Nanoparticles

In certain embodiments, compositions of the invention include nanoparticles. As used herein, “nanoparticle” refers to particles having dimensions that are measured on the nanometer scale. For example, a nanoparticle may have a diameter, length, width, or depth of from 1 to 1000 nm.

Any type of nanoparticle suitable for administration to a human may be used. The nanoparticle may be composed of nucleic acids, lipids, carbohydrates, proteins, polymers, silica, metals, viral components, or any combination thereof. Nucleic acid nanoparticles may be formed from RNA, DNA, or a combination of RNA and DNA. Various types of nucleic acid nanoparticles that serve as structural scaffolds have been described. Nanoparticles for delivery of cargo in biological systems are known in the art and described in, for example, U.S. Pat. No. 8,058,069; Shin, H, et al., Recent Advances in RNA Therapeutics and RNA Delivery Systems Based on Nanoparticles, Adv. Therap.2018,1, 1800065, DOI: 10.1002/adtp.201800065; Senel and Buyukkoroglu, Applications of Lipidic and Polymeric Nanoparticles for siRNA Delivery, Intech Open 2019, DOI: dx.doi.org/10.5772/intechopen.86920; and Moss, et al., Lipid Nanoparticles for Delivery of Therapeutic RNA Oligonucleotides, Mol. Pharmaceutics 2019, 16, 6, 2265-2277, doi.org/10.1021/acs.molpharmaceut.8b01290, the contents of which are incorporated herein by reference.

RNA nanoparticles are formed from the ordered arrangement of individual RNA molecules having defined secondary structures. RNA molecules form a variety of structural motifs, such as pseudoknots, kissing hairpins, and hairpin loops, that affect both the geometry of the molecule and its ability to form stable interactions with other RNA molecules via base pairing. Typically, individual RNA molecules have double-stranded regions that result from intramolecular base pairing and single-stranded regions that can for base pairs with other RNA molecules or can otherwise bind to other types of molecules.

Various RNA nanostructures having ordered two-dimensional or three-dimensional structures are known, including, for example and without limitation, nanoarrays, nanocages, nanocubes, nanoprisms, nanorings, nanoscaffolds, and nanotubes. Nanorings may be symmetrical structures that include 3, 4, 5, 6, 7, 8, or more RNA molecules arrayed around an axis. Thus, nanorings may be trimers, tetramers, pentamers, hexamers, heptamers, oxamers, or higher-numbered polymers. Nanorings may be circular, triangular, square, pentagonal, hexagonal, heptagonal, octagonal, or otherwise polygonal in shape. Other types of RNA nanoparticles, such as sheets, cages, dendrimers and clusters, are also possible and within the scope of the invention. “Nanoscaffold” refers generally to a nanostructure to which other molecules can be attached. RNA nanoparticles of various structural arrangements are described in, for example, WO 2005/003,293; WO 2007/016,507; WO 2008/039,254; WO 2010/148,085; WO 2012/170,372; WO 2015/042,101; WO 2015/196,146; WO 2016/168,784; and WO 2017/197,009, the contents of each of which are incorporated herein by reference.

RNA nanoparticles may contain multiple units that are themselves RNA nanostructures. For example, RNA nanoparticles may contain two or more trimers, tetramers, pentamers, hexamers, heptamers, octamers, or other polymers. The individual units may have any structure, such as those described above.

RNA/DNA hybrid nanoparticles that allow delivery of functional RNA molecules, such as ribozymes, riboswitches, and siRNA, have also been reported. RNA/DNA hybrid nanoparticles may have structures similar to those described above for RNA nanoparticles. RNA/DNA hybrid nanoparticles in which only the arms of the nanoparticle have been reported. RNA/DNA nanoparticles of various structural arrangements are described in, for example, US 2016/0208245; WO 2008/039,254; WO 2010/148,085; WO 2013/075,132; WO 2013/075,140; WO 2014/039,809; WO 2015/042,101; WO 2015/171,827; and WO/2017/139,758, the contents of each of which are incorporated herein by reference.

DNA nanoparticles capable of carrying cargo have also been described. DNA nanoparticles may be made from dendrimers. One common strategy for making DNA nanostructures is to build structures from dendrimers that contain double-stranded central regions and unpaired, single-stranded ends. Thus, one dendrimer can interact with up to four other molecules at its unpaired ends. Serial assembly of DNA dendrimers into polymeric structures allows formation of nanoparticles having various 3-dimensional arrangements. DNA nanoparticles of various structural arrangements are described in, for example, WO 2010/017,544; WO 2010/017,544; WO 2014/153,394; WO 2017/143,156; and WO 2017/143,171, the contents of each of which are incorporated herein by reference.

Nucleic acids are important macromolecules because the sequence of bases in a nucleic acid can carry information or impart functionality to the molecule. The nucleic acid nanoparticles of the invention may contain nucleic acid molecules that have functional information contained within their sequences. Additionally or alternatively, the sequences of nucleic acid molecules in the nanoparticles may play purely structural roles in coordinating the assembly and arrangement of molecules within the nanostructure.

Nucleic acid nanoparticles may contain naturally-occurring nucleotides, or they may contain chemically-modified nucleotides. Chemically-modified nucleotides are known in the art and described in, for example, WO 2018/118587, the contents of which are incorporated herein by reference. For example and without limitation, nucleic acid nanoparticles, therapeutics and aptamers may contain one or more of a 2′ fluoro, 2′ O-methyl, 2-thiouridine, 2′-O-methoxyethyl, 2′-amine, 5-methoxyuridine, pseudouridine, 5-methylcytidine, N1-methyl-pseudouridine, locked nucleic acid (LNA), morpholino, and phosphorothioate modification. Other modified nucleotides include 5caC, 5fC, 5hoC, 5hmC, 5meC/5fu, 5meC/5moU, 5meC/^{th}G, 5moC, 5meC/5camU, 5meC, _{Ψ}, 5meC/_{Ψ}, 5moC/5moU, 5moC/5meU, 5hmC/5meU, me1_{Ψ}, 5meC/me1_{Ψ}, 5moU, 5camU, m6A, 5hmC/_{Ψ}, 5moC/_{Ψ}, me6DAP, me4C, 5fu, 5-methoxyuridine, 2-aminoadenine, 2-thiocytosine, 2- thiothymine, 2-thiouracil, 3-methyladenine, 4-thiouracil, 5,6-dehydrouracil, 5-allylcytosine, 5- allyluracil, 5-aminoallylcytosine, 5-aminoallyluracil, 5-bromouracil, 5-ethynylcytosine, 5- ethynyluracil, 5-fluorouracil, 5-hydroxymethylcytosine, 5-hydroxymethyluracil, 5-iodouracil, 5- methylcytosine, 5-methyluracil, 5-propynylcytosine, 5-propynylcytosine, 5-propynyluracil, 5- propynyluracil, 6-O-methylguanine, 6-thioguanine, 7-deaza-8-azaguanine, 7-deazaadenine, 7- deazaguanine, 7-deazaguanine, 8-oxoadenine, 8-oxoguanine, 5-methylcytidine, pseudouridine, inosine, 2′-0-methyladenosine, 2′-Omethylcytidine, 2′-O-methylguanosine, 2′-O-methyluridine, 2′-0-methyl -pseudouridine, 2′-0-methyl 3′-phosphorothioate adenosine, 2′-0-methyl 3′- phosphorothioate cytidine, 2′-0-methyl 3′-phosphorothioate guanosine, 2′-0-methyl 3′- phosphorothioate uridine, a conformationally-restricted nucleotide, and 2′-0-methyl 3′- phosphorothioate pseudouridine. Representative modified nucleotides are shown below:

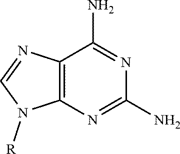

diaminopurine (DAP),

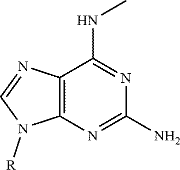

N⁶-methyl-2-aminoadenosine (me⁶DAP),

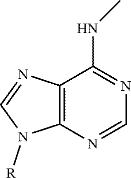

N⁶-methyladenosine (me⁶A),

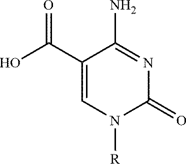

5-carboxycytidine (5caC),

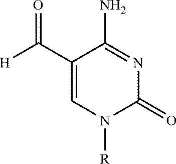

5-formylcytidine (5fC),

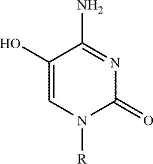

5-hydroxycytidine (5hoC),

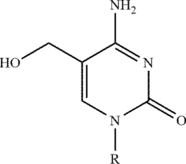

5-hydroxymethylcytidine (5hmC),

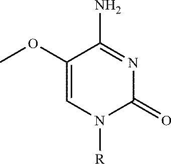

5-methoxycytidine (5moC),

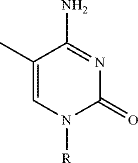

5-methylcytidine (5meC),

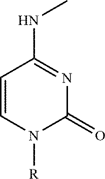

N⁴-methylcytidine (me⁴C),

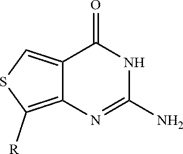

thienoguanosine (^{th}G),

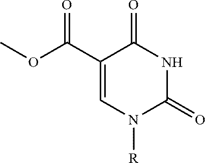

5-carboxymethylesteruridine (5camU),

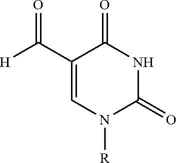

5-formyluridine (5fU),

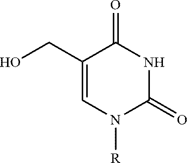

5-hydroxymethyluridine (5hmU),

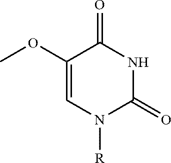

5-methoxyuridine (5moU),

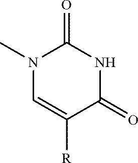

N¹-methylpseudouridine (me¹Ψ),

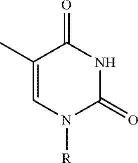

5-methyluridine (5meU), and

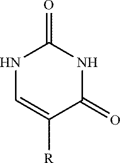

pseudouridine (T).

Modified nucleotides may have linkages other than phosphodiester bonds. For example, modified nucleotides may be linked by peptide, phosphorothioate, or phosphate bonds. Additionally, aptamers may contain one or more other chemical modifications described in WO 2018/118587.

Components of nucleic acid nanoparticles may be folded using DNA or RNA origami techniques to enhance stability or cell entry. DNA and RNA origami techniques are known in the art and described in, for example, U.S. Pat. No. 7,842,793; WO 2016/144755; Han et al., Single-stranded DNA and RNA origami, Science 15 Dec. 2017: Vol. 358, Issue 6369, eaao2648, DOI: 10.1126/science.aao2648, the contents of each of which are incorporated herein by reference. RNA origami is a method through which single-stranded RNA can be systematically folded into complex and molecularly defined two- and three-dimensional nanostructures using oligonucleotide hybridization and inter-strand cross-overs to dictate the final shape.

The use of RNA origami as a mRNA packaging system is within the scope of the invention, which contemplates exploration of its application as a therapeutic reagent both in vivo and in vitro for diagnostic, treatment, and/or research purposes for cancer and other genetically-related conditions.

Nucleic acid nanoparticles may contain additional modifications that minimize proteins or other molecules from adhering non-specifically to the nanoparticles. For example, nucleic acid nanoparticles and/or nucleic acid monomers contained therein may contain polyethylene glycol (PEG) moieties.

The nucleic acids of the nanoparticles may contain sugar modifications. For example and without limitation, the nucleic acids of the nanoparticles may contain one or more of 2′MOE, 2′OMe, 2′F, 2-′O-acetalesters, GMEBuOM, AMPrOM, AMEBuOM, PivOM, 2′ amino locked nucleic acids (LNA) modified with amines or peptides mentioned above, 2′-O-[N,N-dimethylamino)ethoxy]ethyl, 2′-N-[N,N-dimethylamino)ethoxy]ethyl, 2′-N-imidazolacetyamide, 2′-O-[3-(guanidinium)propyl], 2′-N-[3-(guanidinium)propyl], 2′-O-[3-(guanidinium)ethyl], 2′-N-[3-(guanidinium)ethyl], 2′-O-(N-(aminoethyl)carbamoyl)methyl, 2′-N-(N-(aminoethyl)carbamoyl)methyl, 2′-O-[N-(2-((2-aminoethyl)amino)ethyl)]acetamide, 2′-N-[N-(2-((2-aminoethyl)amino)ethyl)]acetamide, 2′-N-2,2,3,3,4,4,5,5,6,6,7,7,8,8,8-pentadecafluorooctanamide, 2′-N-imidazolacetamide, 2′-O-imidazole methyl, 2′-N-guanidylbenzylamide, and 4′-C-guanidinincarbohydrazidomethyl, 2′-O-imidazolemethyl, 2′-N-imidazolemethylamine ethyl.

The nucleic acids of the nanoparticles may contain modified phosphorus-containing backbones. For example and without limitation, the nucleic acids of the nanoparticle may contain one or more of a 3-(2-nitrophenyl)-propyl phosphoramidite linkage, a 3-phenylpropyl phosphoramidite linkage, an alkyl phosphorothioate linkage, an aminobutyl phosphoramidite linkage, an aryl phosphorothioate linkage, a dimethylamino phopsphoramidite linkage, a guanidinobutylphosphoramidate linkage, and a phosphorothioate linkage.

Specific modifications to nucleic acid nanoparticles that promote the functionality of the attached cargo molecules are described in more detail as relevant in the following sections of the specification.

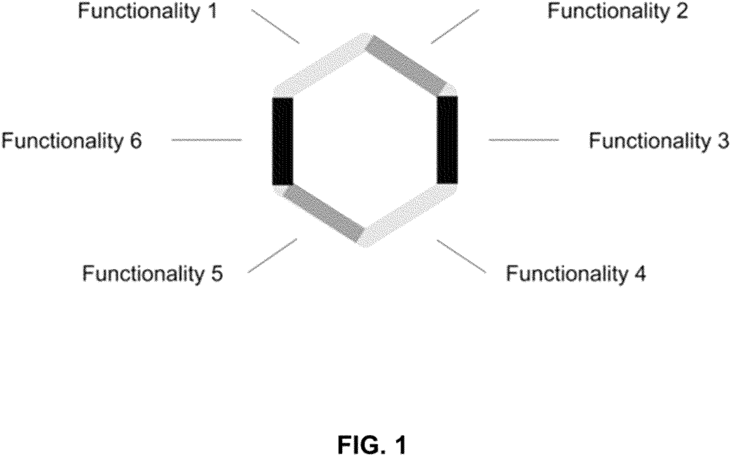

FIG. 1 is a schematic of a nucleic acid nanoparticle according to an embodiment of the invention. As shown, the nanoparticle is a hexamer of six nucleic acids, each having a different functionality.

### Cargo Molecules

Compositions of the invention may contain one or more cargo molecules attached to a nucleic acid nanoparticle. The cargo molecule may be any type of molecule that provides a therapeutic benefit. For example and without limitation, the cargo molecule may be or include a nucleic acid, such as DNA, RNA, or a DNA/RNA hybrid, peptide, polypeptide, protein, antibody, label, reporter, stabilizing agent, targeting moiety, or other therapeutic agent. The RNA molecule may be a mRNA molecule, a lnRNA molecule, a miRNA molecule, a siRNA molecule, or a shRNA molecule. Nucleic acid cargo molecules may contain modified nucleotides and/or may have modified phosphorus-containing linkages, such as any of the nucleotides and linkages described above in relation to nucleic acid nanoparticles. Therapeutic agents may be small molecule agents, e.g., organic molecules having a mass of less than 1 kDa, 1.5 kDa, or 2 kDa, or biologic agents, e.g., agents that include proteins, peptides, antibodies, nucleic acids, and combinations thereof. The cargo molecule may be a chemotherapeutic. For example and without limitation, chemotherapeutics include actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, or vinorelbine. Additional chemotherapeutic agents are described in U.S. Publication 2017/0121708, the contents of which are incorporated herein by reference.

For diagnostic procedures, it is useful to identify target cells, such as cancer cells or infected cells. Thus, the cargo molecule may be a detectable label that facilitates identification of a target cell. For example and without limitation, the label may be a fluorescent molecule, optical label, light-emitting molecule, phosphorescent molecule, dye, radiolabel, enzyme, or substrate.

In general, different agents are effective at different concentrations. Common measures for the concentration at which a therapeutic agent is effective are the IC₅₀ and EC₅₀, although other measures may be used. Consequently, when delivering multiple therapeutic agents with a single composition, it is advantageous to be able to provide the therapeutic agents in different amounts so that each agent is supplied in an amount at or near the concentration that optimizes its effectiveness. Thus, different cargo molecules may be attached to nanoparticles in different amounts to facilitate delivery of each cargo molecules at an optimal concentration.

The different cargo molecules may act on the same cell. Alternatively or additionally, the different cargo molecules may act on different cells. The different cells may be in proximity to each other. For example, the different cells may be in contact with each other, may be in the same tissue, or may otherwise be associated with each other.

The different cargo molecules may alter the activities of different pathways. The pathways may be altered in the same cell or in different cells. The different pathways may be involved in a common disease or disorder. For example, aberrant activity in each pathway may be associated with the disease or disorder, such as cancer. The different cargo molecules may act on cells and/or may alter activities of different pathways at different amounts.

The nanoparticle may have different cargo molecules bound at different stoichiometries. For example, the two cargo molecules may be linked to the nanoparticle in stoichiometric ratio other than 1:1, such as 1:2, 1:3, 1:4, 1:5, 2:3, 3:4, etc.

The skilled artisan will realize that while exemplified for cancer, this approach of targeting multiple disease pathways using different amounts of different drugs applies outside of cancer. For example, any disease in which multiple pathways in a single cell need to be targeted with different drugs at different concentrations is within the scope of the invention. Other exemplary diseases include tuberculosis, leprosy, malaria, HIV/AIDS, autoimmune diseases, inflammatory diseases, e.g., Crohn’s disease and inflammatory bowel disease, infections, infectious diseases, hereditary angioedema (HAE), multiple sclerosis, spinal cord injury, dyslipidemia, hypertension, neurological diseases, e.g., Alzheimer’s disease and Parkinson’s disease, ulcers, psoriasis, and hepatitis.

The cargo molecule may be a CRISPR component. The CRISPR system is a prokaryotic immune system that provides acquired immunity against foreign genetic elements, such as plasmids and phages. CRISPR systems include one or more CRISPR-associated (Cas) proteins that cleave DNA at clustered, regularly-interspersed palindromic repeat (CRISPR) sequences. Cas proteins include helicase and exonuclease activities, and these activities may be on the same polypeptide or on separate polypeptides. Cas proteins are directed to CRISPR sequences by RNA molecules. A CRISPR RNA (crRNA) binds to a complementary sequence in the target DNA to be cleaved. A transactivating crRNA (tracrRNA) binds to both the Cas protein and the crRNA to draw the Cas protein to the target DNA sequence. Not all CRISPR systems require tracrRNA. In nature crRNA and tracrRNA occur on separate RNA molecules, but they also function when contained a single RNA molecule, called a single guide RNA or guide RNA (gRNA). The one or more RNAs and one or more polypeptides assemble inside the cell to form a ribonucleoprotein (RNP). CRISPR systems are described, for example, in van der Oost, et al., CRISPR-based adaptive and heritable immunity in prokaryotes, Trends in Biochemical Sciences, 34(8):401-407 (2014); Garrett, et al., Archaeal CRISPR-based immune systems: exchangeable functional modules, Trends in Microbiol. 19(11):549-556 (2011); Makarova, et al., Evolution and classification of the CRISPR-Cas systems, Nat. Rev. Microbiol. 9:467-477 (2011); and Sorek, et al., CRISPR-Mediated Adaptive Immune Systems in Bacteria and Archaea, Ann. Rev. Biochem. 82:237-266 (2013), the contents of each of which are incorporated herein by reference.

CRISPR-Cas systems have been placed in two classes. Class 1 systems use multiple Cas proteins to degrade nucleic acids, while class 2 systems use a single large Cas protein. Class 1 Cas proteins include Cas1O, Cas10d, Cas3, Cas5, Cas8a, Cmr5, Cse1, Cse2, Csf1, Csm2, Csx11, Csy1, Csy2, and Csy3. Class 2 Cas proteins include C2c1, C2c2, C2c3, Cas4, Cas9, Cpf1/Cas12a, and Csn2.

CRISPR-Cas systems are powerful tools because they allow gene editing of specific nucleic acid sequences using a common protein enzyme. By designing a guide RNA complementary to a target sequence, a Cas protein can be direct to cleave that target sequence. In addition, although naturally-occurring Cas proteins have endonuclease activity, Cas proteins have been engineered to perform other functions. For example, endonuclease-deactivated mutants of Cas9 (dCas9) have been created, and such mutants can be directed to bind to target DNA sequences without cleaving them. dCas9 proteins can then be further engineered to bind transcriptional activators or inhibitors. As a result, guide sequences can be used to recruit such CRISPR complexes to specific genes to turn on or off transcription. Thus, these systems are called CRISPR activators (CRISPRa) or CRISPR inhibitors (CRISPRi). CRISPR systems can also be used to introduce sequence-specific epigenetic modifications of DNA, such acetylation or methylation. The use of modified CRISPR systems for purposes other than cleavage of target DNA are described, for example, in Dominguez, et al., Beyond editing: repurposing CRISPR-Cas9 for precision genome regulation and interrogation, Nat. Rev. Cell Biol. 17(1):5-15 (2016), which is incorporated herein by reference.

The compositions of the invention may include any component of a CRISPR system, such as those described above. For example and without limitation, the CRISPR component may be one or more of a helicase, endonuclease, transcriptional activator, transcriptional inhibitor, DNA modifier, gRNA, crRNA, or tracrRNA. The CRISPR component may contain a nucleic acid, such as RNA or DNA, a polypeptide, or a combination, such as a RNP. The CRISPR nucleic acid may encode a functional CRISPR component. For example, the nucleic acid may be a DNA or mRNA. The mRNA may be complexed with antisense DNA to stabilized the mRNA. The CRISPR nucleic acid may itself be a functional component, such as a gRNA, crRNA, or tracrRNA. Nucleic acid-based CRISPR components may contain modified nucleotides, such as those described elsewhere in this application. Nucleic acid-based CRISPR components may be folded using DNA or RNA origami techniques to enhance stability or cell entry.

The CRISPR component may alter expression of a target in a cell. For example, the CRISPR component may increase or decrease expression of a target in a cell.

The composition may include an element that induces expression of the CRISPR component. For example, expression of the CRISPR component may be induced by an antibiotic, such as tetracycline, or other chemical. Inducible CRISPR systems have been described, for example, in Rose, et al., Rapidly inducible Cas9 and DSB-ddPCR to probe editing kinetics, Nat. Methods, 14, pages 891-896 (2017); andCao, et al., An easy and efficient inducible CRISPR/Cas9 platform with improved specificity for multiple gene targeting, Nucleic Acids Res. 14(19):e149 (2016). The inducible element may be part of the CRISPR component 105, or it may be a separate component attached to nucleic acid nanoparticle 103.

The cargo molecule may be a chimeric antigen receptor (CAR). CARs are engineered receptors that can confer a desired specificity onto an immune cell, such as a T cell. Typically, CARs are designed to bind to molecular markers on cancer cells, such as a tumor associated antigen (TAA). CAR-expressing T cells (CAR-T cells) are useful in treating cancer because they allow rapid generation of a population of T cells that target and kill specific tumor cells. CAR-T cells can also be programmed to destroy cells infected with a pathogen, such as a virus or bacterium. CAR-T cells can be made by introducing a CAR either into a patient’s own T cells (autologous CAR-T cells) or into a T cells from another donor (allogeneic CAR-T cells). CARs may also be expressed in other cells of the immune system, such as NK cells, NKT cells or macrophages. The use of CARs in cancer immunotherapy is described in, for example, Smith et al., Chimeric antigen receptor (CAR) T cell therapy for malignant cancers: Summary and perspective, J. Cellular Immnother. 2:59-68 (2016), doi.org/10.1016/j.jocit.2016.08.001; and Ye et al., Engineering chimeric antigen receptor-T cells for cancer treatment, Mol. Cancer, 17:32 (2018), doi.org/10.1186/s12943-018-0814-0, the contents of each of which are incorporated herein by reference.

Structurally, CARs include an extracellular domain that binds to an antigen, a transmembrane domain, and an intracellular signaling domain. The extracellular domain of a CAR is typically derived from a single-chain variable fragment (scFv) of an immunoglobulin. The transmembrane domain links the scFv extracellular antigen-binding domain to the intracellular signaling domain and may be derived from CD28. The intracellular domain includes a CD3-zeta chain and may contain additional co-stimulatory domains derived from other signaling molecules. For example and without limitation, the intracellular domain may include portions from one or more of 4-1BB, CD27, CD28, CD134, CD137, CD70, CD80, CD86, and OX40. The intracellular domain may also interact with nuclear factor of activated T cell (NFAT) to induce expression of a cassette of IL-12 genes in a mechanism called T cell redirected for universal cytokine killing (TRUCK).

The chimeric antigen receptor may recognize a molecular marker associated with cancer or a tumor. The marker may be a protein or other molecule that is expressed only in tumor cells or that is expressed to higher levels in tumor cells. For example and without limitation, the chimeric antigen receptor may recognize 5T4, alpha V beta 6 integrin, AXL, BCMA, C4.4A, CA6, CA9, Cadherin 6, CAIX, carcinoembryonic antigen (CEA), CD123, CD138, CD16A, CD171, CD171, CD19, CD20, CD22, CD28, CD3, CD30, CD326 (EPCAM) CD32B, CD33, CD38, CD64, CD79B, CEA, c-Kit, c-MET, criptoprotein, CS1, DLL3, EDNRB, EFNA4, EGFR, EGFR, EGFRvIII, ENPP3, epithelial cell adhesion molecule (EpCAM), ErbB, ErbB2, erythropoietin-producing hepatocellular A2 receptor (EphA2), FAP, FGFR2, FGFR3, fibroblast activation protein (FAP), FLT3, folate receptor-alpha, GD2, glycosylated MUC-1, glypican 3, gp100, gpA33, GPC3, GPNMB, GUCY2C, HER2, HER3, IGF-1R, IL-6R, interleukin 13 receptor α (IL13Rα), kappa light chain, L1 cell adhesion molecule (L1CAM), Lewis Y, LIV-1, LRRC15, MAGE family members, mesothelin, MET, MSLN, MUC1, MUC16, NaPi2b, Nectin-4, NKG2D, NOTCH3, NY-ESO-1, p-CAD, PDGFR, prostate specific cancer antigen (PSCA), prostate-specific membrane antigen (PSMA), PTK7, RORI, SLC44A4, SLITRK6, STEAP1, TF, TIM-1, TROP-2, vascular endothelial growth factor receptor 2 (VEGF-R2), or VEGF. The chimeric antigen receptor may recognize a neoantigen, i.e., a protein or other molecule that is expressed in a mutant form in tumor cells.

### Linkers

In compositions of the invention, cargo molecules may be attached to nucleic acid nanoparticles, functional elements, or both via linkers. The attachments may be covalent or noncovalent. The attachments may be reversible. Particularly useful are reversible attachments that bind the cargo molecule to the nanoparticle or functional element while the composition is being transported to a target and then release the cargo molecule from the nanoparticle or functional element when the cargo molecule has been delivered to the target.

Examples of reversible linkers that may be used in compositions of the invention include acetals, acid-labile linkages, amino esters, azide-alkyne bonds, biotin-streptavidin linkages, disulfide bonds, dithiopyridyls, enzymatically-cleavable linkages, hydrazones, imines, maleic anhydrides, maleimides, nucleotide base pairs, ribozyme linkages, Schiff-base linked imidizoles, thioethers, and triethylene glycol. Such are linkers are known in the art and described in, for example, Paredes E. et al, RNA labeling, conjugation and ligation, Methods 54:2, June 2011, Pages 251-259, doi.org/10.1016/j.ymeth.2011.02.008; Pramod, P. S. et al., Real-Time Drug Release Analysis of Enzyme and pH Responsive Polysaccharide Nanovesicles, J Phys Chem (2015) B 119, 10511-10523, doi:10.1021/acs.jpcb.5b05795; Oishi, M., et al., Lactosylated poly(ethylene glycol)-siRNA conjugate through acid-labile beta-thiopropionate linkage to construct pH-sensitive polyion complex micelles achieving enhanced gene silencing in hepatoma cells, J Am Chem Soc (2005) 127, 1624-1625, doi:10.1021/ja044941d; Maier, K. and Wagner, E. Acid-labile traceless click linker for protein transduction, J Am Chem Soc (2012) 134, 10169-10173, doi:10.1021/ja302705v; Hoeprich, S. et al., Bacterial virus phi29 pRNA as a hammerhead ribozyme escort to destroy hepatitis B virus, Gene Ther (2003) 10, 1258-1267, doi:10.1038/sj.gt.3302002; and Chan, K. et al. Exploiting the Protein Corona from Cell Lysate on DNA Functionalized Gold Nanoparticles for Enhanced mRNA Translation, ACS Appl. Mater. Interfaces 2017, 9, 10408-10417, DOI: 10.1021/acsami.6b15269, the contents of each of which are incorporated herein by reference.

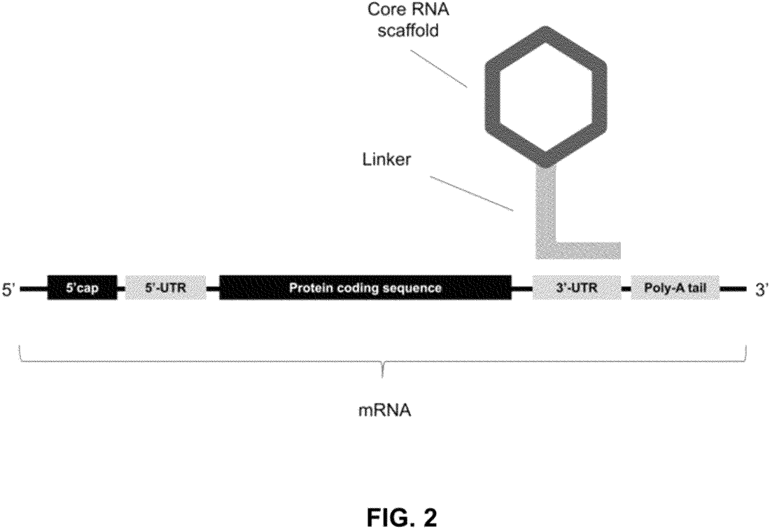

FIG. 2 is a schematic of a composition that includes a nucleic acid nanoparticle, linker, and mRNA molecule according to an embodiment of the invention. The mRNA molecule includes a 5′ cap, 5′ UTR, coding sequence, 3′ UTR, and polyA tail. The linker binds to the 3′ UTR of the mRNA molecule, but could also bind to other regions, including the 5′ cap, 5′ UTR, coding sequence, and polyA tail.

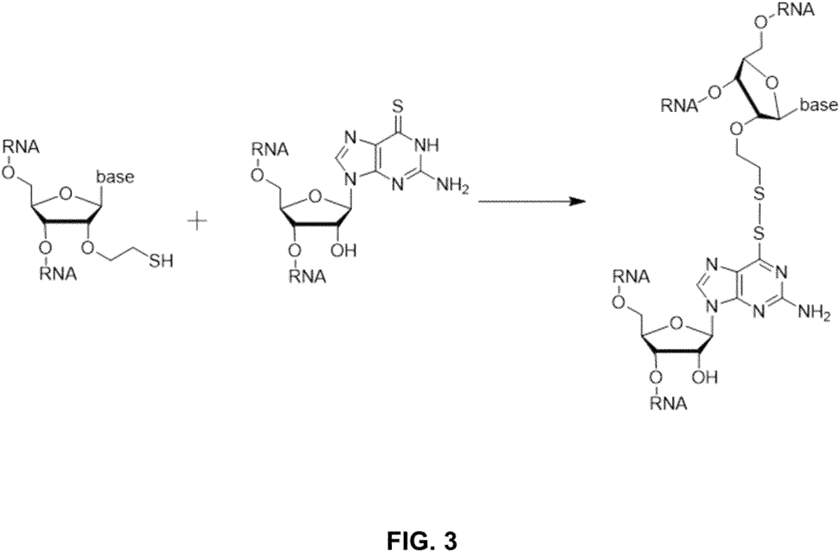

FIG. 3 shows formation of a disulfide linkage that can be used to attach a cargo molecule to a nucleic acid nanoparticle according to an embodiment of the invention. One molecule is an oligonucleotide bearing a 2′ thiol moiety, and the other is an oligonucleotide having a 6-thioguanosine. In the reaction shown, either oligonucleotide may be the cargo molecule, and either may be the nucleic acid nanoparticle.

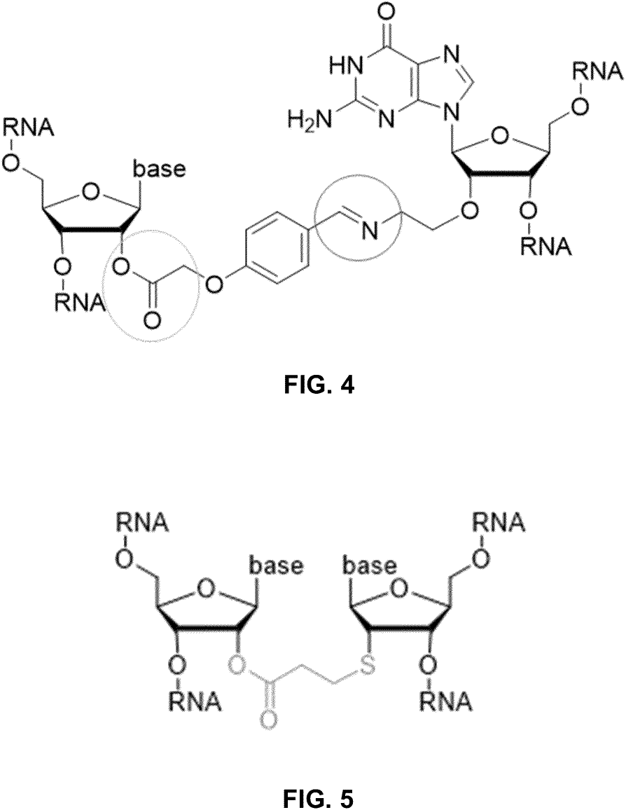

FIG. 4 shows an example of a linker that can be used to attach a cargo molecule to a nucleic acid nanoparticle according to an embodiment of the invention. The region circled in orange is a chemical bond that is enzymatically cleavable by esterases. The region circle in blue is pH-sensitive chemical bond that is cleavable by acid.

FIG. 5 shows an example of a linker that can be used to attach a cargo molecule to a nucleic acid nanoparticle according to an embodiment of the invention. The region shown in yellow is a β-thiopropionate moiety that is cleavable by acid.

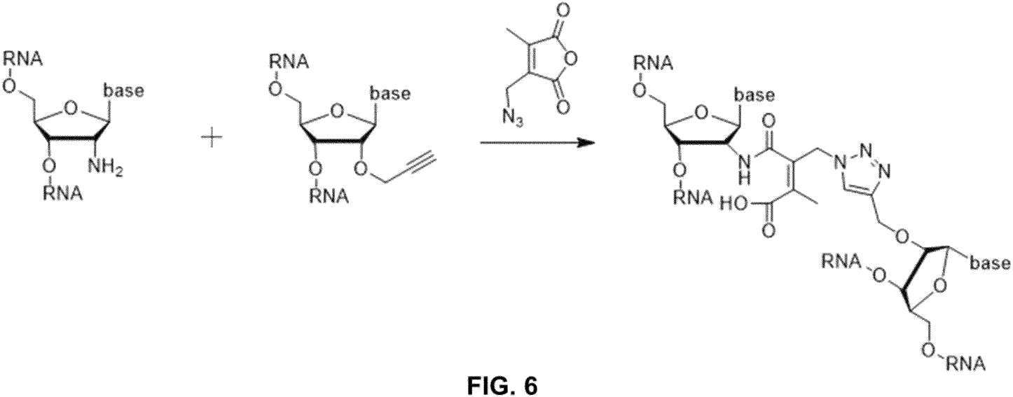

FIG. 6 shows formation of an azidomethyl-methylmaleic anhydride that can be used to attach a cargo molecule to a nucleic acid nanoparticle according to an embodiment of the invention. The azidomethyl-methylmaleic anhydride linkage is cleavable by acid.

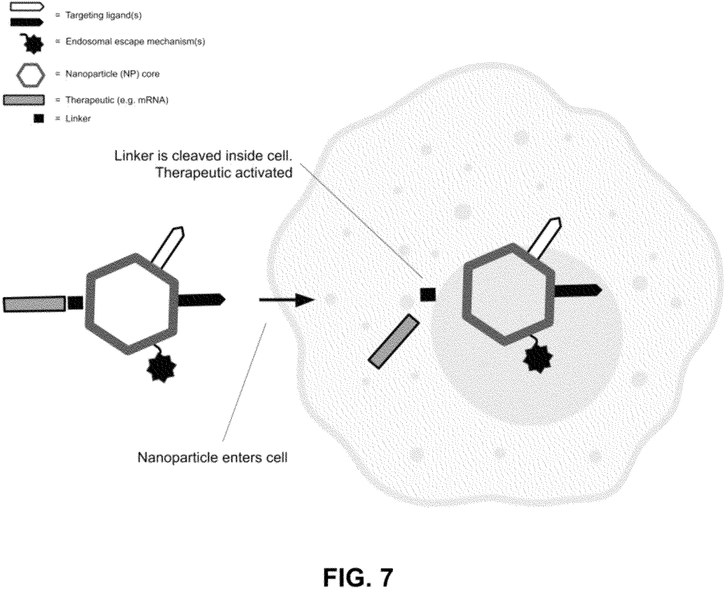

FIG. 7 is a schematic showing cleavage of a composition according to an embodiment of the invention. The composition includes targeting ligands (white pentagon and black pentagon), and endosomal escape mechanism (8-pointed star), nucleic acid nanoparticle core (hexagon), therapeutic cargo molecule (grey rectangle), and linker (black square). The composition circulates intact in the body and is directed to a target cell by the targeting ligands. When the composition is internalized by the target cell, the linker is cleaved, resulting in the release and activation of the therapeutic cargo molecule.

### Compacted RNA Molecules, Including mRNA Molecules

As described above, the cargo molecules in compositions of the invention may be RNA molecules. However, delivery of RNA molecules, including mRNAs, is challenging due to the presence of ribonucleases in the blood and other extracellular environments in the body. In addition, compared to DNA, RNA is more labile. Such problems are overcome in certain compositions of the invention that contain RNA molecules that assume a compacted form.

The compacted RNA molecule may be a mRNA molecule, a lnRNA molecule, a miRNA molecule, a siRNA molecule, or a shRNA molecule.

A compacted form may include any form that changes the three-dimensional structure of the RNA molecule. For example and without limitation, compacted RNA molecules may include one or more of the following: folds; secondary structures; interactions with other molecules, such as RNA, DNA, peptides, polypeptides, or proteins; and altered phosphorus-containing backbones.

RNA origami refers to the nanoscale folding of RNA that allows RNA molecules to assemble into particular shapes. RNA origami, RNA folding, and mechanisms for producing compacted RNA molecules are described in, for example, C. Geary, P. W. K. Rothemund, E. S. Andersen. A single-stranded architecture for cotranscriptional folding of RNA nanostructures. Science, 2014; 345 (6198): 799 DOI: 10.1126/science.1253920; Dongran, H., et al., Single-stranded DNA and RNA origami, Science 15 Dec. 2017: Vol. 358, Issue 6369, eaao2648, DOI: 10.1126/science.aao2648; Sparvath SL, et al., Computer-Aided Design of RNA Origami Structures, Methods Mol Biol. 2017;1500:51-80, DOI: 10.1007/978-1-4939-6454-3_5; Krissanaprasit A, et al., Genetically Encoded, Functional Single-Strand RNA Origami: Anticoagulant, Adv Mater. 2019 May;31(21):e1808262. doi: 10.1002/adma.201808262; Geary, C. et al, Promoting RNA helical stacking via A-minor junctions, Nucleic Acids Res. 2011 Feb; 39(3): 1066-1080, doi: 10.1093/nar/gkq748; Klein, DJ, et al, The kink-turn: a new RNA secondary structure motif, EMBO J. 2001 Aug 1; 20(15): 4214-4221, doi: 10.1093/emboj/20.15.4214; Wang P. et al., RNA-DNA hybrid origami: folding of a long RNA single strand into complex nanostructures using short DNA helper strands, Chem Commun (Camb). 2013 Jun 18;49(48):5462-4. doi: 10.1039/c3cc41707g; Endo M., et al, Preparation of chemically modified RNA origami nanostructures, Chemistry. 2014 Nov 17;20(47):15330-3. doi: 10.1002/chem.201404084; Okada H, et al, Novel complementary peptides to target molecules, Anticancer Res. 2011 Jul;31(7):2511-6; Kim H, Self-assembled Messenger RNA Nanoparticles (mRNA-NPs) for Efficient Gene Expression, Sci Rep. 2015; 5: 12737, doi: 10.1038/srep12737; and Sparvath S.L., Geary C.W., Andersen E.S. (2017) Computer-Aided Design of RNA Origami Structures. In: Ke Y., Wang P. (eds) 3D DNA Nanostructure. Methods in Molecular Biology, vol 1500. Humana Press, New York, NY; and International Patent Publication Nos. WO 2005/003,293; WO 2007/016,507; WO 2008/039,254; WO 2010/148,085; WO 2012/170,372; WO 2015/042,101; WO 2015/196,146; WO 2016/168,784; and WO 2017/197,009, the contents of each of which are incorporated herein by reference.

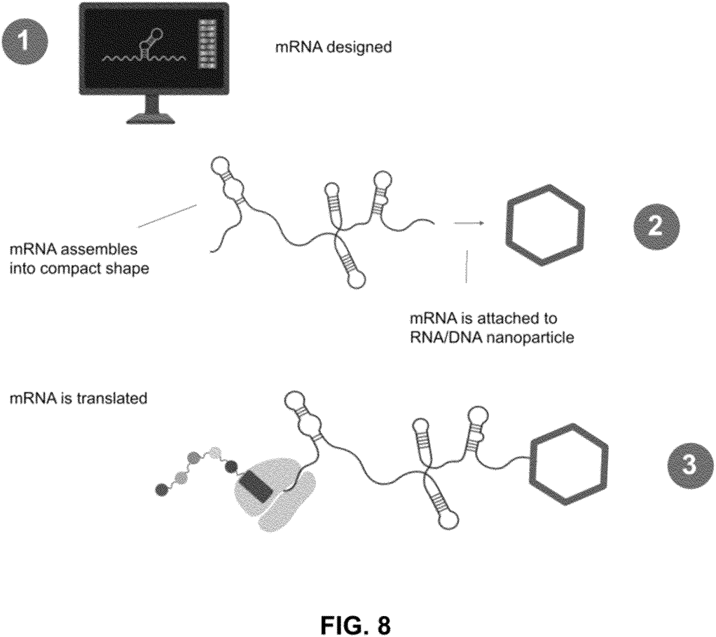

FIG. 8 shows a method of using RNA origami to produce compacted mRNA molecules. The mRNA origami is computationally designed, encoded as a synthetic DNA gene, and 3) synthesized using RNA-polymerase. RNA origami can be designed as a single strand (ssRNA) or double strand of RNA (dsRNA), and produced via transcription or oligonucleotide synthesis.

Various strategies can be used to produce RNA molecules suitable for RNA origami or generally to produce compact RNA structures. RNA molecules may contain intrinsic structures, such as one or more of the following: codon substitution, including changes to sequence that improve folding but do not affect the sequence of the encoded polypeptide; complementary base pairing; chemical modifications that alter the charge under physiological conditions; and RNA structural motifs, such as coaxial stacks, dovetail seams, hairpin loops, helixes, kink turns, kissing loops, pseudoknots, tetra loops, and wobble pairs.

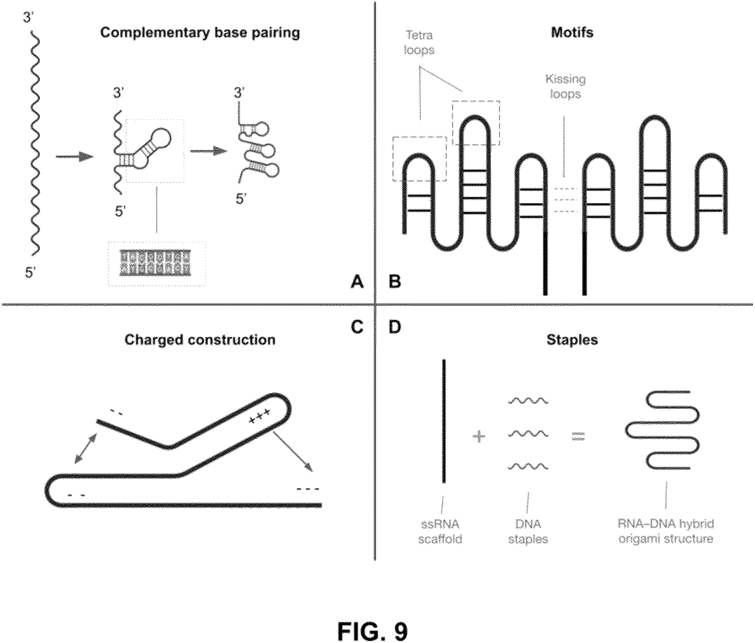

FIG. 9 shows examples of mRNA origami folding principles. Strategies to promote mRNA folding include complementary base pairing (upper left panel); motifs, such as tetra loops and kissing loops (upper right panel); charged interactions (lower left panel); and use of additional molecules that promote folding (lower right panel).

Another method of producing compacted RNA molecules is to apply pressure prior to, during, or following synthesis of RNA to increase the proximity of bases, giving maximum chance for bonding between bases or regions of RNA.

Additional molecules may be combined with RNA molecules to promote compaction of the RNA molecules. Thus, other molecules may serve as packing components. The other molecule or molecules may act as nodes that form loops to make more compact RNA structures. For example and without limitation, the other molecule may be DNA, RNA, a peptide, a polypeptide, a targeting molecule, a protein, or a riboswitch. When a nucleic acid serves as a packing component, it may be single-stranded or double-stranded, or it may contain both single-stranded and double-stranded regions. Such nucleic acids may be described as staples or pins. The packing component may be a histone or modified histone that interacts with RNA to create RNA nucleosomes or RNA chromatin. The tail domains of core histone proteins can be modified, for example, by altering the basic amino acid residues and/or including a free peptide similar to the N-terminal tail domain, to increase affinity for RNA rather than DNA.

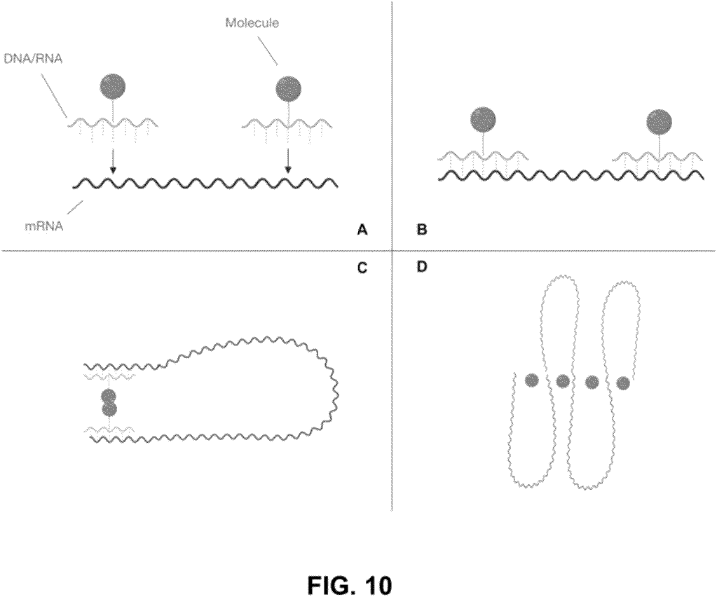

FIG. 10 shows the use of “pins” to hold mRNA in a compacted configuration. The pin may consist of a nucleic acid, such as RNA or DNA, attached to another molecule that binds that can dimerize. The nucleic acid hybridizes to mRNA via base pairing (upper left and upper right panels). The other molecule then dimerizes, causing the mRNA to assume a looped conformation (lower left panel). The presence of multiple dimerized “pins” causes the RNA molecule to assume a flattened, serpentine structure (lower right panel).

The packing component may include two or more molecules that interact with each other, such as two molecules that show high affinity or two peptides that bind each other. Interactions between the two molecules may be reversible and/or regulatable. When the two molecules bind to each other, the RNA may assume a compacted form, and when the two molecules are not bound, the mRNA may assume a more open or flexible configuration. In certain embodiments, the configuration of the RNA molecule can be regulated to transition between a compacted state and an open state.

In certain embodiments of the invention, RNA molecules self-assemble into nanoparticles. The self-assembled RNA nanoparticles may contain functional moieties. For example and without limitation, self-assembled RNA nanoparticles may contain elements that promote endosomal escape, elements that alter binding to plasma proteins, elements that promote cellular internalization, such as those described elsewhere in this application.

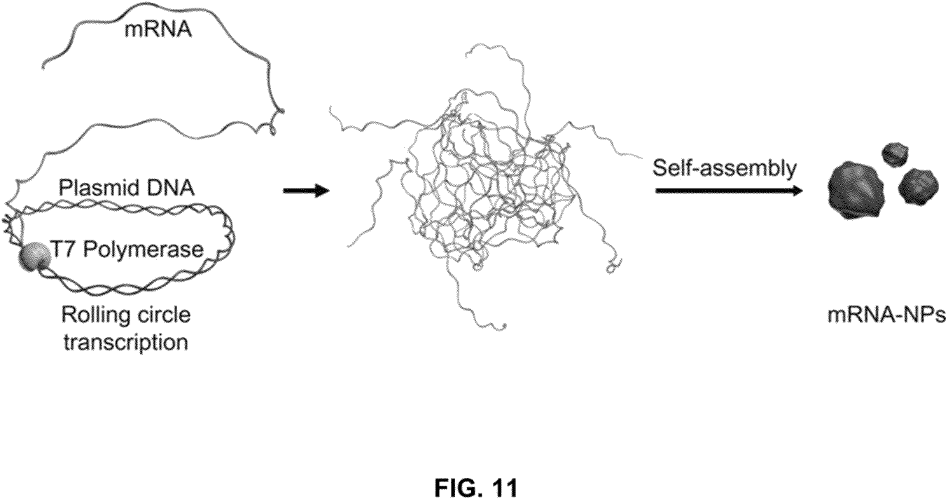

FIG. 11 shows mRNA molecules that self-assemble into nanoparticles. mRNA molecules are transcribed from plasmid DNA, and the free mRNA molecules fold through a series of binding interactions, such as base pairing, into compact nanoparticles.

mRNA molecules may contain modifications of the 5′ cap, 3′ polyadenylated tail, and/or nucleotides that promote a compacted configuration. In addition to affecting mRNA structure, some of the aforementioned modifications have other advantageous properties. For example, modification of the 5′ cap and/or 3′ polyadenylated tail, including elongation of the polyadenylated tail, can increase mRNA stability. Incorporation of modified nucleotides, such as pseudouridine and 5-methylcytidine, can reduce immunogenicity of mRNA molecules. See, e.g., Kariko K, et al., Incorporation of pseudouridine into mRNA yields superior nonimmunogenic vector with increased translational capacity and biological stability, Mol Ther. 2008 Nov;16(11):1833-40. doi: 10.1038/mt.2008.200; Kariko K. et al., Generating the optimal mRNA for therapy: HPLC purification eliminates immune activation and improves translation of nucleoside-modified, protein-encoding mRNA, Nucleic Acids Res (2011) 39, e142, doi:10.1093/nar/gkr695, the contents of each of which are incorporated herein by reference.

The compacted RNA molecules may contain any of the modified nucleotides described above.

Another approach to achieve more compact mRNA molecules is to split a coding sequence into two or more separate mRNA molecules that encode sub-domains of a polypeptide that assemble into a functional protein post-translationally.

### Regulating Expression of mRNA Molecules

Another challenge in the use of mRNA-based therapeutics and diagnostics is ensuring that the mRNA is expressed in the right cells at the right time. For example, some cancer therapies include mRNAs that encode products that lead to self-destruction of tumor cells, and off-target expression of such mRNAs could be catastrophic. On the other hand, a therapeutic mRNA that does reach its intended target will be ineffectual if it is not expressed at sufficient levels. The invention solves such problems by providing compositions that include mRNA molecules attached to nucleic acid nanoparticles that regulate the expression of the attached mRNA molecules.

Nucleic acid nanoparticles in compositions of the invention may regulate the expression of cargo mRNA molecules in a variety of ways. As described in more detail below, the nucleic acid nanoparticle may do one or more of the following: promote mRNA translation, inhibit mRNA degradation, promote mRNA degradation, or promote degradation of the nanoparticle. Moreover, the regulation may depend on environmental context. Thus, in certain embodiments, the nucleic acid nanoparticle permits or promotes expression of cargo mRNA molecules in one cell type and inhibits expression of the mRNA molecules in another cell type.

The nucleic acid nanoparticle may regulate expression of cargo mRNA molecules by promoting translation. The nanoparticle may promote enhance translation under specific environmental conditions. For example and without limitation, the nanoparticle may contain one or more of the following components: a riboswitch or regulatory segment that binds a ligand to promote translation of the mRNA molecule in the presence of the ligand; an element, such as an internal ribosome entry site, that recruits translation machinery or translation initiation factors to the mRNA molecule; an aptamer that facilitates delivery of the mRNA molecule to the cytoplasm for translation by cytoplasmic ribosomes; and a ribozyme that splices the mRNA molecule or otherwise activates it.

Alternatively or additionally, the nucleic acid nanoparticle may promote mRNA expression indirectly by inhibiting degradation of the mRNA molecules. For example, the nucleic acid nanoparticle may have a nuclease inhibitor, such as a RNase inhibitor or DNase inhibitor, incorporated into the nanoparticle. For example and without limitation, the nuclease inhibitor may be a polycation, barstar, or ribonuclease inhibitor. Incorporation of RNase inhibitors into other materials is described in, for example, Santos-Cancel M and White RJ, Collagen Membranes with Ribonuclease Inhibitors for Long-Term Stability of Electrochemical Aptamer-Based Sensors Employing RNA, Anal Chem. 2017 May 16;89(10):5598-5604. doi: 10.1021/acs.analchem.7b00766, the contents of which are incorporated herein by reference. Another approach is to incorporate inhibitory RNAs, such as lnRNAs (long non-coding RNAs), miRNAs, shRNAs, or siRNAs, that target cellular mRNA molecules that encode nucleases, such as RNase. By including such inhibitory RNAs, the nanoparticles prevent translation of nucleases that would otherwise degraded cargo mRNA molecules.

The nucleic acid nanoparticles may also promote selective degradation of cargo mRNA molecules so that they are not expressed in certain cell types. For example and without limitation, the nucleic acid nanoparticle may contain or encode one or more of the following: lnRNA, miRNA, shRNA, siRNA, and an antisense oligonucleotide. The nucleic acid nanoparticle may also include a mRNA molecule that encodes a nuclease, such as RNase, that leads to degradation of the cargo mRNA molecule under appropriate conditions. Expression of the mRNA molecule encoding the nuclease may itself be regulated by another of the mechanisms described herein.

Expression of cargo mRNA molecules may also be regulated by including components that promote degradation of structural nucleic acids of the nanoparticle. Thus, the nanoparticle may contain one or more components that lead to self-destruction in one cell type but not another cell type. For example and without limitation, the structural nucleic acids of the nucleic acid nanoparticle may contain one or more of the following: a target sequence that attracts a nuclease, such as Dicer; a lnRNA; a miRNA; a shRNA, a siRNA;

The cargo mRNA molecule itself may contain a target site that leads to its degradation in one cell type but not another cell type. For example, cellular miRNAs display disease-specific expression patterns and may be expressed in healthy cells but not diseased cells. By incorporating miRNA target sites into mRNA molecule, the mRNA molecule is degraded in healthy cells but not in diseased cells. If the mRNA encodes a polypeptide that promotes apoptosis, the composition can be used to kill diseased cells but leave healthy cells unaffected. The use of mRNAs containing target sites for miRNA has been described in, for example, Jain R, et al., MicroRNAs Enable mRNA Therapeutics to Selectively Program Cancer Cells to Self-Destruct, Nucleic Acid Ther. 2018 Oct;28(5):285-296, doi: 10.1089/nat.2018.0734, the contents of which are incorporated herein by reference.

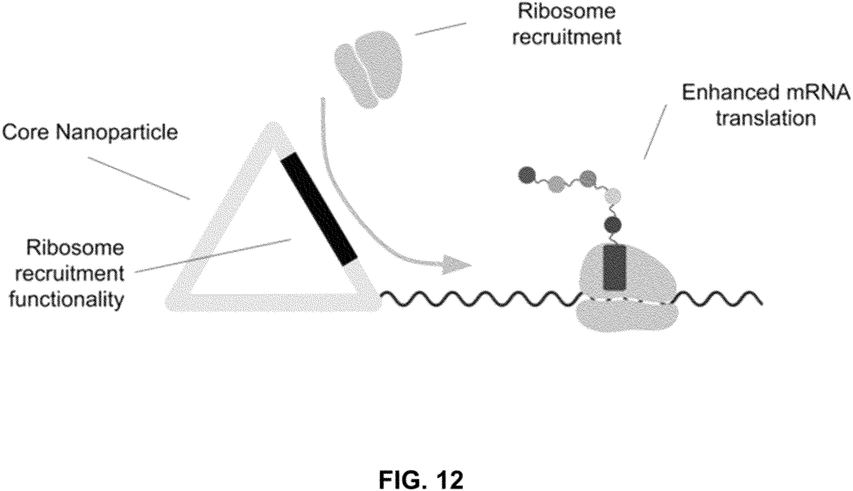

FIG. 12 is a schematic of composition according to an embodiment of the invention. The composition includes a nucleic acid nanoparticle and a mRNA molecule. The nucleic acid nanoparticle includes a ribosome recruitment functionality, such as an internal ribosome entry site, that enhances translation of the mRNA molecule.

### Internalization of Cargo Molecules Into Cells

A key obstacle in using nucleic acid nanoparticles to deliver therapeutic and diagnostic agents is getting the cargo inside the target cell. The plasma membrane is an amphipathic, phospholipid-containing bilayer that forms a barrier to entry for nucleic acid nanoparticles, which are large, negatively-charged macromolecule complexes. The invention solves this problem by using an element that contains nucleic acid modifications that promote internalization of the cargo into cells. The element may be attached directly to the cargo molecule, the element may be linked to the cargo molecule via a nanoparticle, such as a nucleic acid nanoparticle, or the element and the nanoparticle may be independently attached to the cargo molecule.

Several types of modifications of nucleic acid nanoparticles can be used to promote internalization of cargo molecules, such as those described below. The element that promotes internalization may contain one or more such modifications.

One type of internalization-promoting modification is the addition of chemical moieties to the 2′ position of nucleotides of RNA molecules in the nucleic acid nanoparticle. For example, nucleotides of RNA molecules may contain any of the 2′ modifications listed above in relation to nucleic acid nanoparticles. The 2′ modifications may be restricted to a particular type of nucleotide, such as pyrimidines, purines, adenosines, cytosines, guanosines, thymines, or uridines. The moieties attached to the 2′ position of nucleotides may be hydrophobic, such as fluoro-containing moieties or methoxyethyl. In addition to promoting cellular uptake, such modifications may increase nuclease resistance, thermal stability, and half-life in tissues. See, e.g., Janas MM, et al., Impact of Oligonucleotide Structure, Chemistry, and Delivery Method on In Vitro Cytotoxicity, Nucleic Acid Ther. 2017 Feb;27(1):11-22. doi: 10.1089/nat.2016.0639, the contents of which are incorporated herein by reference. Another 2′ substituent that may be used is N-(aminoethyl)carbamoyl)methyl; the nucleotide 2′-O-(N-(aminoethyl)carbamoyl)methyl adenosine has nuclease-resistance and cell-penetrating properties. Milton S, et al., Nuclease resistant oligonucleotides with cell penetrating properties, Chem Commun (Camb). 2015 Mar 7;51(19):4044-47. doi: 10.1039/c4cc08837a. Alternatively or additionally, the 2′ substituent may be a positively-charged moiety, such as a guanidyl group or a cell-penetrating peptide. The introduction of positive charges into oligonucleotides to reduce the overall negative charge facilitates interaction with the negatively charged cell membrane during cell penetration and avoids having zero-surface charge, which leads to decreases in cellular uptake. See, e.g., Kettler K, et al., Cellular uptake of nanoparticles as determined by particle properties, experimental conditions, and cell type, Environ Toxicol Chem. 2014 Mar;33(3):481-92. doi: 10.1002/etc.2470, the contents of which are incorporated herein by reference.

Another type of modification of nucleic acids in a nanoparticle that can be used to promote internalization into cells is to alter the phosphate backbone. For example, the substitution of phosphorothioate linkages for some or all of the phosphodiester linkages promotes cellular uptake due to higher binding affinity to the cell surface. Such modification also increase resistance to nucleases and promote metabolic stability. Janas MM, et al., Impact of Oligonucleotide Structure, Chemistry, and Delivery Method on In Vitro Cytotoxicity, Nucleic Acid Ther. 2017 Feb;27(1):11-22. doi: 10.1089/nat.2016.0639, the contents of which are incorporated herein by reference. Phosphorothioate linkages could be introduced during solid phase synthesis to produce a mixture of diastereomers or using P(V)-based phosphoramidites to induce stereo control during solid phase synthesis. See Krouse KW, et al., Unlocking P(V): Reagents for chiral phosphorothioate synthesis, Science 2018 Sep 21;361(6408):1234-1238, doi: 10.1126/science.aau3369, the contents of which are incorporated herein by reference. The nucleic acids of the nanoparticle may contain any of the phosphorus-containing backbones or linkages described above.

The element that promotes internalization may contain one or more of the following modifications: diselenolane, 3-(bis(4-methoxyphenyl)(phenyl)methoxy)propyl (3-(tert-butyldisulfaneyl)propyl) diisopropylphosphoramidite, 3-(bis(4-methoxyphenyl)(phenyl)methoxy)propyl ((4S,5S)-5-ethoxy-1,2-dithian-4-yl) diisopropylphosphoramidite, N-(2-thioureidoethyl)-1,2-dithiolane-4-carboxamide, 5-(1,2-dithiolan-3-yl)-N-(2-thioureidoethyl)pentanamide, O-(5-hydroxy-1,2-dithian-4-yl) carbamothioate, 5-((2-acetamido-3-methoxy-3-oxopropyl)disulfaneyl)-2-nitrobenzoic acid, and alkyl phosphorothioate.

The element that promotes internalization may contain nucleotides that have other modifications to the sugar and/or base. For example, the element may contain any of the modified nucleotides described above in relation to nucleic acid nanoparticles.

### Intracellular Targeting of Cargo Molecules

Another technical challenge of nucleic acid nanoparticle-based delivery of cargo is achieving delivery of the cargo to right intracellular destination. For example, as discussed above, some therapeutic RNA molecules, including mRNAs and inhibitory RNAs, such as lnRNA, miRNA, shRNA, and siRNA, need to be exported to the cytoplasm to exert their effects. Other mRNAs are translated on ribosomes at the endoplasmic reticulum. Therapeutic DNAs, on the other hand, must enter the nucleus. Moreover, targeting to those and other sites is often predicated on escape of the cargo from endosomal vesicles. The invention addresses the issue of intracellular targeting generally by using an element that promotes targeting of cargo molecules to an intracellular destination. The element may be attached directly to the cargo molecule, the element may be linked to the cargo molecule via a nanoparticle, such as a nucleic acid nanoparticle, or the element and the nanoparticle may be independently attached to the cargo molecule.

For example and without limitation, the element may be or contain one or more of the following: a nuclear localization signal; an aptamer that facilitates delivery to the cytoplasm; and an endosomal escape mechanism. Nuclear localization signals are known in the art and described in, for example, Sun Y, et al., Factors influencing the nuclear targeting ability of nuclear localization signals, J Drug Target. 2016 Dec;24(10):927-933, doi: 10.1080/1061186X.2016.1184273, the contents of which are incorporated herein by reference. Endosomal escape mechanisms are described in more detail below.

### Endosomal Escape of Cargo Molecules

As discussed above, intracellular targeting of cargo molecules is a key challenge in developing compositions that use nucleic acid nanoparticles to deliver therapeutic or diagnostic cargo. In particular, nanoparticle-containing compositions that are internalized by cells often become trapped in endosomes. The invention addresses this problem specifically by providing compositions that include one or more elements that promote escape of the cargo molecule from endosomes or related compartments. The element may be attached directly to the cargo molecule, the element may be linked to the cargo molecule via a nanoparticle, such as a nucleic acid nanoparticle, or the element and the nanoparticle may be independently attached to the cargo molecule.

Endocytosis involves the budding of vesicles from the plasma membrane and routing of the vesicles to the lysosome, where the endocytosed cargo is degraded. The pH within endosomes decreases en route to the lysosome, and the acidic environment of the lysosome supports degradation of macromolecules. Consequently, the use of pH-sensitive linkers to join cargo molecules to nucleic acid nanoparticles allows secure attachment in the neutral pH of the circulating blood or other extracellular milieus but release of the cargo from the nanoparticle in the vesicles endocytic pathway. See, e.g, Gujrati M, et al., Multifunctional pH-Sensitive Amino Lipids for siRNA Delivery, Bioconjug Chem. 2016 Jan 20;27(1):19-35, doi: 10.1021/acs.bioconjchem.5b00538, the contents of which are incorporated herein by reference.

One approach to promote endosomal escape is to modify nucleotides and/or conjugate small molecules having particular properties to nucleotides in a RNA nanoparticle. The modification or addition may be at the 2′ position of a nucleotide, the base of nucleotide, or the phosphorus-containing backbone. For example and without limitations, the conjugates may be or include the following: 2′-O-imidazolacetyl modification, 2′-O-[N,N dimethylaminoethoxy]ethyl modification, alkyl-phosphorothioates, amines (e.g., a combination of primary, secondary, tertiary, and imidazole amines with different pKₐ values), cholesterol lipids, endosomal enhancing domains, fluorinated alkyne chains, guanidinobutylphosphoramidate, hydrophobic groups, positively-charge moieties, triethylene glycol, and trifluormethylquinoline. Such modifications have been used to facilitate endosomal escape of other types macromolecules. For example, hydrophobic amino acids such as tryptophan or phenylalanine can enhance endosomal escape due to the interaction and pore formation with the endosomal membrane. Amines, including combinations of primary, secondary, tertiary, and imidazole amines with different pKa values, are protonated at pH 5-7 and promote endosomal escape by rupturing the particle membrane and releasing the cargo into the cytosol. Many of the aforementioned strategies to promote endosomal escape are described in, for example, Liu D and Auguste DT, Cancer targeted therapeutics: From molecules to drug delivery vehicles, J Control Release. 2015 Dec 10;219:632-643. doi: 10.1016/j.jconrel.2015.08.041; Singh DD, et al., CRISPR/Cas9 guided genome and epigenome engineering and its therapeutic applications in immune mediated diseases, Semin Cell Dev Biol. 2019 Jun 19. pii: S1084-9521(18)30111-3. doi: 10.1016/j.semcdb.2019.05.007; and Lonn P, et al., Enhancing Endosomal Escape for Intracellular Delivery of Macromolecular Biologic Therapeutics, Sci Rep. 2016 Sep 8;6:32301. doi: 10.1038/srep32301; Gujrati M, et al., Multifunctional pH-Sensitive Amino Lipids for siRNA Delivery, Bioconjug Chem. 2016 Jan 20;27(1):19-35, doi: 10.1021/acs.bioconjchem.5b00538; Deglane, G., et al., Impact of the guanidinium group on hybridization and cellular uptake of cationic oligonucleotides, Chembiochem, 2006 7(4): p. 684-92, DOI: 10.1002/cbic.200500433; Prhavc, M., et al., 2′-O-[2-[2-(N,N-dimethylamino)ethoxy]ethyl] modified oligonucleotides: symbiosis of charge interaction factors and stereoelectronic effects, Org Lett, 2003. 5(12): p. 2017-20, DOI: 10.1021/o10340991; Shen, W., et al., Journal of Materials Chemistry B, 2016. 4(39): p. 6468-6474; and Kurrikoff, K, et al., Recent in vivo advances in cell-penetrating peptide-assisted drug delivery, Expert Opin Drug Deliv, 2016, 13(3): p. 373-87, DOI: 10.1517/17425247.2016.1125879; Gilleron J, et al., Image-based analysis of lipid nanoparticle-mediated siRNA delivery, intracellular trafficking and endosomal escape, Nat Biotechnol. 2013 Jul;31(7):638-46. doi: 10.1038/nbt.2612, the contents of each of which are incorporated herein by reference. The element that promotes endosomal escape may contain any of the modified nucleotides described above in relation to nucleic acid nanoparticles.

The element that promotes endosomal escape may be or contain one or more of the following molecules: UN 7938, trifluormethylquinoline, UN 2383, CPW1F10, CBN40D12, ADD41 D14, ADD29 F15, CBN40H10, CBN40 K7, BADGE, CBN35 C21, CBNO53 M19, CPW1-J18, methoxychlor, CPW097 A20, LOMATIN, guanabenz, UNC7938, CPM2, 3-(perfluorobut-1-yl)-1-hydroxypropyl, 3-(perfluorohex-1-yl)-1-hydroxypropyl, 3-deazapteridine, α-tocopherol, verapamil, nigericin, 3,3,4,4,5,5,6,6,7,7,8,8,9,9,9-pentadecafluorononan-2-one, or N1-ethyl-N1-methyl-N2-(7-(trifluoromethyl)quinolin-4-yl)ethane-1,2-diamine, TfR-T₁₂, melittin, HA2, folate, octa-arginine conjugate stearyl-R8, a locked nucleic acid, a peptide transduction domain, and a fluorinated or perfluorinated compound.

In some embodiments, the element that promotes endosomal escape has a guanidinobutylphosphoramidate, as shown below:

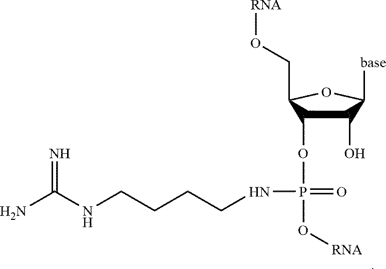

In some embodiments, the element that promotes endosomal escape has a 2′-O-imidazolacetyl modification, as shown below:

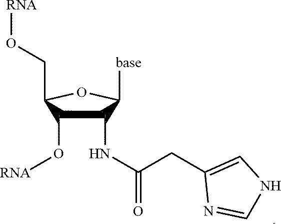

In some embodiments, the element that promotes endosomal escape has a 2′-O-[N,N-dimethylamino)ethoxy]ethyl modification, as shown below:

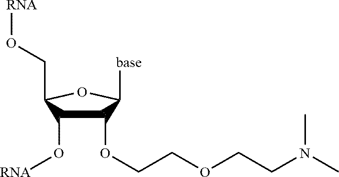

In some embodiments, the element that promotes endosomal escape has a dsDNA with cholesterol lipid conjugated via a triethylene glycol (TEG) linker attached as an arm on the core NP.

In some embodiments, the element that promotes endosomal escape has cholesterol attached directly to a molecule of RNA.

In some embodiments, the element that promotes endosomal escape has a hydrophobic belt of alkyl-phosphorothioates (PPT) attached to a dsDNA or dsRNA.

In some embodiments, the element that promotes endosomal escape contains or is a component having a pKₐ of from about 5.0 to about 7.0.

In some embodiments, the element that promotes endosomal escape contains or is a hydrophobic component.

In some embodiments, the element that promotes endosomal escape contains or is a component that is positively-charged at a pH of about 7.0.

In some embodiments, the element that promotes endosomal escape contains or is a peptide. The peptide may include one or more of the sequences in Table 1.

TABLE 1

| SEQ ID NO. | Sequence | SEQ ID NO. | Sequence | SEQ ID NO. | Sequence |
|---|---|---|---|---|---|
| 1 | GWWG | 21 | HCLLL | 41 | CHGWYWMDLLL |
| 2 | CHGWWG | 22 | HLL | 42 | GWYWMDLLL |
| 3 | CHGWWGLLL | 23 | CGFWFGLLL | 43 | FFLIPKG |
| 4 | GWWGLLL | 24 | HGFWFGLLL | 44 | CFFLIPKG |
| 5 | CGWWGLLL | 25 | CHGFWFGLLL | 45 | HCFFLIPKG |
| 6 | HCGWWGLLL | 26 | HCGFWFGLLL | 46 | CHFFLIPKG |
| 7 | HGWWGLLL | 27 | GFWFGLLL | 47 | HFFLIPKG |
| 8 | CGWWG | 28 | GFWFG | 48 | FFLIPKGLLL |
| 9 | HGWWG | 29 | CGFWFG | 49 | CFFLIPKGLLL |
| 10 | CGFWFGLLL | 30 | CHGFWFG | 50 | HCFFLIPKGLLL |
| 11 | GFWFGLLL | 31 | HCGFWFG | 51 | CHFFLIPKGLLL |
| 12 | HCGFWFGLLL | 32 | HGFWFG | 52 | HFFLIPKGLLL |
| 13 | HGFWFGLLL | 33 | GWYWMDL | 53 | HYF |
| 14 | CGFWFG | 34 | CGWYWMDL | 54 | CHYF |
| 15 | HGFWFG | 35 | HGWYWMDL | 55 | HCHYF |
| 16 | GFWFG | 36 | HCGWYWMDL | 56 | CHHYF |
| 17 | HCGFWFG | 37 | CHGWYWMDL | 57 | HYFLLL |
| 18 | HCGWWG | 38 | CGWYWMDLLL | 58 | CHYFLLL |
| 19 | CLLL | 39 | HCGWYWMDLLL | 59 | HHYFLLL |
| 20 | LLL | 40 | HGWYWMDLLL | 60 | HCHYFLLL |

In some embodiments, the element that promotes endosomal escape has a hydrophobic moiety, a hydrophilic moiety, and a nucleotide attachment moiety. For example and without limitation, the component may have the following structure:

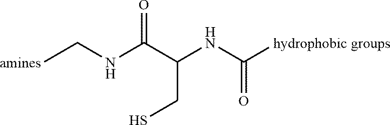

The hydrophilic moiety may include an amine. The hydrophilic moiety may be spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(Hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, or a β-aminoester.

The nucleotide attachment moiety may be cysteine.

The element may promote endosomal escape of the cargo molecule in a receptor independent-manner. The element may promotes endosomal escape of the cargo molecule in a receptor dependent-manner. The element may bind to a receptor in the cell. The element may contain folate, TfR-T₁₂, or a hemagglutinin peptide.

The composition may contain any of the nucleic acid nanoparticles described above. The nucleic acid nanoparticles may contain any of the modified nucleotides described above.

The composition may contain any of the linkers described above.

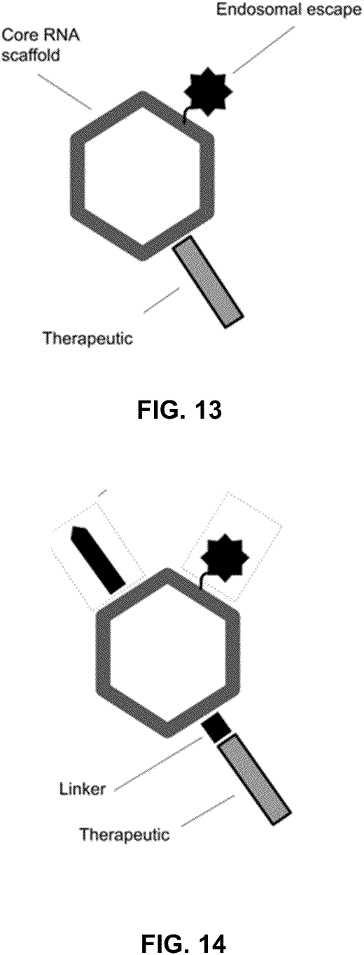

FIG. 13 is a schematic of a composition according to an embodiment of the invention. The composition includes a nucleic acid nanoparticle (core RNA scaffold), cargo molecule (therapeutic), and endosomal escape element.

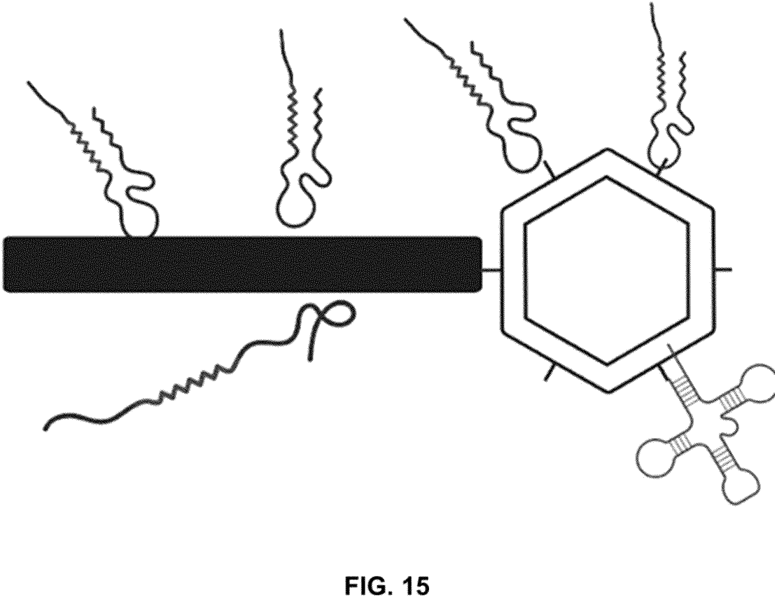

FIG. 14 is a schematic of a composition according to an embodiment of the invention. The composition includes a nucleic acid nanoparticle, cargo molecule (therapeutic), linker, targeting ligand, and endosomal escape element.

FIG. 15 is a schematic of a composition according to an embodiment of the invention. The composition includes a nucleic acid nanoparticle (hexagon), cargo molecule (black rectangle), and endosomal escape elements (grey cross and blue-purple structures containing sawtooth patterns). The same schematic can be used to represent other embodiments of the invention by replacing the endosomal escape elements with, for example, elements that promote cellular internalization or elements that promote targeting of a the cargo molecule to an intracellular destination.

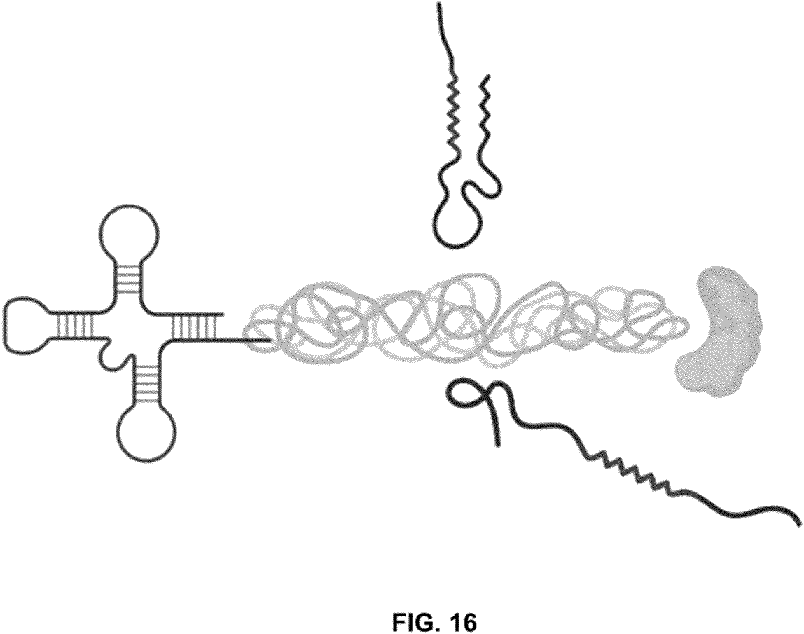

FIG. 16 is a schematic of a composition according to an embodiment of the invention. The composition includes a serum protein (green shape), cargo molecule (blue string-like structure), and endosomal escape elements (red cross and blue-purple structures containing sawtooth patterns). The same schematic can be used to represent other embodiments of the invention by replacing the endosomal escape elements with, for example, elements that promote cellular internalization or elements that promote targeting of a the cargo molecule to an intracellular destination.

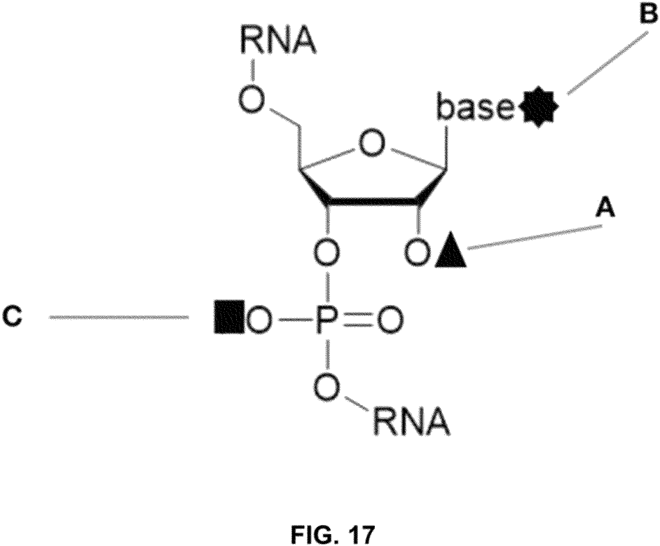

FIG. 17 show sites of modification of a nucleotide in nanoparticles according to an embodiment of the invention. A indicates modification at the 2′ position, B indicated modification of the base, and C indicates the phosphate backbone.

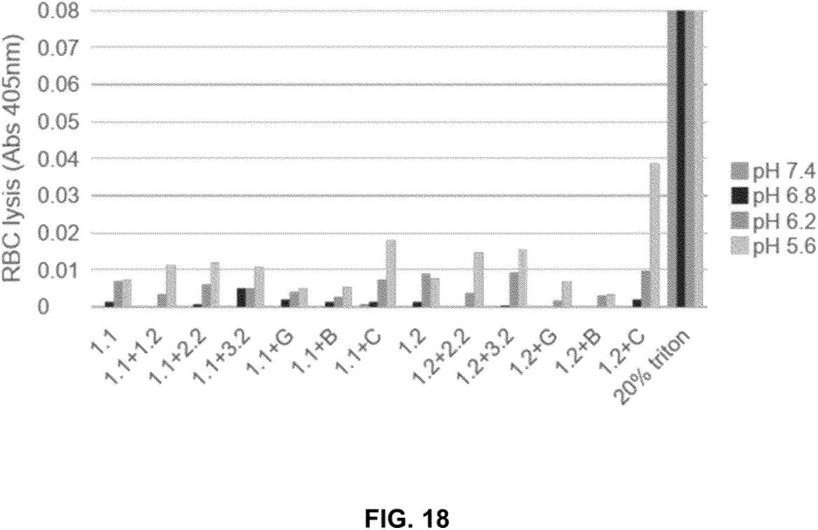

FIG. 18 is graph showing endosomal escape using various compounds alone or in combination. The endosomal escape abilities of compounds alone or in combination with small molecules (G, B, C) were assessed using red blood cells hemolysis assays in 3 different pHs, ranging from physiological (7.4), endosomal (6.5) to lysosomal (5.8). The results were quantified by measuring absorbance at 405 nm. 20% Triton was used as a positive control, and all results are normalized to the negative (water) control.

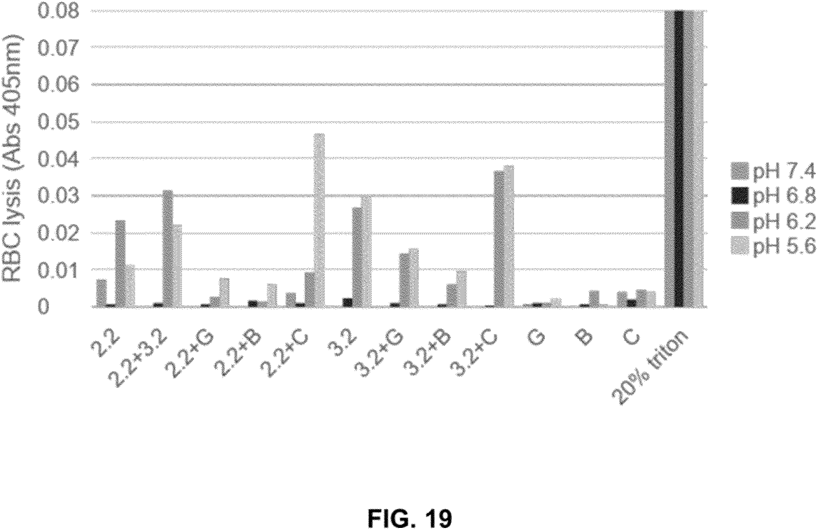

FIG. 19 is graph showing endosomal escape using various compounds alone or in combination. The endosomal escape abilities of compounds alone or in combination with small molecules (G, B, C) were assessed using red blood cells hemolysis assays in 3 different pHs, ranging from physiological (7.4), endosomal (6.5) to lysosomal (5.8). The results were quantified by measuring absorbance at 405 nm. 20% Triton was used as a positive control, and all results are normalized to the negative (water) control.

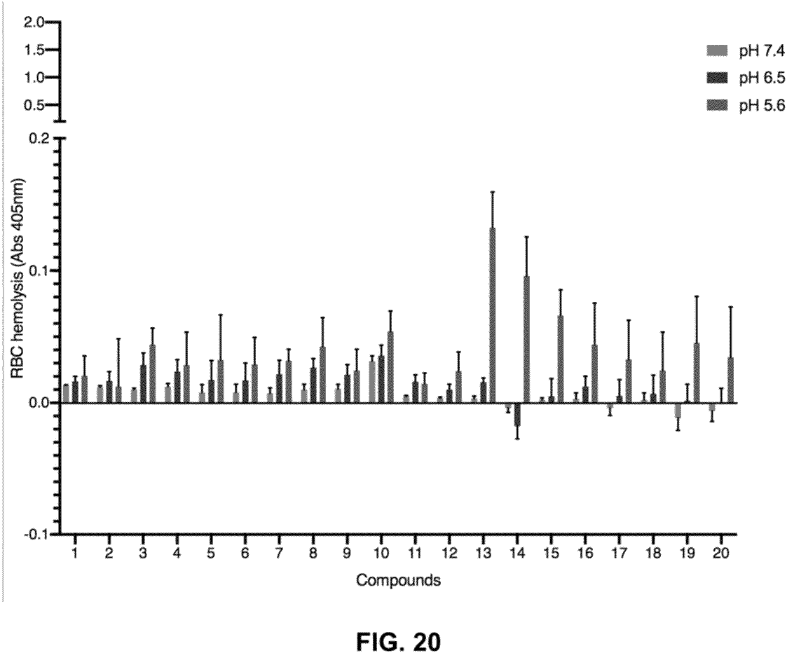

FIG. 20 is a graph showing endosomal escape using various compounds. The endosomal escape abilities of compounds were assessed using red blood cells hemolysis assays in 3 different pHs, ranging from physiological (7.4), endosomal (6.5) to lysosomal (5.8). The results were quantified by measuring absorbance at 405 nm. 20% Triton was used as a positive control, and all results are normalized to the negative (water) control.

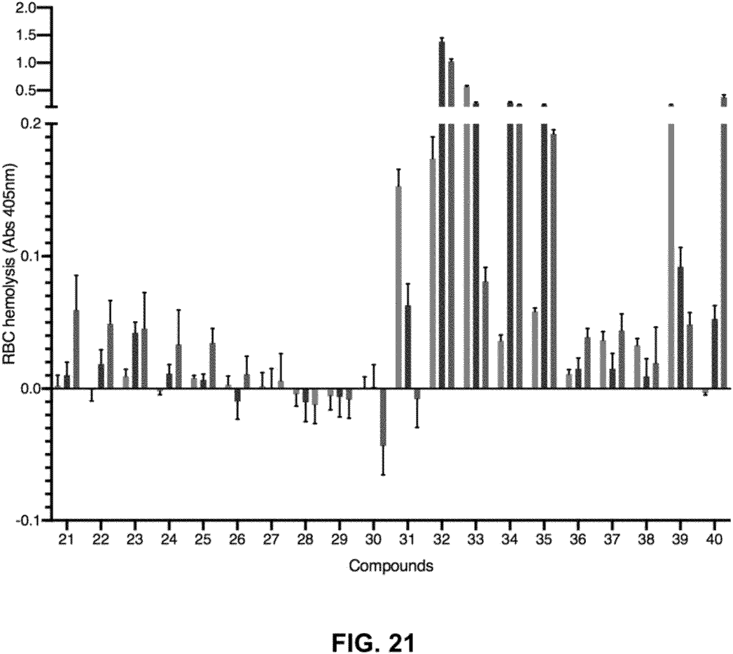

FIG. 21 is a graph showing endosomal escape using various compounds. The endosomal escape abilities of compounds were assessed using red blood cells hemolysis assays in 3 different pHs, ranging from physiological (7.4), endosomal (6.5) to lysosomal (5.8). The results were quantified by measuring absorbance at 405 nm. 20% Triton was used as a positive control, and all results are normalized to the negative (water) control.

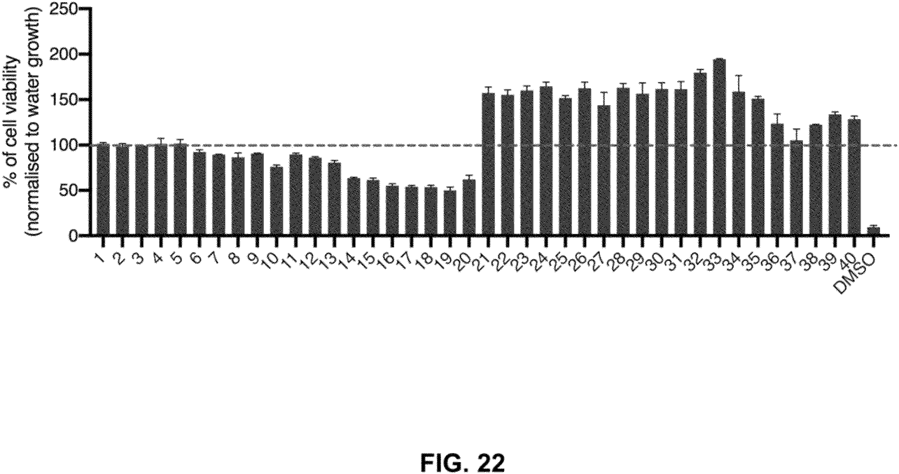

FIG. 22 is a graph showing toxicity effects of endosomal escape compounds. The effects on mammalian cells were assessed using a glioblastoma cell line (VS-GB) after incubation with the compound for 48 hours. Cell viability was measured using the Titer-Cell Glo luminescence assay. The results showed that the majority of the compounds did not exert cytotoxicity.

### Binding of Proteins, Including Plasma Proteins

As indicated above, degradation of cargo molecules, poor internalization of cargo molecules, and entrapment in the endocytic pathway are all obstacles that hinder delivery of therapeutic and diagnostic agents. Other difficulties include cargo toxicity and immunogenicity and directing the cargo to the proper tissue, i.e., tissue tropism. In certain embodiments of the invention, such problems are overcome through the use of an element that promotes or inhibits binding of proteins to the cargo-containing composition. The element may be attached directly to the cargo molecule, the element may be linked to the cargo molecule via a nanoparticle, such as a nucleic acid nanoparticle, or the element and the nanoparticle may be independently attached to the cargo molecule.

The element may bind or repel a specific protein or group of proteins. For example, the element may bind to a protein in the plasma of a subject to form a corona around the cargo-containing composition. The corona may protect the cargo from degradation and thus stabilize the composition in circulation. Alternatively or additionally, the corona may alter surface charge of the composition to minimize charge repulsion that can interfere with cellular uptake. In certain embodiments, the element repels a protein, such as an immunoglobulin, that could bind to the composition to interfere with cargo function. The element may bind a first set of one or more proteins and repel a second set of one or more proteins.

The element may bind to any protein that increases cellular uptake, stability, or another useful property of the nanoparticle. Elements that bind to plasma proteins are particularly useful because such compositions that contain such elements can be injected as naked nanoparticles into the blood, where they quickly become coated. For example and without limitation, the plasma protein may be albumin, fibrinogen, fibronectin, haptoglobin, immunoglobulin, α-1-acidglycoprotein, α1-antitrypsin, α-2-macroglobulin, or α-thrombin. The use of protein-coated nanoparticles is described in, for example, Oh JY, et al., Cloaking nanoparticles with protein corona shield for targeted drug delivery, Nat Commun. 2018 Oct 31;9(1):4548. doi: 10.1038/s41467-018-06979-4; Humphreys SC, Plasma and liver protein binding of GalNAc conjugated siRNA, Drug Metab Dispos. 2019 May 16. pii: dmd.119.086967. doi: 10.1124/dmd. 119.086967; Juliano RL and Carver K, Cellular uptake and intracellular trafficking of oligonucleotides, Adv Drug Deliv Rev. 2015 Jun 29;87:35-45. doi: 10.1016/j.addr.2015.04.005; Elzoghby, AO, et al., Albumin-based nanoparticles as potential controlled release drug delivery systems, J Control Release, 2012 Jan 30;157(2):168-82. doi: 10.1016/j.jconrel.2011.07.031; and Misak, HE, Albumin-based nanocomposite spheres for advanced drug delivery systems, Biotechnol J. 2014 Jan;9(1):163-70, the contents of each of which are incorporated herein by reference.

Any mechanism may be used to promote binding of proteins to the element. For example, the element may contain nucleic acids that contain altered phosphorus-containing backbones, such as phosphorothioate or any of the other linkages described above. Altered backbones change the charge of a nucleic acid and thus the binding of plasma or serum proteins to the nucleic acid. The element may contain one or more ligands, such as aptamers, that bind specific plasma or serum proteins. When the element is a component of a nucleic acid nanoparticle, modifications of the shape or three-dimensional arrangement of nucleic acids that make up the structural core of the nanoparticle may also be used to promote binding of proteins, such as serum or plasma proteins.

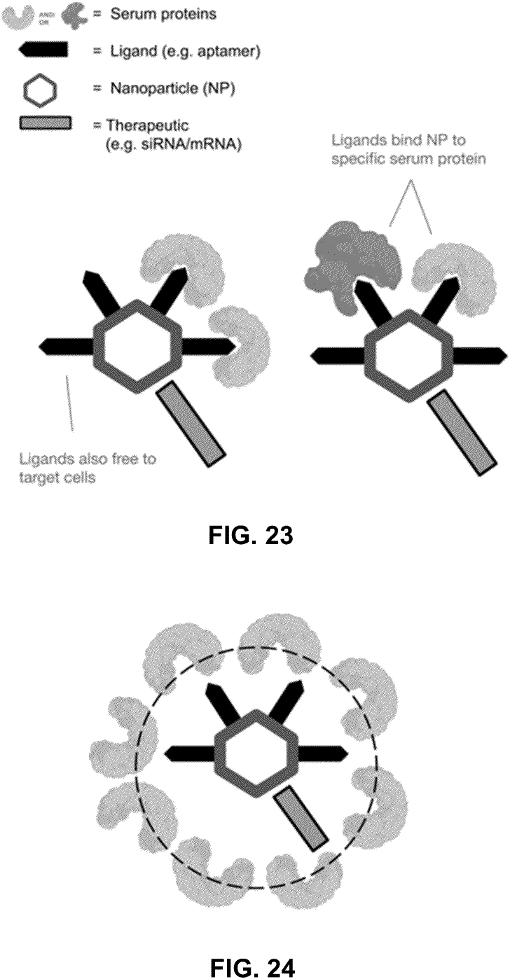

FIG. 23 is a schematic of a composition according to an embodiment of the invention. The composition includes serum proteins (colored shapes), ligands (pentagons), a nucleic acid nanoparticle (hexagon), a therapeutic cargo molecule (rectangle). In the embodiment shown, the serum proteins bind to ligands on the nanoparticle.

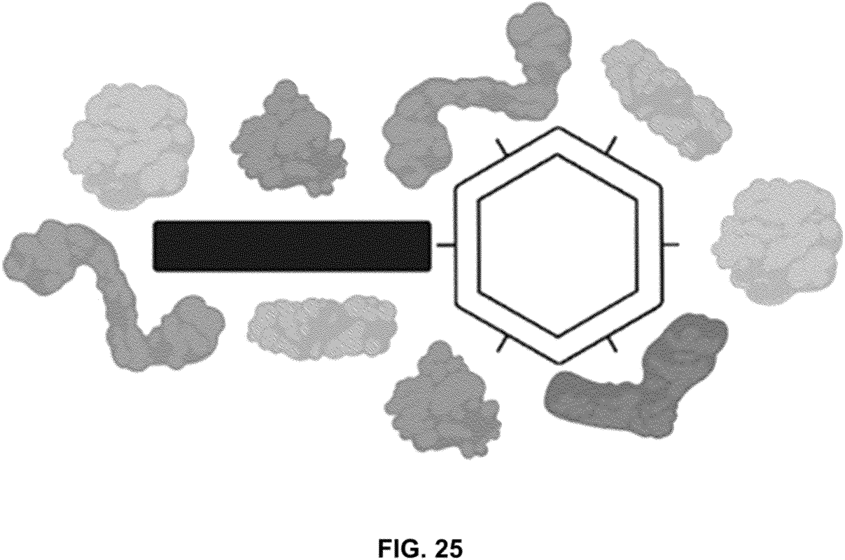

FIG. 24 is a schematic of a composition according to an embodiment of the invention. The composition includes serum proteins (blue shapes), ligands (pentagons), a nucleic acid nanoparticle (hexagon), a therapeutic cargo molecule (rectangle). In the embodiment shown, the serum proteins bind to the nanoparticle via charge interactions.

In embodiments in which the cargo molecule is associated with a nanoparticle, the element can be configured so that the corona forms over the cargo molecule only, over the nanoparticle only, or over both. In embodiments in which the composition comprises only the element conjugated to the cargo molecule, the corona forms around the cargo molecule.

FIG. 25 is a schematic of a composition according to an embodiment of the invention. The composition includes serum proteins (colored shapes), a nucleic acid nanoparticle (hexagon), a therapeutic cargo molecule (black rectangle). In this embodiment, the serum proteins form a corona that surrounds the nanoparticle and cargo molecule.

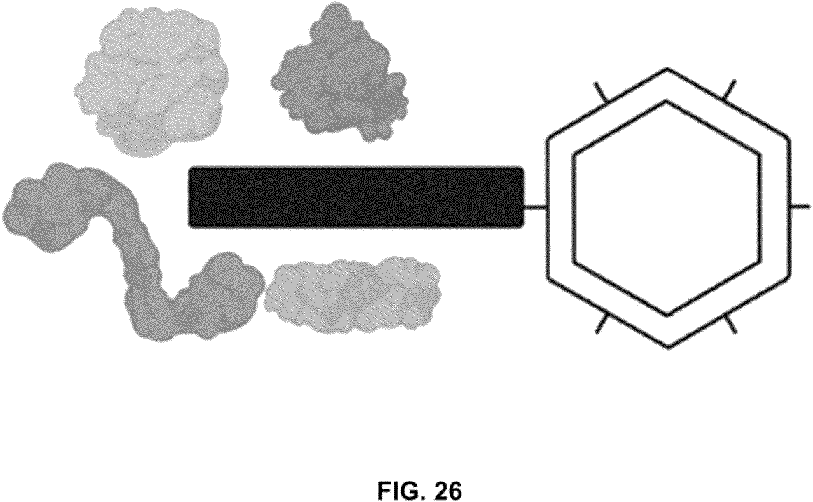

FIG. 26 is a schematic of a composition according to an embodiment of the invention. The composition includes serum proteins (colored shapes), a nucleic acid nanoparticle (hexagon), a therapeutic cargo molecule (black rectangle). In this embodiment, the serum proteins form a corona that surrounds only the cargo molecule.

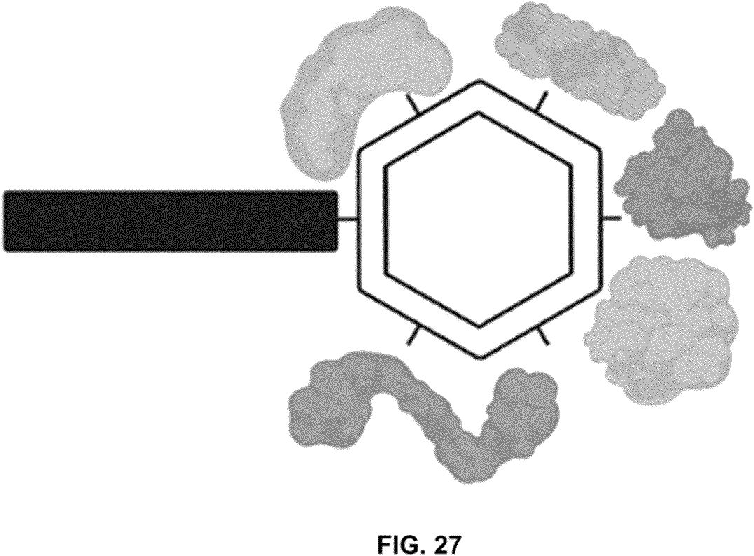

FIG. 27 is a schematic of a composition according to an embodiment of the invention. The composition includes serum proteins (colored shapes), a nucleic acid nanoparticle (hexagon), a therapeutic cargo molecule (black rectangle). In this embodiment, the serum proteins form a corona that surrounds only the nanoparticle.

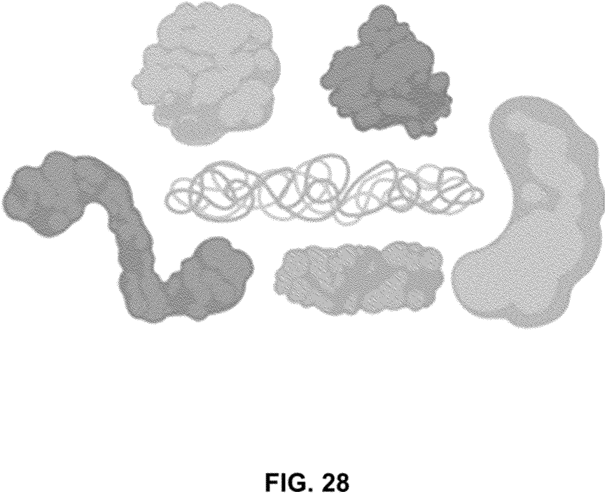

FIG. 28 is a schematic of a composition according to an embodiment of the invention. The composition includes serum proteins (colored shapes) and a therapeutic cargo molecule (blue string-like structure). In this embodiment, the serum proteins form a corona around the cargo molecule.

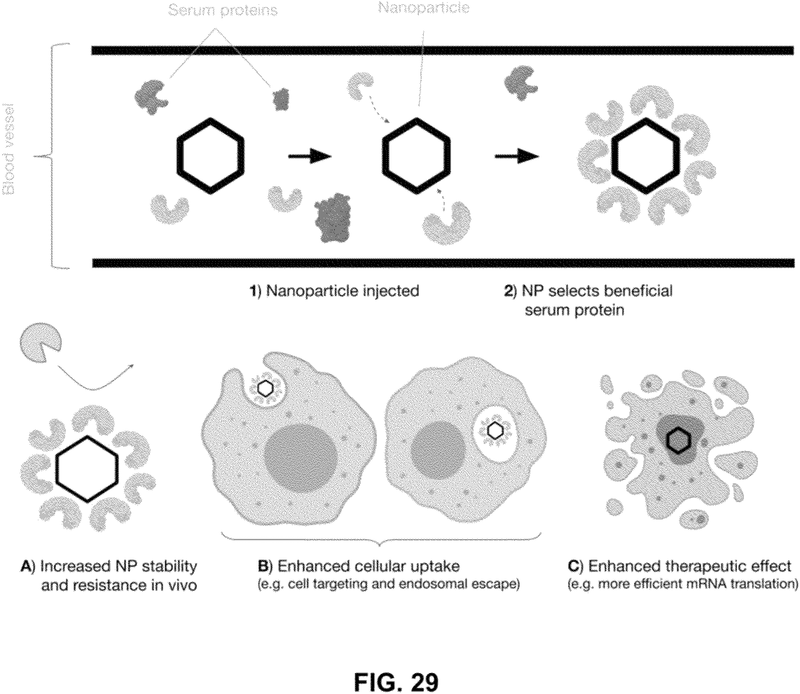

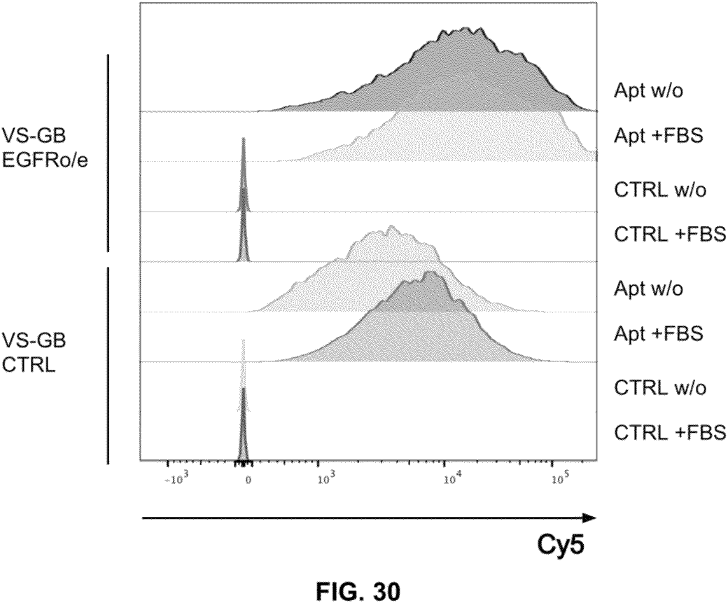

FIG. 29 is a schematic showing activity of composition according to an embodiment of the invention. The composition includes serum proteins (colored shapes), a nucleic acid nanoparticle (hexagon), and a therapeutic cargo molecule (not shown). When the nanoparticle is injected into the blood, it becomes coated with a serum protein, which forms a corona, as shown in the upper portion of the figure. The corona protects the nanoparticle from nuclease digestion to increase nanoparticle stability (A, lower left) and enhances cellular uptake (B, lower center). The nanoparticle includes additional functionality, such as increased translation of a cargo mRNA molecule, to enhance the therapeutic effect of the cargo molecule (C, lower right).

FIG. 30 is a graph showing the effect of a protein corona on cellular uptake of RNA nanoparticles. Aptamers labelled with a Cy5 fluorescent tag were incubated for 2 hours with VS-GB cells in media with (FBS) and without serum (w/o). Flow cytometry was used to determine the cell-internalization abilities of aptamers. The results show that the presence of serum proteins enhanced cellular internalization.

### EXAMPLES

### Example 1

The naming format for constructed used herein is provided in Table 2.

TABLE 2

| | | |
|---|---|---|
| S1- | TTUO- | 001 |
| S = construct shape | T = targeting | Version (specific attachments listed in a separate key) |
| 1 = design iteration | T = therapy | |
| | U = uptake | |
| | O = other | |
| | A number is assigned to outline how many of these modifications are present | |

Exemplary RNA monomers used to form RNA nanoparticles described herein are provided in Table 3.

TABLE 3

| | | |
|---|---|---|
| Identifier | Sequence | Modifications/comments |
| C-1.0 | GGGAAAcucuGucGuGGGAcGGucAGAcuGuucAAccAcuccucuuc | 2′F U, C |
| C-1.1 | [Thiol C6 S-S] GGGAAAcucuGucGuGGGAcGGucAGAcuGuucAAccAcuccucuuc | 2′F U, C 5′ Thiol C6 S-S modifier |
| C-2.0 | GGGAAAGAAGAGGAGuGGAcGGuAcuGuGuuucAAccuGucucuGAc | 2′F U, C |
| C-2.1 | [Thiol C6 S-S] GGGAAAGAAGAGGAGuGGAcGGuAcuGuGuuucAAccuGucucuGAc | 2′F U, C 5′ Thiol C6 S-S modifier |
| C-3.0 | GGGAAAGcAGuGuAGcGGAcGGuGuGucAGuucAAcccAcGAcAGAG | 2′F U, C |
| C-3.1 | cAGuGuccGAuAuAcGcucGGGGAAAGcAGuGuAGcGGAcGGuGuGucAGuucAAcccAcGAcAGAG | 2′F U, C [additional region for hybridization] |
| C-4.0 | GGGAAAGucAGAGAcAGGAcGGucuAGGucuucAAccGcuAcAcuGc | 2′F U, C |
| C-4.1 | [Thiol C6 S-S] GGGAAAGucAGAGAcAGGAcGGucuAGGucuucAAccGcuAcAcuGc | 2′F U, C 5′ Thiol C6 S-S modifier |
| C-5.0 | GGGAAAcuAGAuuGGAAcAcAGuAuuGGAcAGucuGAuuGGAcuGAcAcAuuGGAGAc | 2′F U, C |
| C-5.1 | [Cy5] GGGAAAcuAGAuuGGAAcAcAGuAuuGGAcAGucuGAuuGGAcuGAcAcAuuGGAGAc | 2′F U, C 5′ Cy5 |
| C-5.2 | [Cy3] GGGAAAcuAGAuuGGAAcAcAGuAuuGGAcAGucuGAuuGGAcuGAcAcAuuGGAGAc | 2′F U, C 5′ Cy3 |

Exemplary siRNAs used herein are provided in Table 4.

TABLE 4

| | | |
|---|---|---|
| Identifier | Sequence | Modifications/comments |
| S-1.0 | GCAAUUACAUGAGCGAGCATT | N/A [sense strand, PLK1-targeting canonical siRNA] |
| S-1.1 | [Thiol C6 S-S] GcAAuuAcAuGAGcGAGcATT | 2′F U, C 5′ Thiol C6 S-S modifier [sense strand, PLK1-targeting canonical siRNA] |
| S-2.0 | UGCUCGCUCAUGUAAUUGCGG | N/A [antisense strand, PLK1-targeting canonical siRNA] |
| S-2.1 | uGcucGcucAuGuAAuuGcGG | 2′F U, C [antisense strand, PLK1-targeting canonical siRNA] |
| S-3.0 | GCAAUUACAUGAGCGAGCACUUGTT | N/A [sense strand, PLK1-targeting dicer substrate siRNA] |
| S-4.0 | AACAAGUGCUCGCUCAUGUAAUUGCGG | N/A [antisense strand, PLK1-targeting dicer substrate siRNA] |
| S-5.0 | 5′pAGAuCACCCuCCUuAAAuAUUAATT | 5′ phosphate, 2′-O-methylation [sense strand, PLK1-targeting Dicer substrate siRNA] |
| S-6.0 | aauuAAUAUUUAAgGAGGGUGAuCUTT | 2′-O-methylation [antisense strand, PLK1-targeting Dicer substrate siRNA] |

Exemplary aptamers used herein are provided in Table 5.

TABLE 5

| | | |
|---|---|---|
| Identifier | Sequence | Modifications/comments |
| A-1.0 | [Cy5] GGAcGGAuuuAAucGccGuAGAAAAGCAuGucAAAGccGGAAccGucc | 2′F U, C 5′ Cy5 E07min (SXF1) aptamer |
| A-1.1 | cGAGcGuAuAucGGAcAcuGuuuuuuGGAcGGAuuuAAucGccGuAGAAAAGcAuGucAAAGccGGAAccGucc | 2′F U, C E07min aptamer [additional region for hybridization] |
| A-2.0 | [Cy5] GccuuAGuAAcGuGcuuuGAuGucGAuucGAcAGGAGGc | 2′F U,C 5′ Cy5 CL4 (SXF2) aptamer |

Peptides used herein are provided in Table 6.

TABLE 6

| | | |
|---|---|---|
| Identifier | Sequence | Modifications/comments |
| P-1.0 | GFWFG | None |
| P-1.1 | GFWFG | SM(PEG)₆ (Pierce) conjugated to N terminus |
| P-1.2 | GFWFG | Maleimide functionalized (via) 6-maleimidohexanoic acid |
| P-2.0 | GFWFG | Double branching (ethylene diamine) |
| P-2.1 | GFWFG | Double branching (ethylene diamine), SM(PEG)₆ conjugated to branching head group |

RNA strands were synthesized using a H-16 synthesizer (K&A). Syntheses were performed on a 1 µmol column using a standard RNA coupling protocol (360 s for 2′-tertbutyldimethylsilyl (TBDSM)-protected amidites and all other modifications. The solutions of amidites, tetrazole and acetonitrile were dried over activated molecular sieves (4 Å) overnight. After synthesis the RNA was deprotected with 1:1 methylamine/ammonium hydroxide (AMA) for 3 h at rt. The solid support was then filtered and washed twice with EtOH:water (1:1) and once with THF. The resultant RNA solution was then evaporated to dryness and dissolved in 200 µl dry DMSO. Then 275 µl TEA*3HF (TREAT-HF) was added and incubated either at 65° C. for 3 h or overnight at room temperature. The RNA was then subjected to EtOH precipitation.

Crude RNA strands were purified on semi-preparative PL-SAX 1000A, 10 µmol 25 x 150 mm column at 60° C. with a flow rate of 10 mL/min and UV detection at 260 nm. Usually, 100-250 µL of crude RNA solution was injected per run. Elution was performed with a linear gradient of approximately Δ10% B over 2 to 2.5 column volumes, with the starting concentration adjusted to the length of the oligonucleotide. Buffer A: 25 mM Tris· HC1, pH 8.0, 20% acetonitrile, 10 mM sodium perchlorate, degassed; Buffer B: 25 mM Tris•HC1, pH 8.0, 20% acetonitrile, 600 mM sodium perchlorate, degassed. Fractions containing RNA were pooled and acetonitrile was removed in vacuo. After dilution with an equal volume of TEAB, the product fractions were loaded onto a C18 SepPak cartridge (Waters, Millipore). The cartridge was washed with water until free of salt and the RNA was eluted with acetonitrile:water 1:1 (v/v). The solution was evaporated, and the RNA dissolved in nuclease-free water for concentration determination by UV absorbance and quality assessment via denaturing PAGE.

### Example 2

The square SQ1 scaffold was assembled according to a standard protocol. Equimolar amounts of the 5 different strands (A, B, C, D, E - FIG. 1A) were combined in PBS + MgC1₂ (2 mM) buffer, with a final concentration of 1 µM. The 5 strands were annealed to each other at 95° C. for 5 min then slowly cooled down to 15° C. The scaffoldwas then analyzed by native polyacrylamide gel electrophoresis (PAGE) and dynamic light scattering (DLS) (vide infra).

For PAGE (FIG. 1B), the assembled scaffold was electrophoresed on native PAGE (6%) in 1X TBMg (890 mM Tris Borate + 20 mM Mg(OAc)₂, pH = 8.3) at a constant voltage of 100 V. Gel bands were visualized using GelRed™. 10 pmol of structures was loaded. 2 µL of glycerin (70% in H2O) was added to samples before loading.

For DLS (FIG. 1C), the assembled scaffold was analyzed using a Malvern Zetasizer Nano S ZEN 1600 Nano Particle Size Analysis - 20 µL of samples were used, and intensity was recorded. Average of three trials was calculated. All measurements were carried out at 25° C. Samples were centrifuged at 12000 rpm for 5 minutes before analysis in order to remove dust and debris.

The design of the square scaffold SQ1 is schematically drawn in FIG. 1A. As shown by PAGE and DLS (FIGS. 1B and 1C), SQ1 assembles into a main clear monodispersed product.

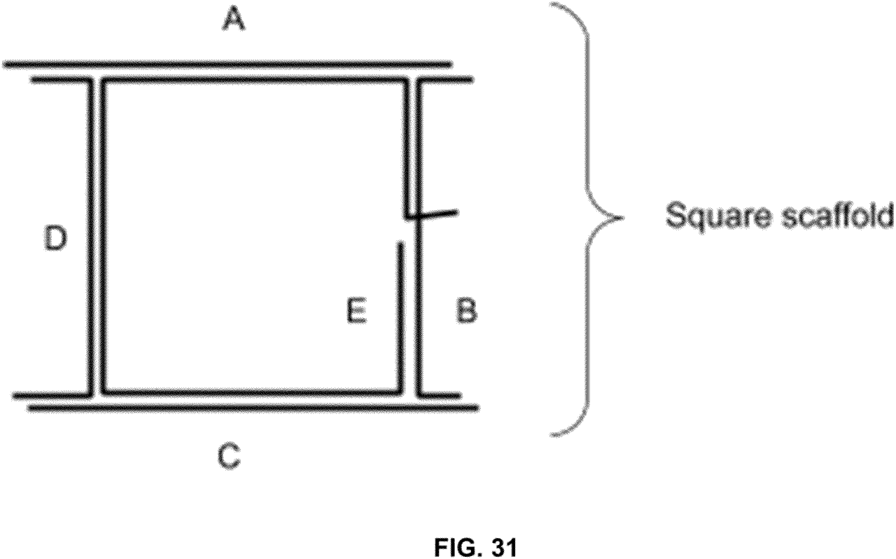

FIG. 31 is a schematic outlining the core construct SQ1-0000-001 used in an embodiment of the invention. These sequences correspond to the following from RNA sequence tables 2-4: A = C-1.01; B = C-2.01; C = C-3.01; D = C-4.01; E = C-5.01.

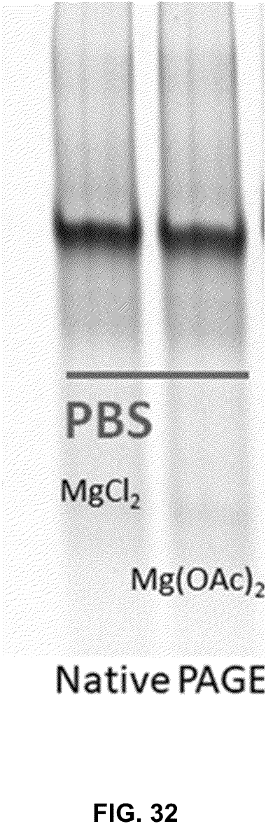

FIG. 32 shows the results of native PAGE of the assembled scaffold SQ1-0000-001.

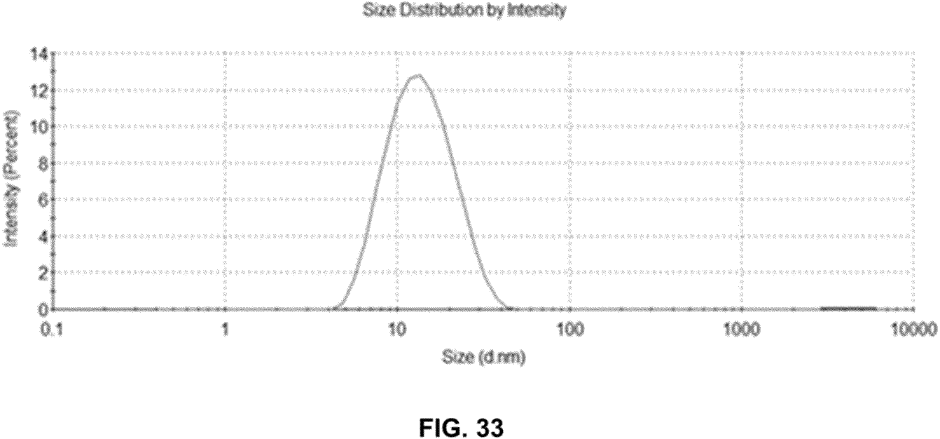

FIG. 33 is a graph showing dynamic light scattering of the SQ1-0000-001 scaffold prior to modification. Measurements were carried out at 25° C.

### Example 3

The SQ1-1210-001 construct is composed of a single aptamer, two siRNAs and one endosomal escape peptide attached to the square scaffold SQ1 (Example 2). It was assembled as follows: Equimolar amounts of the 5 different scaffold strands were combined (C-1.1 coupled to S-1.1, C-2.1 without peptide or C-2.1 with peptide P-1.1, C-3.1, C-4.1 coupled to S-1.1, C-5.1, 2x molar amount of S-2.1) in PBS + Mg(OAc)₂ (2 mM) buffer, with a final construct concentration of 5 µM. Samples were annealed with the following protocol: held at 95° C. for 5 minutes then slowly cooled down to 15° C. (85° C. for 2 minutes, 75° C. for 2 minutes, 65° C. for 2 minutes, 55° C. for 2 minutes, 45° C. for 2 minutes, 35° C. for 2 minutes, 25° C. for 2 minutes, 15° C. for 2 minutes). The aptamer strand (A-1. 1) was heated at 95° C. for 5 minutes, and snap cooled on ice for 5 minutes. It was then added to the assembled core scaffold, and the mix was heated at 50° C. for 10 minutes. The assembled products were loaded on native PAGE (8%) in 1x TBMg at a constant voltage of 100 V. Gel bands were visualized first using the Cy5 channel then using the GelRed™ stain. 10 pmol of construct was loaded. 2 µL of glycerin (70% in water) was added to samples before loading.

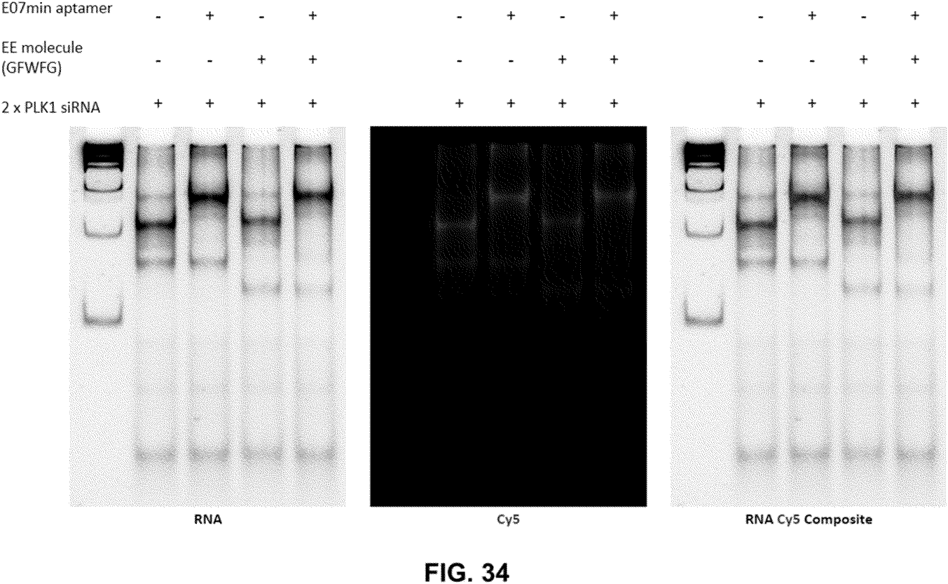

FIG. 34 shows the results of native PAGE of the assembled SQ1-1210-001 construct with Cy5 (C-5.1), the E07min aptamer (A-1.1), endosomal escape-mediating peptide (P-1.1) and 2 x PLK1 siRNAs (S-1.1-S-2.1). The results show that SQ1 constructs functionalized with aptamers, siRNAs and endosomal escape peptides generated a main clear monodispersed product.

### Example 4

RNA molecules with multiple attachment methods were designed, with the aim of being attached to the core construct. The RNA molecule selected was a polo-like kinase 1 (PLK1)-targeting siRNA. Various siRNA designs, differing in target site (FIG. 3A) and attachment chemistries (FIGS. 3B-E) were analysed. siRNA molecules modified with different attachment methods (thiol, alkyne, Dicer substrate) maintained silencing efficiency.

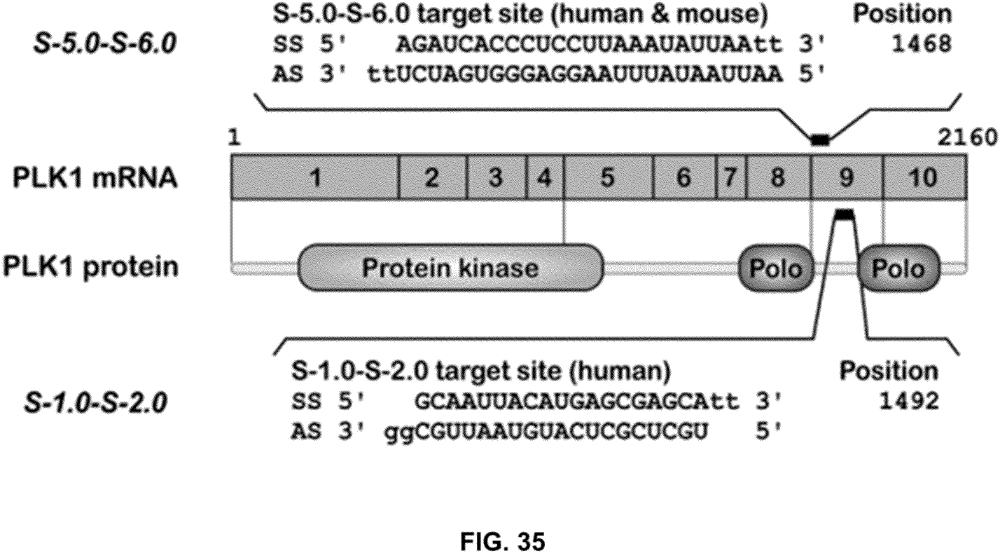

FIG. 35 is a schematic overview over two PLK1 siRNA target sites and their respective siRNA sequences, S-5.0-S-6.0 and S-1.0-S-2.0.

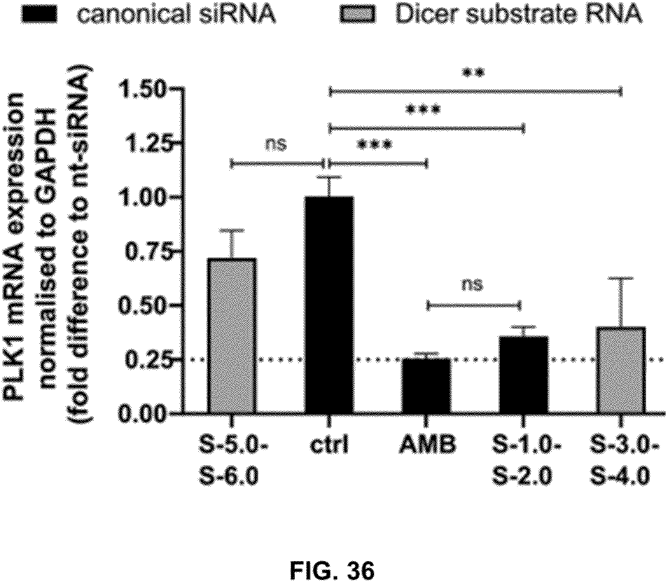

FIG. 36 is a graph showing the knockdown efficacy of three different siRNA designs 72 hours after transfection of HeLa cells with 20 nM siRNA, benchmarked against the commercial SilencerSELECT PLK1 siRNA (AMB). PLK1 transcript levels are expressed relative to cells treated with 20 nM non-targeting siRNA (ctrl) and normalised to GAPDH expression. Error bars represent standard deviations between three technical replicates.

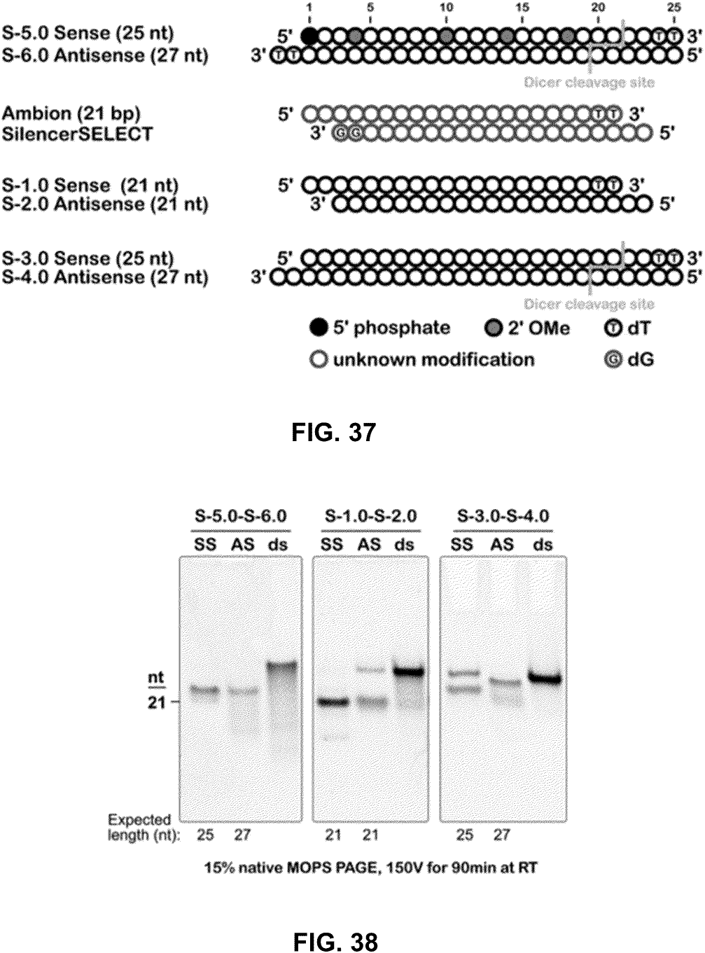

FIG. 37 is a graphic visualization of different canonical (21-bp duplexes with 2-nt 3′ overhangs) and Dicer substrate siRNA designs (25-nt sense and 27-nt antisense strand) that were used in embodiments of the invention. dT and dG indicate DNA nucleotides.

FIG. 38 shows the results of an electrophoretic mobility shift assay to verify siRNA strand annealing. Sense and antisense strands were mixed at equimolar concentrations and subjected to thermal annealing in nuclease-free water.

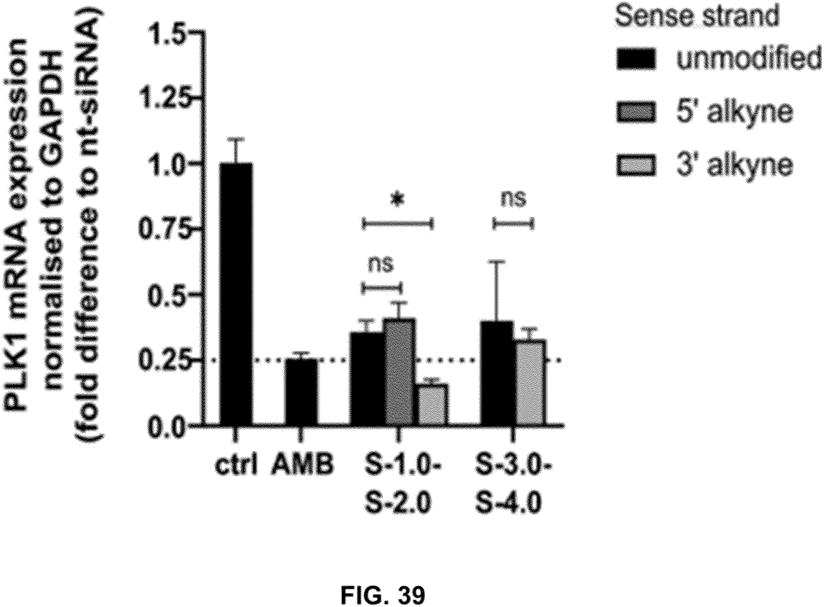

FIG. 39 is graph showing the potency of siRNAs with alkyne-modified sense strands 72 h after transfection of HeLa cells with 20 nM siRNA. PLK1 transcript levels are expressed relative to cells treated with 20 nM non-targeting siRNA (ctrl) and normalised to GAPDH expression. Error bars represent standard deviations between three technical replicates.

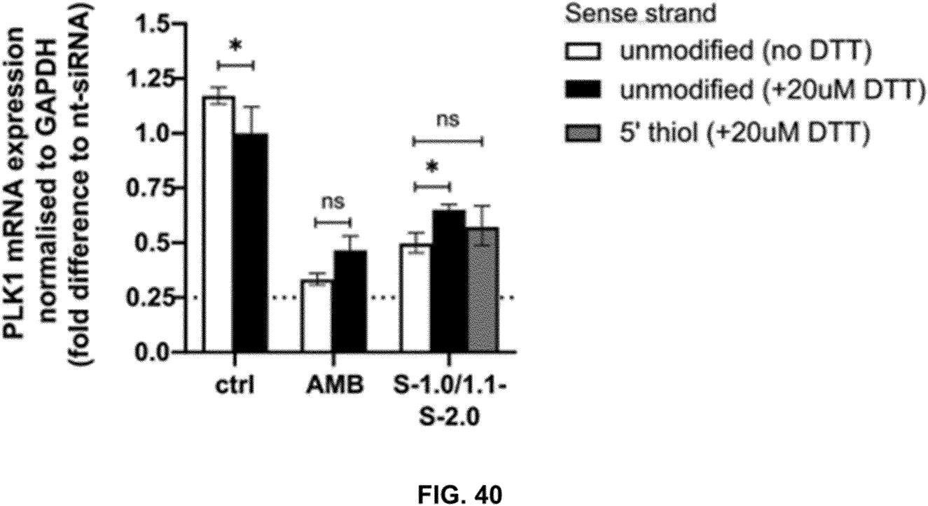

FIG. 40 is graph showing the potency of siRNAs with thiol-modified sense strands 72 h after transfection of HeLa cells with 20 nM siRNA. PLK1 transcript levels are expressed relative to cells treated with 20 nM non-targeting siRNA (ctrl) and normalised to GAPDH expression. Error bars represent standard deviations between three technical replicates.

RNA modifications were introduced onto PLK1 siRNAs to enhance serum stability. siRNA S-1.0-S-2.0 modified with modification patterns 2, 5 and 7 was stable 24-hours post-incubation with human serum and preserved silencing efficiency.

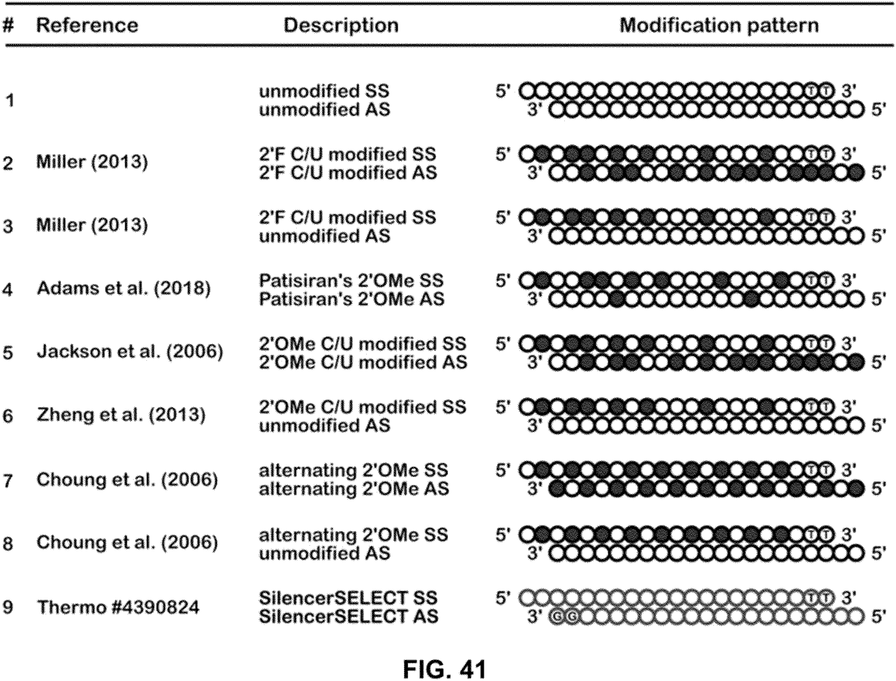

FIG. 41 is a graphic summary of the positioning of 2′-sugar modifications on PLK1 siRNA S-1.0-S-2.0 that were tested for the ability to increase the in vitro stability and potency of PLK1 siRNA. Previous reports in which these modifications were successfully employed are incorporated herein by reference (A.D. Miller, Delivery of RNAi therapeutics: Work in progress, Expert Rev. Med. Devices. (2013); D. Adams, A. Gonzalez-Duarte, W.D. O’Riordan, C.C. Yang, M. Ueda, A. V. Kristen, I. Tournev, H.H. Schmidt, T. Coelho, J.L. Berk, K.P. Lin, G. Vita, S. Attarian, V. Plante-Bordeneuve, M.M. Mezei, J.M. Campistol, J. Buades, T.H. Brannagan, B.J. Kim, J. Oh, Y. Parman, Y. Sekijima, P.N. Hawkins, S.D. Solomon, M. Polydefkis, P.J. Dyck, P.J. Gandhi, S. Goyal, J. Chen, A.L. Strahs, S. V. Nochur, M.T. Sweetser, P.P. Garg, A.K. Vaishnaw, J.A. Gollob, O.B. Suhr, Patisiran, an RNAi therapeutic, for hereditary transthyretin amyloidosis, N. Engl. J. Med. (2018); A.L. Jackson, J. Burchard, D. Leake, A. Reynolds, J. Schelter, J. Guo, J.M. Johnson, L. Lim, J. Karpilow, K. Nichols, W. Marshall, A. Khvorova, P.S. Linsley, Position-specific chemical modification of siRNAs reduces “off-target” transcript silencing, RNA. (2006); M.A. Collingwood, S.D. Rose, L. Huang, C. Hillier, M. Amarzguioui, M.T. Wiiger, H.S. Soifer, J.J. Rossi, M.A. Behlke, Chemical modification patterns compatible with high potency dicer-substrate small interfering RNAs, Oligonucleotides. (2008); J. Zheng, L. Zhang, J. Zhang, X. Wang, K. Ye, Z. Xi, Q. Du, Z. Liang, Single modification at position 14 of siRNA strand abolishes its gene-silencing activity by decreasing both RISC loading and target degradation, FASEB J. (2013); and S. Choung, Y.J. Kim, S. Kim, H.O. Park, Y.C. Choi, Chemical modification of siRNAs to improve serum stability without loss of efficacy, Biochem. Biophys. Res. Commun. (2006)).

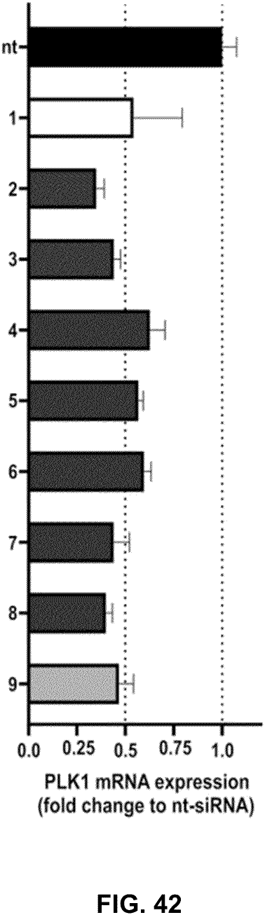

FIG. 42 is a graph of PLK1 expression in transfected MDA-MB-231 cells. Cells were transfected with 20 nM of the indicated siRNAs. After 72 h knockdown was assessed by qPCR. PLK1 expression was normalised to GAPDH expression, relative to cells transfected with 20 nM non-targeting siRNA (nt). Error bars represent standard deviations between two technical replicates.

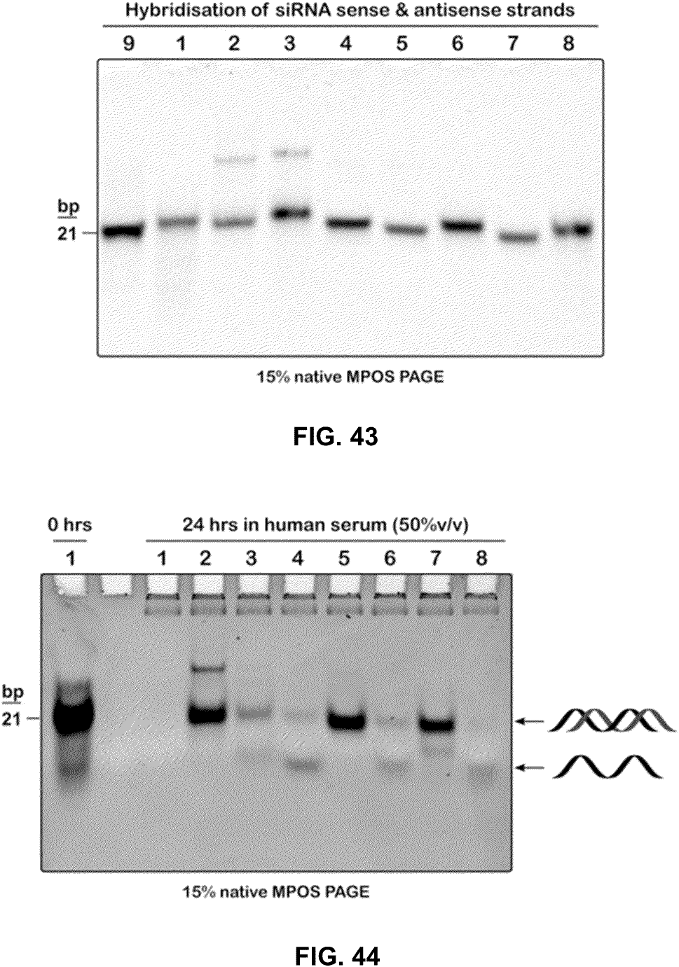

FIG. 43 shows the results of non-denaturing PAGE to verify sense and antisense strand hybridisation.

FIG. 44 shows the results of non-denaturing PAGE to demonstrate serum stability of siRNAs. siRNA duplexes were incubated for 24 h at 37° C. in 50% human serum.

### Example 5

siRNA molecules were attached to the core construct via disulphide bond formation. Thiol-containing RNA (S-1.1, C-1.1) at a final concentration of 50 µM was reduced in 50 mM TCEP for 1-2 hours. The reduced RNA was subjected to ethanol precipitation and the pellet air-dried. Thereupon, the siRNA sense strand pellet (S-1.1) was resuspended in a saturated solution of 2,2′-dithiodipyridine (ca. 100 mM) in 2.5 mM Tris pH 7.5, 50% v/v DMF, at a final RNA concentration of 100 µM. After 2 hours at room temperature, excess dithiodipyridine was removed by ethanol precipitation. The RNA pellet was then resuspended in coupling buffer (0.1 M phosphate buffer pH 7.7, 50% v/v formamide) to a final concentration of 100 µM. The solution was mixed with a 2-fold molar excess of core strand by transferring it into the tube containing the air-dried RNA pellet (C-1.1). The reaction was incubated at 40° C. for at least 1 hour, followed by HPLC purification. The purified products were loaded on denaturing PAGE (15%) in 1X TBE at a constant voltage of 250 V. Gel bands were visualized using GelRed™. 20 pmoles of RNA were loaded. Cr, unpurified crude reaction mixture; 1-6, different peaks in the HPLC trace.

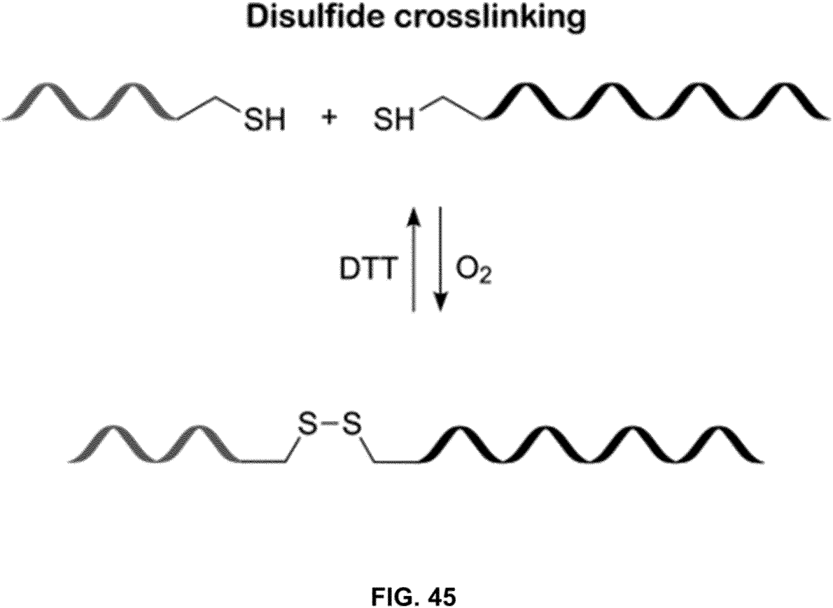

FIG. 45 is a schematic showing reversible thiol-disulfide exchange reaction used to functionalize nanoparticle core strands with siRNA.

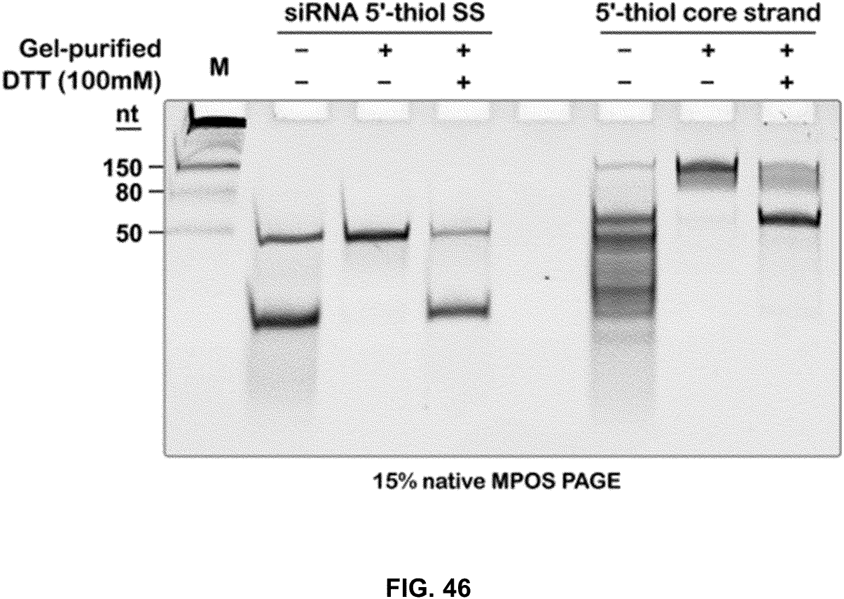

FIG. 46 shows the results of non-denaturing PAGE demonstrating the reversible self-dimerization of 5′ thiol-modified RNA strands. Under non-reducing conditions, thiol-bearing RNA strands couple with each other, resulting in a delay in electrophoretic mobility. After purification of the dimerised strands by gel extraction, a single confined band is observed. Incubation of the purified dimers in 100 mM DTT induces decoupling of the strands.

### Example 6

Copper azide-alkyne cycloaddition (CuAAC) was carried out according to two protocols; (1) To a 0.6 ml Eppendorf, previously flushed with N2, for a 20 µl reaction, were added azide containing RNA (3 µM), alkyne containing RNA (9 µM), PBS (10x, 2 ul), 0.6 µL of a 20% (v/v) acetonitrile/water solution. After degassing the reaction mixture, 0.3 µL of a degassed 10 mM sodium ascorbate solution (freshly prepared) was added, followed by the freshly prepared copper sulfate (0.1 µl, 5 mM stock solution). The reaction mixture was once again degassed and was incubated for 1 h at rt or in a heat block at 40° C. Then, an additional 0.1 µl of degassed sodium ascorbate solution was added to the reaction mixture. The reaction mixture was once again degassed and was incubated for 1 h at rt or in a heat block at 40° C. Clicked products were analyzed by denaturing PAGE. (2) To a 0.6 ml Eppendorf, previously flushed with N2, were added azide containing RNA (1 nmol, 1 µl), alkyne containing RNA (1.5 nmol, 1 µl), MgC12 (20 mM, 0.5 µl), TEAA (0.4 M, 1 µl, pH=7), DMSO (5 µl) and fresh ascorbic acid (25 mM, 1 µl). After degassing the reaction mixture, the freshly prepared CuBr-TBTA solution (50 mM CuBr / 50 mM TBTA 1:2 (v/v) in DMSO/t-BuOH 3:1 (v/v), 0.5 µl) was added. The reaction mixture was once again degassed and left on a shaker at rt for 1-2 hours. Clicked products were analyzed by denaturing PAGE. An RNA-RNA coupling efficiency of ~10-50% was observed.

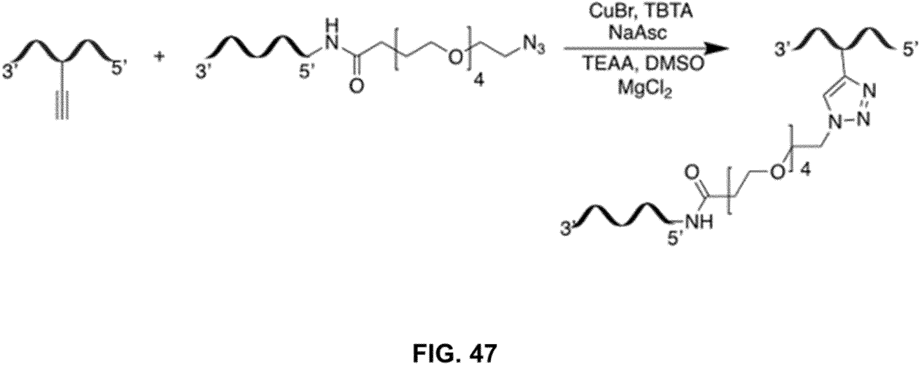

FIG. 47 is a schematic of a coupling reaction of an azide-functionalized oligo with a 2′O-modified alkyne moiety within an internal position on the second oligo use to insert CuAAC at an internal position on an RNA strand.

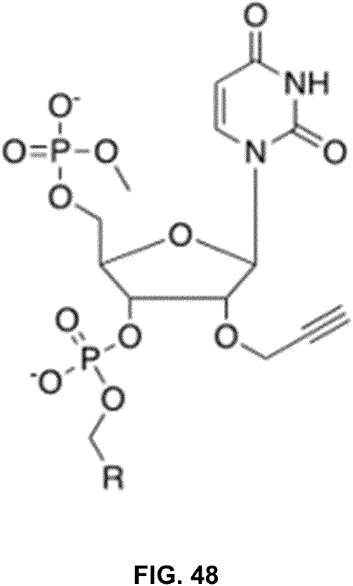

FIG. 48 shows the structure of a nucleotide bearing the alkyne group within the RNA strand.

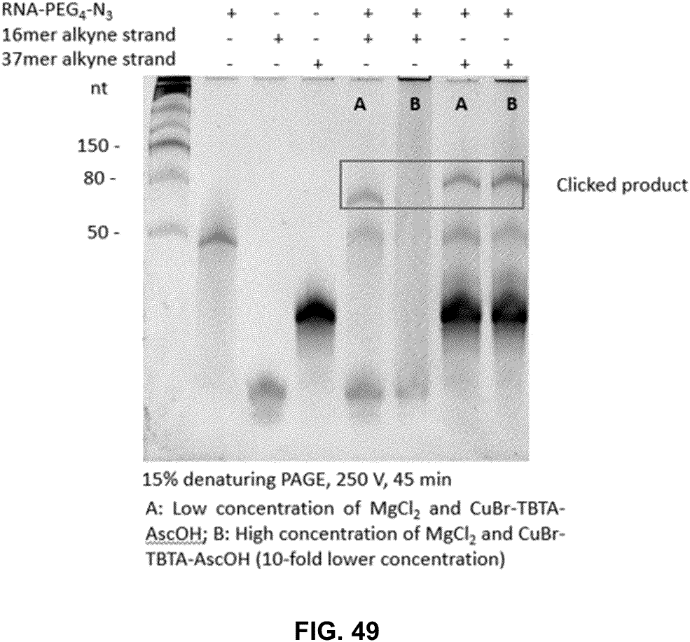

FIG. 49 shows the results a denaturing PAGE following coupling reactions.

### Example 7

Amine-functionalized RNA (100 µM) was coupled to a 10-fold excess (1 mM) of SM(PEG)6 in PBS (pH = 7) at rt for 30 min. Successful coupling was determined via denaturing PAGE and excess maleimide-linker was removed via RP HPLC. Then, 5′ thiol-functionalized RNA (100 µM) was reduced in the presence of TCEP and the coupling was carried out in PBS (pH = 7) with a 2 to 10-fold excess of one strand over another.

An RNA-peptide coupling efficiency of ~30% was observed for linear peptide, while it was ~10x lower with the branched peptide.

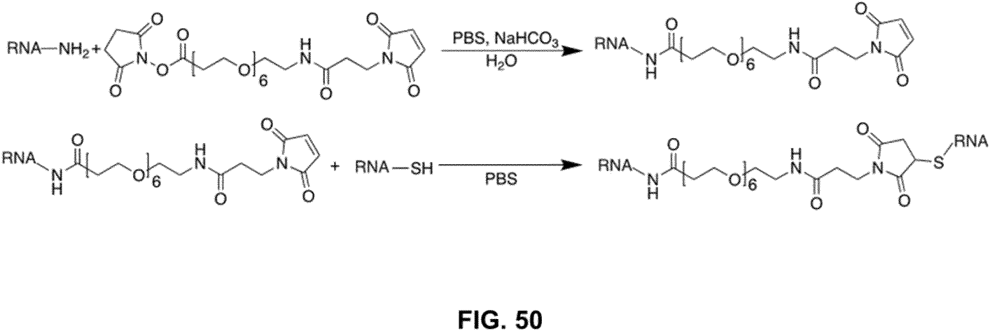

FIG. 50 is a schematic showing the coupling of an endosomal escape peptide to RNA via the NHS-PEG6-Maleimide linker. Top panel shows conjugation of peptide to NHS-PEG-Maleimide, and bottom panel shows conjugation of Maleimide containing peptide to TCEP-reduced thiol modified RNA.

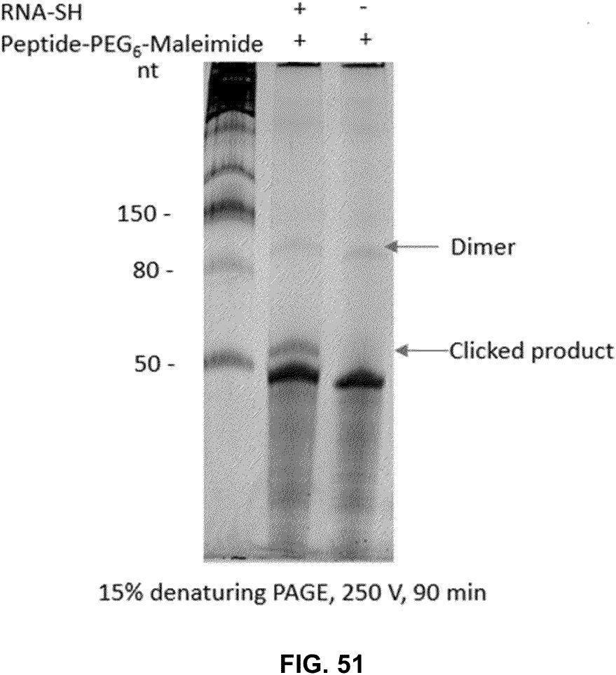

FIG. 51 shows the results of denaturing PAGE of a linear peptide conjugated to RNA.

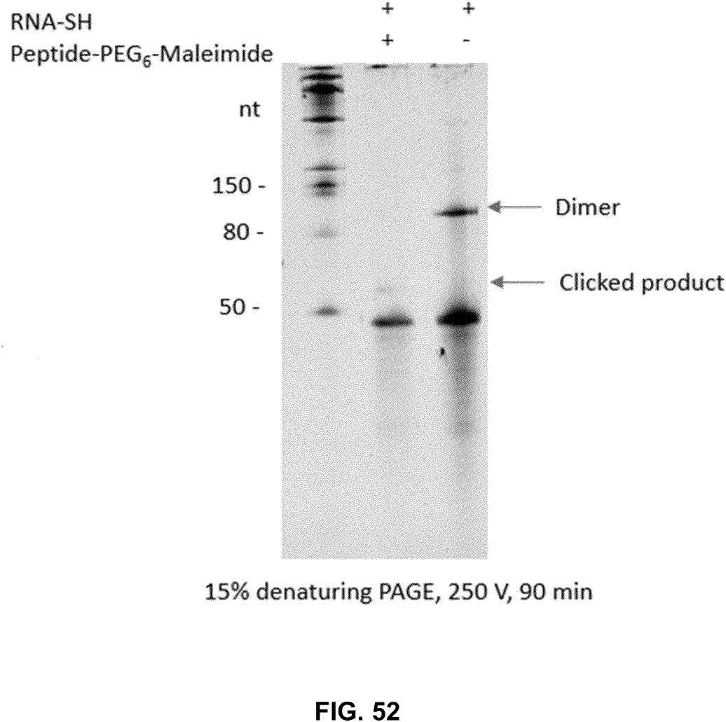

FIG. 52 shows the results of denaturing PAGE of a branched peptide conjugated to RNA.

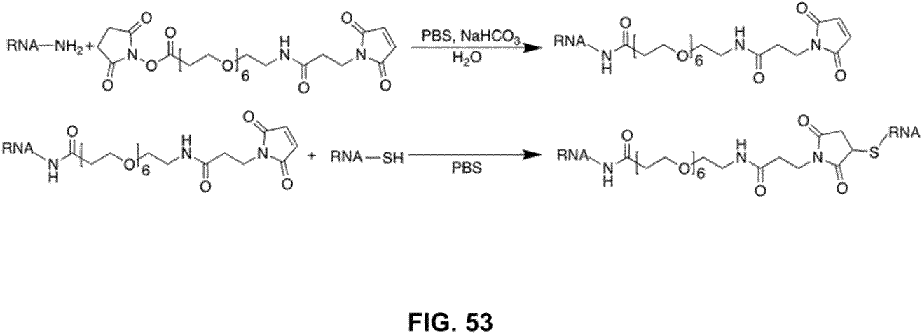

FIG. 53 is a schematic of disulphide linkage using NHS-PEG₆-Maleimide. Top panel shows coupling between an amine-containing RNA strand and NHS-PEG₆-Maleimide, and bottom panel shows coupling between maleimide-containing RNA and a thiol containing RNA strand.

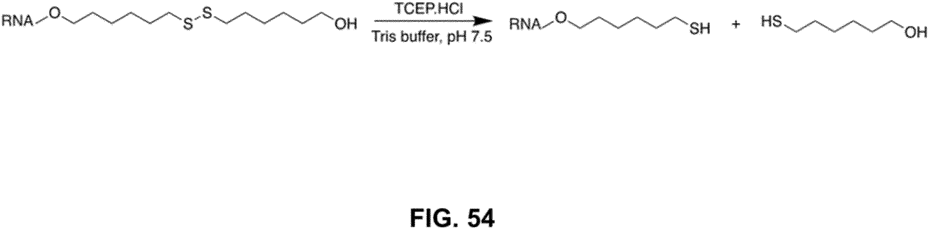

FIG. 54 is schematic of TCEP reduction of RNA modified with thiol-modifier C6 S-S at the 5′ position.

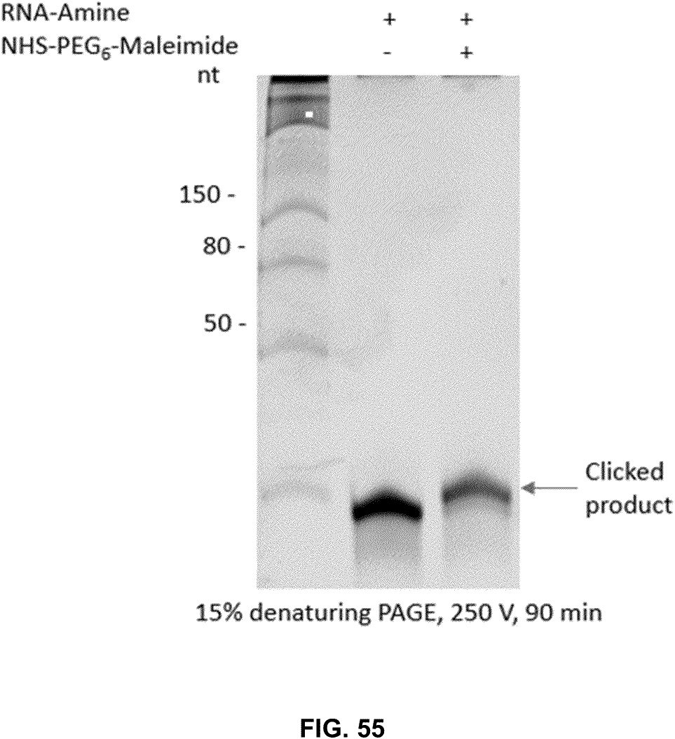

FIG. 55 shows results of denaturing PAGE following first coupling reaction.

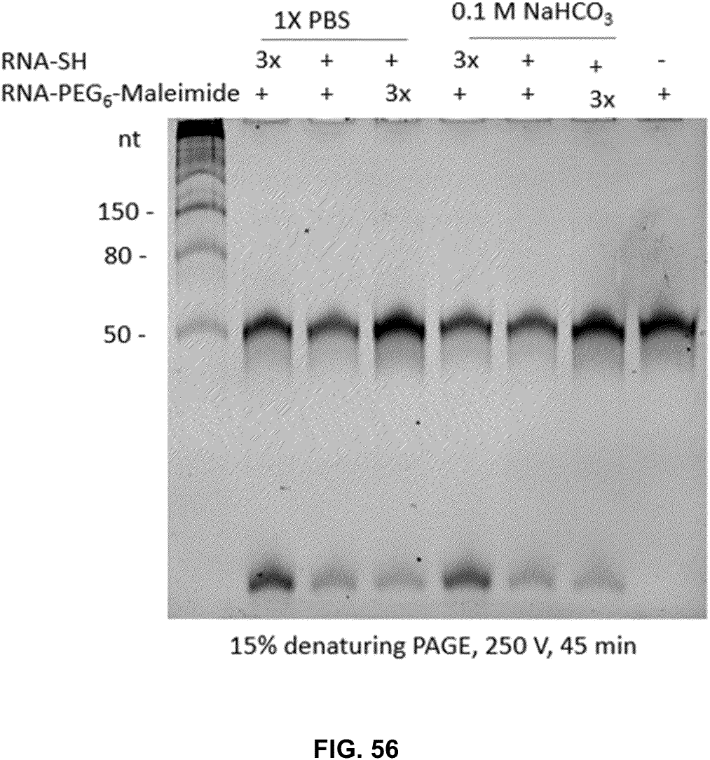

FIG. 56 shows results of denaturing PAGE following second coupling reaction.

### Example 8

Strain-promoted azide-alkyne cycloaddition (SPAAC) was carried out according to two protocols: (1) To a 0.6 ml Eppendorf, azide containing RNA (500 pmol, 1 µl) was mixed with DBCO containing RNA (2-fold excess 1000 pmol, 1 µl) in a solution of sodium chloride (0.25 M, 5.9 µl) containing EDTA (0.5 M, 0.1 µl) and HEPES (0.1 M, 2 µl). The reaction was incubated at rt for 2 hours. Clicked products were analyzed by denaturing PAGE. (2) To a 0.6 ml Eppendorf, azide containing RNA (1500 pmol, 1.5 µl) was mixed with DBCO containing RNA (500 pmol, 2.5 µl) in acetonitrile and TEAA/MeCN (0.1 M). The reaction (10 µl total volume) was incubated at rt for 2 hours. Clicked products were analyzed by denaturing PAGE.

An RNA-RNA coupling efficiency of ~20-70% was observed when using aqueous conditions, while in TEAA/MeCN conditions the coupling efficiency was ~10x lower.

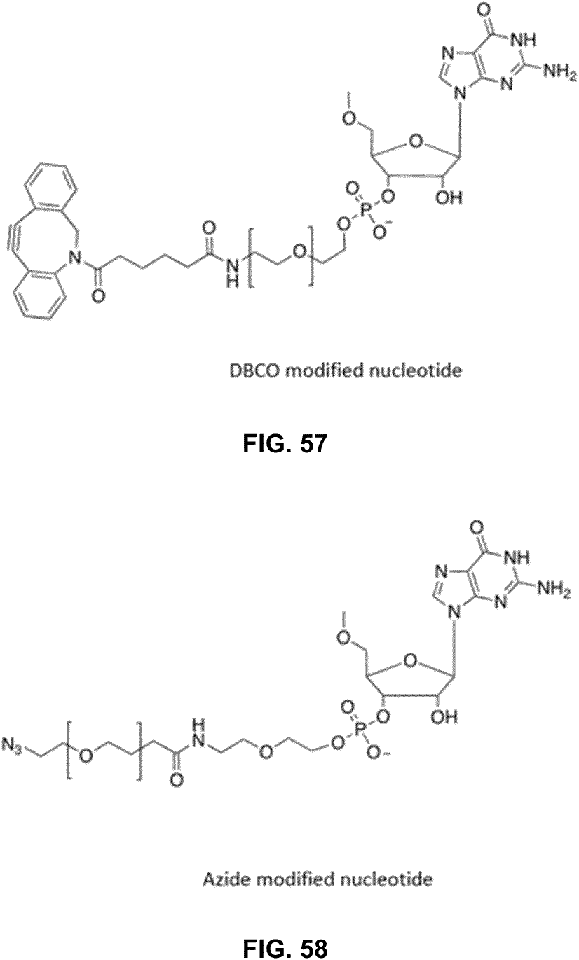

FIG. 57 shows the structure of a DBCO-modified nucleotide at the 5′ end of an RNA strand.

FIG. 58 shows the structure of an azide-modified nucleotide at the 5′ end of an RNA strand.

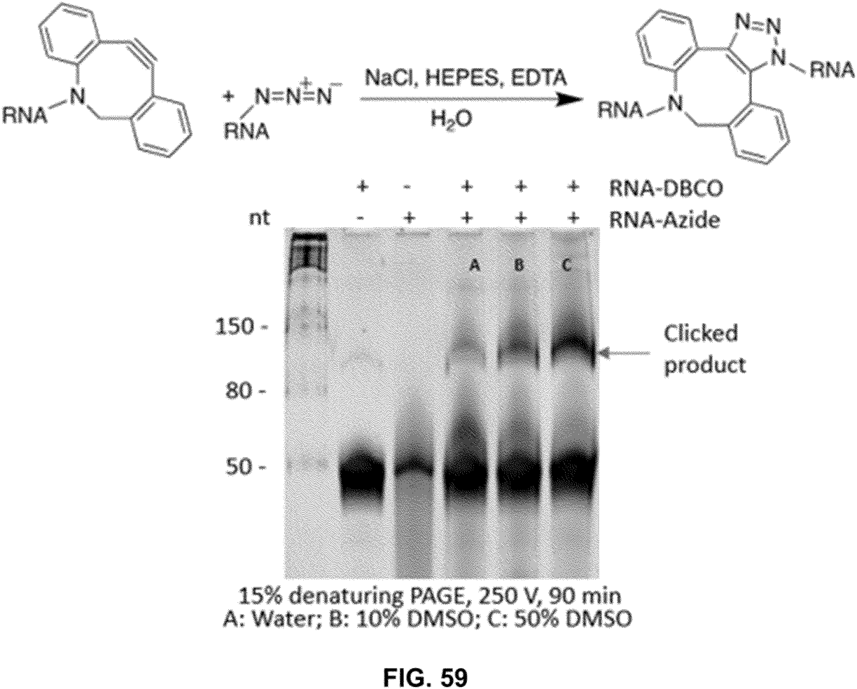

FIG. 59 shows the results of denaturing PAGE following the coupling reaction using aqueous conditions.

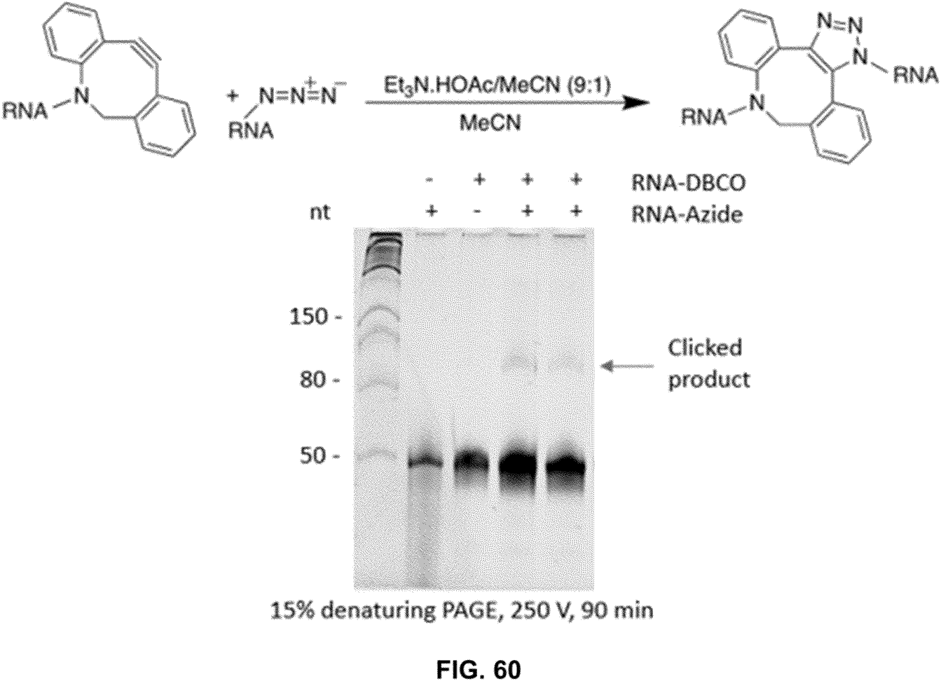

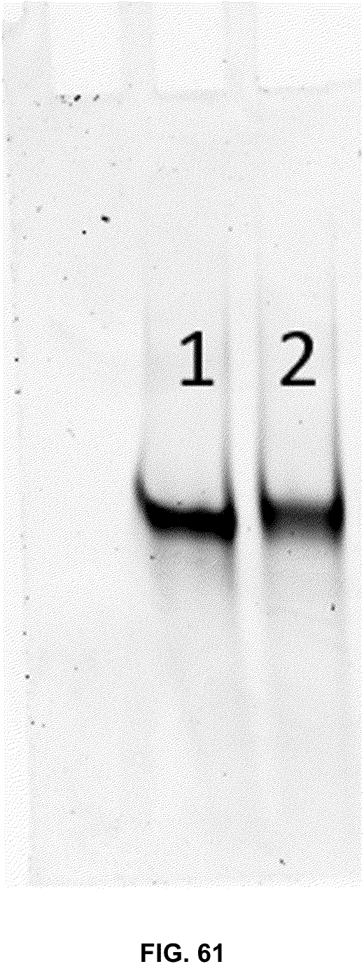

FIG. 60 shows the results of denaturing PAGE following the coupling reaction using TEAA/MeCN conditions.

### Example 9

A biotinTEG phosphoramidite was added to strand C-5.0 via the usual RNA synthesis protocol (see Example 1). SQ1 nanoparticles with and without a biotin-labelled strand were assembled following the protocol outlined in Example 2.

FIG. 61 shows the results of native 6% PAGE following biotin labelling. Protocol for assembly (one-pot) and for gel was indicated above; 1 = core construct (SQ1-0000-001); 2 = core construct with biotin (SQ1-0001-001). The results show that the addition of biotin did not disrupt assembly.

### Example 10

A number of different cell types have been trialled for a FACS based fluorescent molecule uptake assay. These include stably transfected HEK293T cells overexpressing the truncated mutant EGFRvIII, which is characterised by a deletion of 267 amino acids in the extracellular domain and results in a receptor unable to bind ligand yet constitutively active. WT cells with various endogenous levels of EGFR expression have also been trialled: HELA cells (EGFR+), MCF-7 (EGFR-), MDA-MB-231 (EGFR+), and A549 (EGFR+). Seeding densities used were identical for all experiments and described below.

Cells were plated with 1x10⁵ cells per well of 24 well plates. 24 hours later cells were treated with 200 nM of specified aptamer (A-1.0 or A-2.0) or nucleic acid nanoparticle in 250 µL DMEM media for 2 hours. To terminate internalisation, media was removed from cells and washed with 500 µL Dulbecco’s Phosphate Buffered Saline (D-PBS), followed by trypsinisation, neutralisation in DMEM media and transfer to labelled 1.5 mL Eppendorf tubes. After centrifugation at 200xg for 4 minutes to pellet cells, media was removed and cells resuspended in 500 µL D-PBS. Centrifugation was repeated, followed by resuspension in ice cold D-PBS. From this point all samples were kept on ice and centrifuged at 4° C. After a final centrifugation cells were resuspended in 200 µL MACS buffer (phosphate-buffered saline supplemented with 2 % (v/v) fetal calf serum and 1 mM EDTA) and immediately 10,000 events collected by flow cytometry (BD Biosciences FACSCanto; FacsDiva software). FloJo v10 software used to analyse data. Propidium iodide was used to stain live cells; 10 µL of 1 mg/mL per sample added immediately before analysis.

Results showed selected SXF1 aptamer (A-1.0) as the optimal targeting moiety as it displayed 10-fold higher EGFR targeting specificity when compared to a scramble control aptamer (A-3.0). SQ1-1000-001 construct functionalized with SXF1 aptamer (A-1.0) specifically internalises in EGFR+ cells 2-hours post-incubation (5-fold increase).

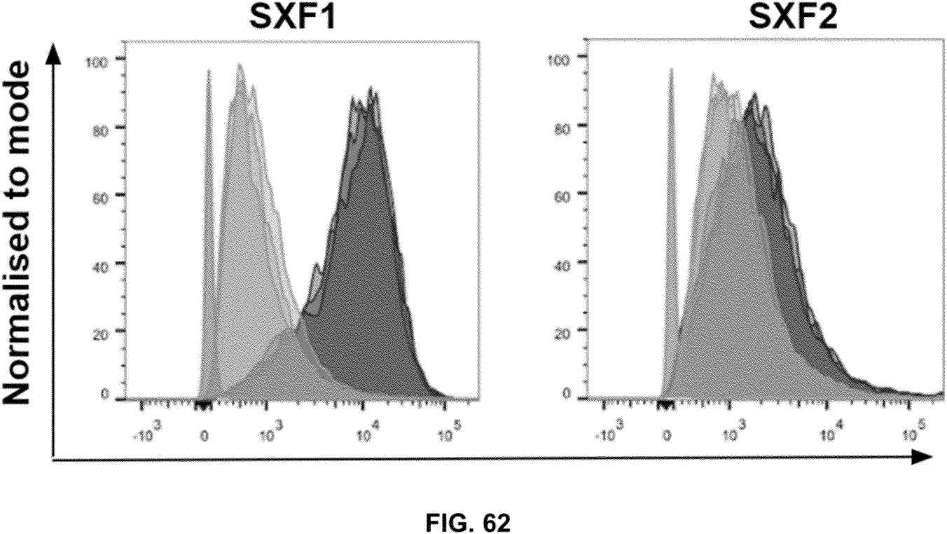

FIG. 62 shows graphs of uptake of functionalized SFX1 and SFX2 aptamers analysed by flow cytometry. SXF1 aptamer (E07min; A-1.0) is more specifically uptaken than SXF2 (CL4 aptamer) by EGFRvIII overexpressing cells. Representative flow cytometry histogram from one experiment, with 3 technical replicates overlaid, showing specific uptake of Cy5-labelled aptamer by a shift in fluorescence between control (green) and EGFRvIII overexpressing (purple) HEK-293T cells, for the corresponding aptamers. The aptamers used herein are known in the literature and described in [E07min, A-1.0] N. Li, H.H. Nguyen, M. Byrom, A.D. Ellington, Inhibition of Cell Proliferation by An Anti-EGFR Aptamer, PLoS One. 6 (2011) 1-10. https://doi.org/10.1371/jounal.pone.0020299; [SXF2, CL4, A-2.0] C.L. Esposito, D. Passaro, I. Longobardo, G. Condorelli, P. Marotta, A. Affuso, V. de Franciscis, L. Cerchia, A Neutralizing RNA Aptamer against EGFR Causes Selective Apoptotic Cell Death, PLoS One. 6 (2011) e24071. https://doi.org/10.1371/journal.pone.0024071, the contents of which are incorporated herein by reference.

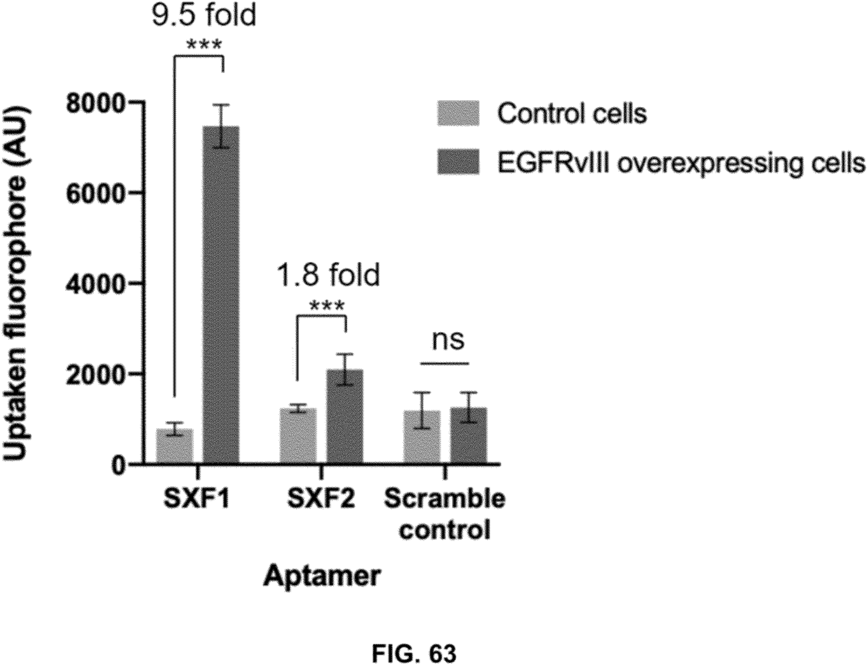

FIG. 63 is graph showing uptake of nucleic acid nanoparticles by EGFR expressing cells. SXF1 aptamer (E07min, A-1.0) is specifically uptaken by EGFR expressing cells.

Geometric mean quantification of flow cytometry results after normalisation by removal of respective background fluorescence in untreated cells. Numbers above the bars are calculated fold increase in internalisation between control and EGFRvIII overexpressing HEK293T cells. The sequence of the scramble control aptamer (A-3.0) is literature-known, and is incorporated herein by reference: [1] C.L. Esposito, D. Passaro, I. Longobardo, G. Condorelli, P. Marotta, A. Affuso, V. de Franciscis, L. Cerchia, A neutralizing RNA aptamer against EGFR causes selective apoptotic cell death, PLoS One. (2011). https://doi.org/10.1371/journal.pone.0024071.

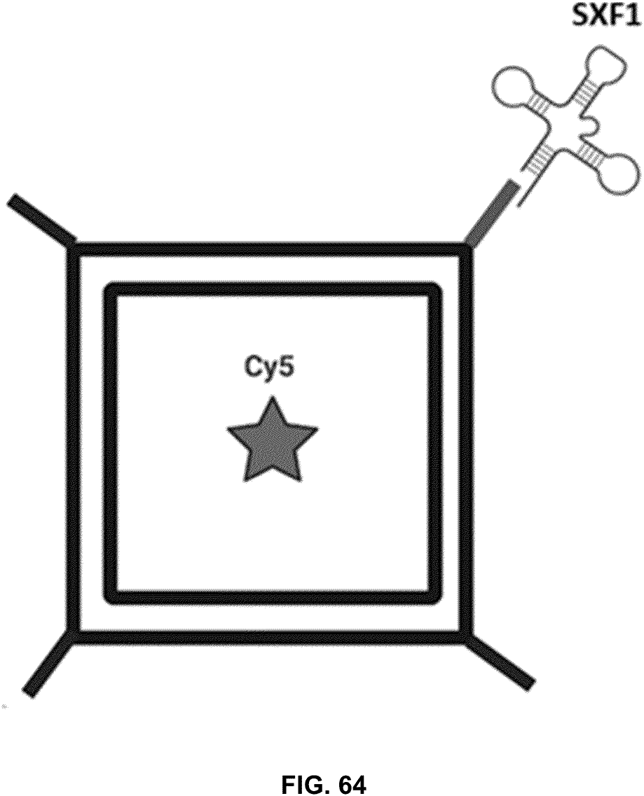

FIG. 64 is a schematic of a Cy5-labelled construct with the most optimal SXF1 aptamer annealed through complementary base-pairing (SQ1-1000-001).

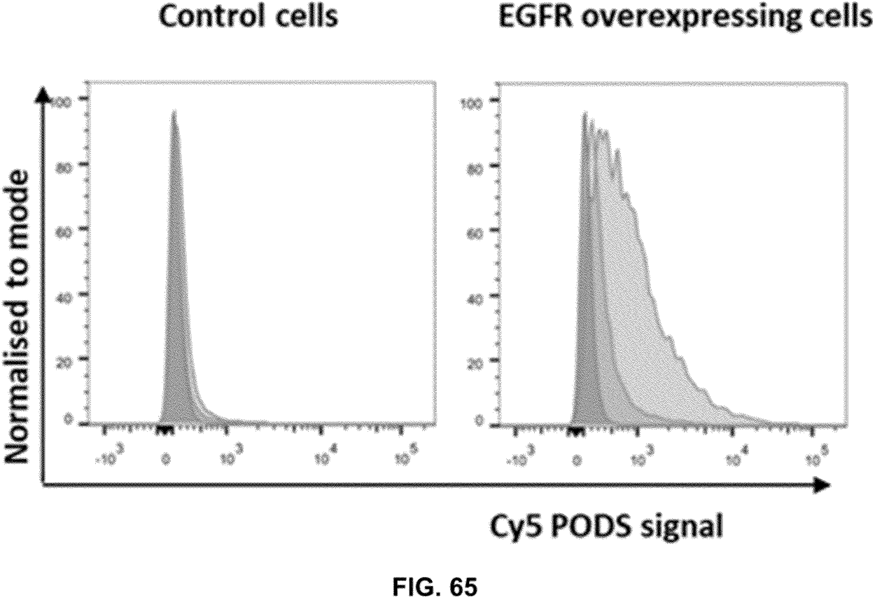

FIG. 65 shows FACS plots showing the enhancement of Cy5-labelled nanoparticle uptake by the SXF1 aptamer, specifically in cells overexpressing EGFRvIII.

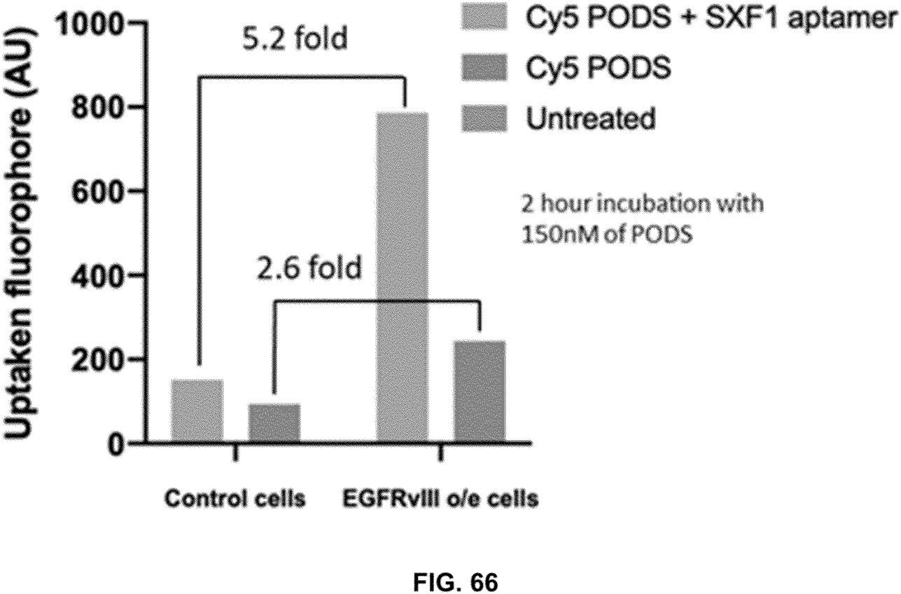

FIG. 66 is graph of fluorescence uptake showing the enhancement of Cy5-labelled nanoparticle uptake by the SXF1 aptamer, specifically in cells overexpressing EGFRvIII.

### Example 11

Cytotoxicity and efficiency of mRNA silencing using siRNA was analysed. Cancer cells were transfected with 0 to 20 nM siRNA directed against PLK1, a serine/threonine kinase involved in the regulation of cell cycle progression. Transfections of siRNA attached to an RNA nanoparticle were compared to transfections of free siRNA.

For cytotoxicity experiments, transfections were performed in white 96-well plates at a seeding density of 1,000 cells/well and cell viability was assayed five days after transfection using Promega’s CellTiter-Glo assay according to manufacturer’s instructions. As a positive control for cell death, cells were treated with 0.1% (v/v) triton x-100 (Sigma Aldrich) in serum-free DMEM, followed by incubation at 37° C. for 30 min. Luminescence was recorded on a Tecan M200 plate reader.

For quantitative PCR, transfections were performed in 24-well plates at a seeding density of 50,000 cells/well and total RNA was isolated 48-72 hours after transfection using the Monarch Total RNA Miniprep Kit (New England Biolabs). cDNA was reverse-transcribed from 500 ng RNA mixed with 0.5 µL random primers, 1 µL PCR nucleotide mix, 4 µL 5 X First Strand, 1 µL 0.1 M DTT, 1 µL RNAse out and 0.5 µL superscript III reverse transcriptase under the following conditions: 25° C. for 5 min, 50° C. for 1 h and 70° C. for 15 min. After cooling to room temperature, 5 µL of 1:30 diluted cDNA was mixed with 7.5 µL PowerUp SYBR Green Master Mix (Thermo Fisher Scientific), 1 µL of 5 µM combined forward and reverse primers and 1.5 µL RNAse free water. The 15 µL reaction mixture was loaded per well for each gene of interest and plated at least in duplicate. The qPCR cycle was run on QuantStudio 5 for 2 min at 50° C. (1 cycle); 2 min at 95° C., 1 s at 95° C. and 30 s at 60° C. (40 cycles); followed by melt curve analysis (1 s at 95° C., 30 s at 60° C., and a ramp-up from 60° C. to 90° C. with continuous fluorescence measurements).

For Western Blot analysis, transfections were performed in 24-well plates at a seeding density of 50,000 cells/well and cells were lysed in 100 µL radioimmunoprecipitation assay (RIPA) buffer containing 150 nM NaCl, 1% Triton X-100, 0.5% sodium deoxycholate, 0.1% sodium dodecyl sulfate, 50 mM Tris pH 8 supplemented with protease inhibitors cocktail V (Roche) on ice. After centrifugation at 20,000 rcf at 4° C. for 10 min to remove insoluble material, the concentration of protein in the supernatant was measured using the Pierce BCA protein assay kit according to manufacturer’s instructions. Cell lysates containing 20-30 µg protein were mixed with 4x Laemmli protein sample buffer (Bio-Rad) supplemented with 10% (v/v) 2-mercaptoethanol (Sigma) and heated to 95° C. for 10 min prior to loading. Protein samples were electrophoresed on pre-cast 4-20% MiniPROTEAN TGX stain-free gels in 25 mM Tris, 190 mM Glycine, 0.1% SDS buffer at 100 V until the dye front reached the bottom. Proteins were blotted onto 0.2 µm polyvinylidene difluoride mini membranes using a turbo transfer system (Bio-Rad) at 1.3 A 25 V for 7 min. After blocking with 5 (w/v) milk powder (Marvel) in tris-buffered saline with Tween (TBS-T) (25 mM Tris, 140 mM sodium chloride, 3 mM potassium chloride and 0.1% (v/v) tween-20 (Fisher Bioreagents), membranes were probed for primary antibodies overnight at 4 C in 1% (w/v) milk TBS-T followed by three washes in TBS-T and incubation with corresponding secondary peroxidase antibodies in 1% (w/v) milk TBS-T for 1 h. Protein bands were visualised using CCD chemiluminescence by applying 1 mL ECL substrate (Bio-Rad) to the membrane (Azure biosystems c600).

PLK1-specific knock-down leads to cytotoxic effects in the breast (MDA-MB-231), lung (A549) and cervical (Hela) cancer cell line models presented. A concentration of 0.5 nM is sufficient to detect gene downregulation and >50% cytotoxicity and 10 nM leads to >90% cell death. Thiol-based coupling to the nucleic acid nanostructure preserves siRNA silencing activities

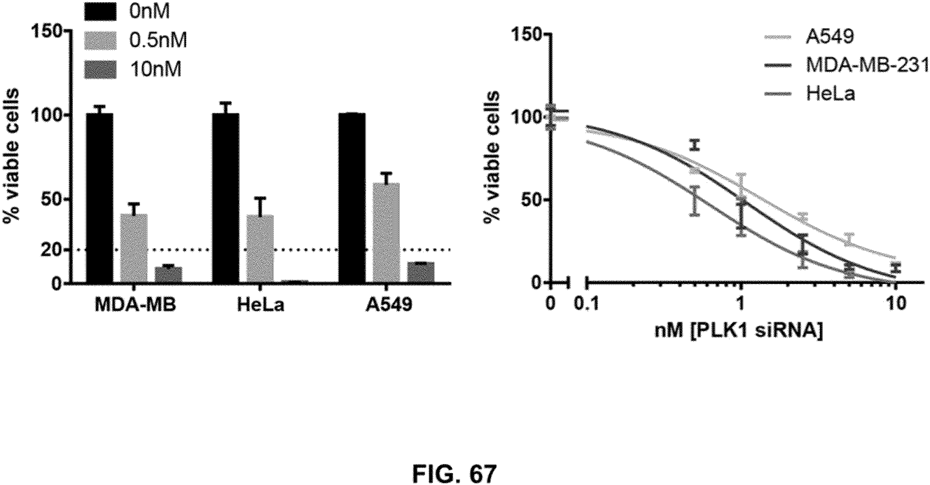

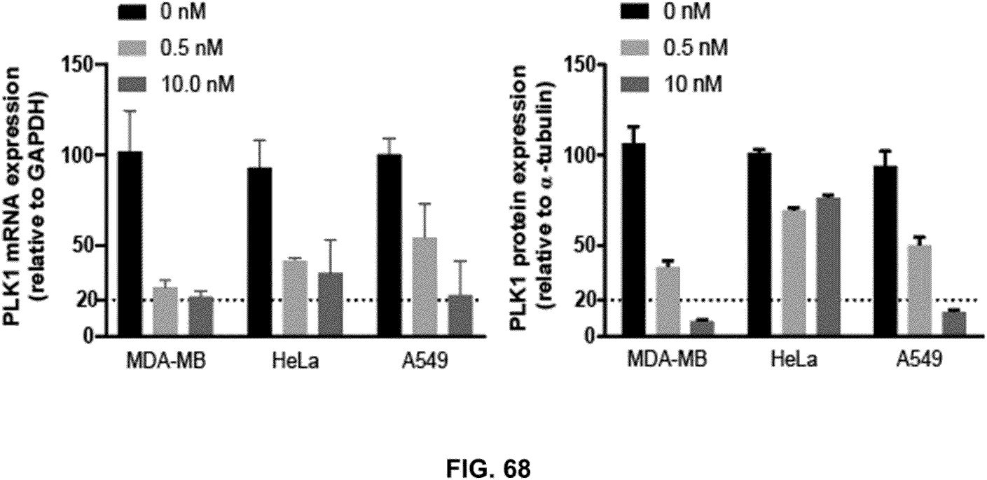

FIG. 67 shows graphs of cell viability following uptake of PLK1-targeting siRNA (AMB) in cancer cells. The graph on the left shows the dose-dependent cytotoxicity of PLK1-targeting siRNA in cancer cells, and the graph on the right shows cytotoxic activity of free PLK1 siRNA 5 days after transfection, at siRNA doses ranging from 0 nM to 10 nM. Data were normalized to triton-treated cells (0% viable cells) and untransfected cells (100% viable cells).

FIG. 68 shows graphs of PLK1 mRNA and protein expression following uptake of PLK1-targeting siRNA (AMB). The graph on the left shows the effect of PLK1 silencing at the RNA level as measured by qPCR, normalised to the expression level of GAPDH and relative to cells transfected with 10 nM non-targeting siRNA. The graph on the right shows show the effect on PLK1 protein expression as quantified by Western Blotting. Western Blot data were normalized to α-tubulin expression and are shown relative to cells transfected with 10 nM non-targeting siRNA (ctrl). Gene silencing efficacy of free PLK1 siRNA (AMB) was measured 73 hours after transfection, at siRNA doses ranging from 0 nM to 10 nM.

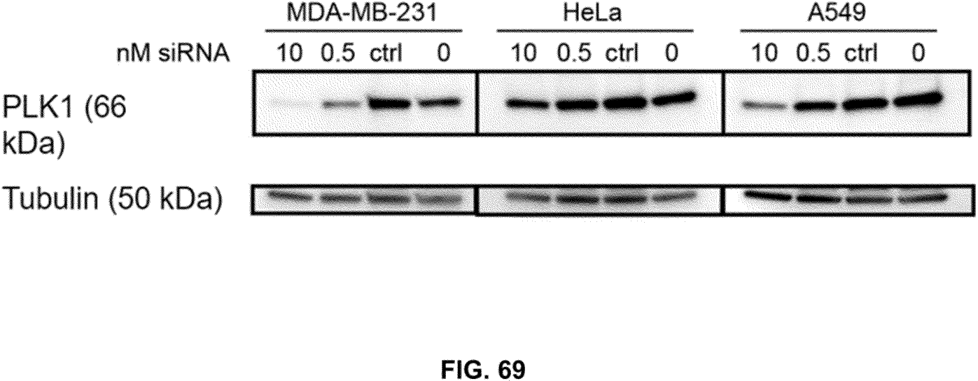

FIG. 69 shows results of western blotting for PLK1 following uptake of PLK1-targeting siRNA.

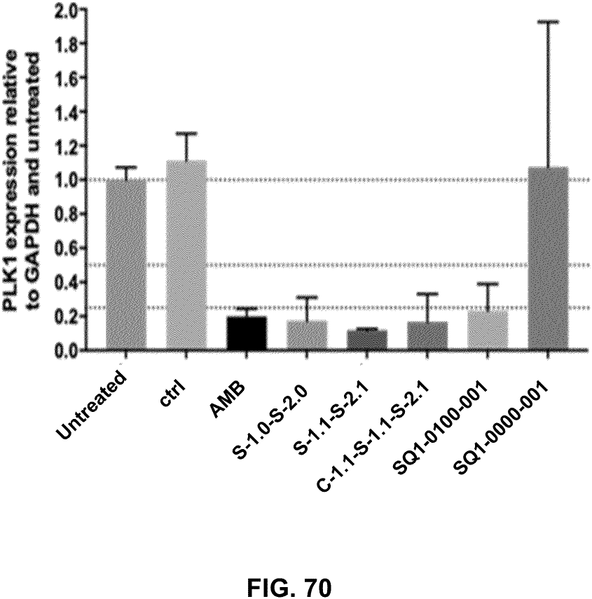

FIG. 70 is a graph PLK1 mRNA expression following treatment with siRNA attached to an RNA scaffold. The gene silencing efficacy of free PLK1 siRNA (10 nM) (AMB, S-1.0-S-2.0, S-1.1-S-2.1) in comparison to thiol-coupled siRNA attached to an RNA core strand (10 nM) (C-1.1-S-1.1-S-2.1) or an RNA nanoparticle (10 nM) (SQ1-0100-001), as assessed by qPCR. Experiment was performed in MDA-MB-231 breast cancer cells. Data were normalised to the expression level of GAPDH and are shown relative to cells transfected with 10 nM non-targeting siRNA (ctrl). The sequences used in the experiment are provided in Table 7.

TABLE 7

| | | |
|---|---|---|
| Sample | siRNA sequence (5′-3′) | Modifications |
| Untreated | N/A | |
| ctrl | undisclosed (AllStars Negative Control siRNA, Qiagen) | |
| AMB | SS: GCAAUUACAUGAGCGAGCATT AS: UGCUCGCUCAUGUAAUUGCGG | undisclosed |
| S-1.0-S-2.0 | S-1.0: GCAAUUACAUGAGCGAGCATT S-2.0: UGCUCGCUCAUGUAAUUGCGG | N/A |
| S-1.1-S-2.1 | S-1.1: GcAAuuAcAuGAGcGAGcATT S-2.1: uGcucGcucAuGuAAuuGcGG | 2′F C, U (both strands) 5′ thiol (SS) |
| C-1.1-S-1.1-S-2.1 | C-1.1: GGGAAAcucuGucGuGGGAcGGucAGAcuGuucAAccAcuccucuuc S-1.1: GcAAuuAcAuGAGcGAGcATT S-2.1: uGcucGcucAuGuAAuuGcGG | 2′F C, U (all strands) 5′ thiol (C-1.1, S-1.1) |
| SQ1-0100-001 | assembled from strands C-1.1 coupled to siRNA S-1.1-S-2.1, C-2.0, C-3.0, C-4.0, C-5.0 | 2′F C, U (all strands) 5′ thiol (C-1.1, S-1.1) |
| SQ 1-0000-001 | assembled from strands C-1.1, C-2.0, C-3.0, C-4.0, C-5.0 | 2′F C, U (all strands) 5′ thiol (C-1.1) |

### Example 12

A series of positively charged and hydrophobic nucleosides were synthesised as mediators of endosomal escape.

1-((6aR, 8R, 9R, 9aS)-9-hydroxy-2, 2, 4, 4-tetraisopropyltetrahydro-6H-furo[3, 2-ƒ][1,3,5,2,4]trioxadisilocin-8-yl)pyrimidine-2, 4(¹H,3H)-dione

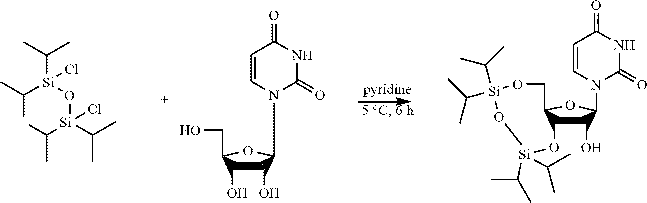

Modified from a procedure outlined by Li and co-workers (E. Li, Y. Wang, W. Yu, Z. Lv, Y. Peng, B. Liu, S. Li, W. Ho, Q. Wang, H. Li, J. Chang, Synthesis and biological evaluation of a novel β-D-2′-deoxy-2′-α-fluoro-2′-β-C-(fluoromethyl)uridine phosphoramidate prodrug for the treatment of hepatitis C virus infection, Eur. J. Med. Chem. 143 (2018) 107-113. https://doi.org/10.1016/j.ejmech.2017.11.024.).

Uridine (5 g, 20.47 mmol) was dissolved in pyridine (50 mL). 8.2 mL (25.6 mmol) of 1,3-dichloro-1,1,3,3-tetraisopropyldisiloxane (TIPDSCI) was slowly added at 5° C. The resulting solution was stirred at 5° C. for 6 h. Then, 30 mL of HCl 1 M was added. The aqueous layer was re-extracted with DCM (3 x 10 mL). The combined organic layers were dried with Na2SO4, filtered and the solvent evaporated. The crude residue was purified by flash chromatography (RP-C18, H2O:MeCN 10-65%). The title compound was afforded as a white solid (7.03 g, 71%).

1HNMR (CDC13, 400 MHz) δ (ppm): 7.67 (d, J = 8.2 Hz, 1H), 5.72 (d, J = 0.9 Hz, 1H), 5.69 (dd, J = 8.1, 1.7 Hz, 1H), 4.39 (dd, J = 8.6, 4.9 Hz, 1H), 4.25 - 4.14 (m, 2H), 4.09 (dt, J = 8.7, 2.6 Hz, 1H), 4.05 - 3.93 (m, 1H), 3.49 (s, 6H), 1.22 - 1.06 (m, 9H), 1.06 - 0.83 (m, 10H). 13C NMR (CDC13, 100.6 MHz) δ (ppm): 163.3, 150.1, 140.0, 102.0, 91.0, 82.0, 75.3, 69.0, 60.3, 50.9, 17.5, 17.4, 17.4, 17.3, 17.1, 17.0, 17.0, 16.9, 13.5, 13.0, 12.6. ESI-HRMS(+) m/z (%): calc. C₂₁H₃₉N₂O₇Si₂ 487.2296; exp.487.2295 [M+H]+.

Methyl(((6aR,8R,9R,9aR)-8-(2,4-dioxo-3,4-dihydropyrimidin-1(2H)-yl)-2,2,4,4-tetraisopropyltetrahydro-6H-furo[3,2-∱[1,3,5,2,4]trioxadisilocin-9-yl)oxy)acetate

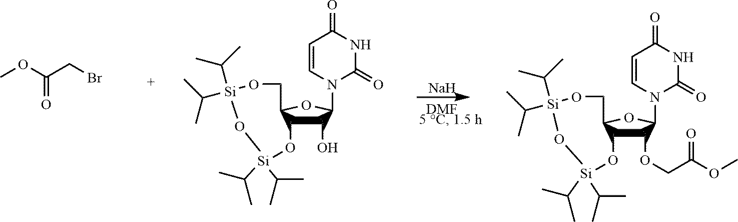

Modified from a procedure outlined by Milton and co-workers (S. Milton, C. Ander, D. Honcharenko, M. Honcharenko, E. Yeheskiely, R. Strömberg, Synthesis and stability of a 2′-O-[N-(aminoethyl)carbamoyl] methyladenosine-containing dinucleotide, European J. Org. Chem. (2013) 7184-7192. https://doi.org/10.1002/ejoc.201300699.).

Sodium hydride (60%) (28 mg, 0.709 mmol) was added to a solution of 1-((6aR,8R,9R,9aS)-9-hydroxy-2,2,4,4-tetraisopropyltetrahydro-6H-furo[3,2-f][1,3,5,2,4]trioxadisilocin-8-yl)pyrimidine-2,4(1H,3H)-dione (300 mg, 0.616 mmol) in anhydrous DMF (30 mL) at rt. After stirring for 1 h, methyl 2-bromoacetate (67 µL, 0.709 mmol) was added. The resulting solution was then stirred for 16 h . Then, water was added and the solution was extracted with EtOAc (3 x 20 mL). The combined organic layers were dried over Na2SO4, filtered, and concentrated in vacuo. The product was purified by flash chromatography (silica, PE:EtOAc 0-20% 4 CV, 20% 9 CV, PE:EtOAc 20-100% 15 CV). The title compound was obtained as a yellow oil (261 mg, 76%).

1HNMR (CDC13, 400 MHz) δ (ppm): 7.74 (d, J = 8.2 Hz, 1H), 5.83 - 5.74 (m, 2H), 4.70 (s, 2H), 4.37 (dd, J = 8.8, 4.9 Hz, 1H), 4.23 (dd, J = 13.3, 2.0 Hz, 1H), 4.15 (d, J = 4.9 Hz, 1H), 4.12 (dt, J = 8.8, 2.3 Hz, 1H), 4.02 (dd, J = 13.3, 2.8 Hz, 1H), 3.77 (s, 3H), 1.17 - 0.90 (m, 30H). 13C NMR (CDC13, 100.6 MHz) δ (ppm): 168.0, 162.0, 150.3, 138.2, 101.2, 91.1, 81.9, 75.4, 68.9, 60.2, 52.4, 41.5, 17.4, 17.4, 17.3, 17.2, 17.0, 16.9, 16.9, 16.8, 13.4, 12.9, 12.5.

3-Benzoyl-1-((6aR,8R,9R,9aS)-9-hydroxy-2,2, 4, 4-tetraisopropyltetrahydro-6H-furo[3,2-∱][1,3,5,2,4]trioxadisilocin-8-yl)pyrimidine-2, 4(1 H,3H)-dione

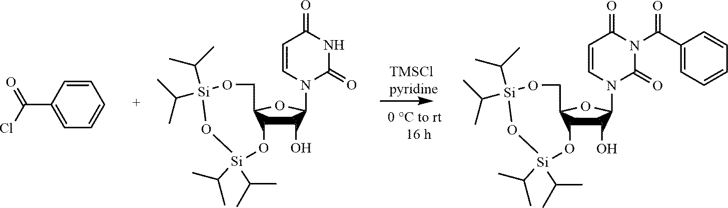

Modified from a procedure outlined by Zhu and co-workers (X.F. Zhu, H.J. Williams, A.I. Scott, An improved transient method for the synthesis of N-benzoylated nucleosides, Synth. Commun. 33 (2003) 1233-1243. https://doi.org/10.1081/SCC-120017200.).

TMSC1 (0.21 mL, 1.644 mmol) was added to a solution of 1-((6aR,8RXR,9aS)-9-hydroxy-2,2,4,4-tetraisopropyltetrahydro-6H-furo[3,2-f][1,3,5,2,4]trioxadisilocin-8-yl)pyrimidine-2,4(1H,3H)-dione (500 mg, 1.027 mmol) in 20 mL of anhydrous pyridine at rt. After stirring for 2 h, the mixture was cooled to 0° C. and 0.143 mL (1.233 mmol) of benzyl chloride was added dropwise. The solution was stirred at room temperature for 16 h. Then, water was added (20 mL) and stirred for 1 h. The mixture was concentrated in vacuo. The product was purified by flash chromatography (C18-RP, H2O:MeCN, 5-100% 45 CV) to afford 232.3 mg of the title compound (38%).

1HNMR (CDC13, 400 MHz) δ (ppm): 9.57 (s, 1H), 8.14 - 8.00 (m, 2H), 7.76 (d, J =8.2 Hz, 1H), 7.64 - 7.52 (m, 1H), 7.46 (t, J =7.8 Hz, 2H), 5.99 (s, 1H), 5.76 (d, J =8.1 Hz, 1H), 5.66 (d, J =5.0 Hz, 1H), 4.52 (dd, J = 9.1, 5.0 Hz, 1H), 4.28 (dd, J = 13.3, 1.7 Hz, 1H), 4.16 (dt, J = 9.1, 2.1 Hz, 1H), 4.04 (dd, J = 13.4, 2.7 Hz, 1H), 1.08 (ddd, J = 24.7, 6.4, 2.5 Hz, 25H), 0.88 (dd, J = 14.9, 3.8 Hz, 8H). 13C NMR (CDC13, 100.6 MHz) δ (ppm): 164.8, 163.5, 149.8, 139.5, 133.2, 129.8, 129.5, 128.4, 102.3, 88.7, 82.4, 75.9, 68.0, 59.7, 17.5, 17.4, 17.3, 17.2, 16.8, 16.8, 16.7, 13.4, 12.9, 12.5.

N-butyl(((6aR,8R,9R,9aR)-8-(2,4-dioxo-3,4-dihydropyrimidin-1(2H)-yl)-2,2,4,4-tetraisopropyltetrahydro-6H-furo[3,2-∱][1,3,5,2,4]trioxadisilocin-9-yl)oxy acetamide

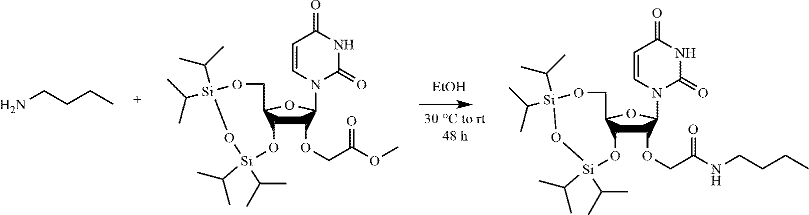

1-((6aR,8R,9R,9aS)-9-hydroxy-2,2,4,4-tetraisopropyltetrahydro-6H-furo[3,2-f][1,3,5,2,4]trioxadisilocin-8-yl)pyrimidine-2,4(1H,3H)-dione (100 mg, 0.179 mmol) was dissolved in in 2 mL of EtOH and butylamine (0.530 mL, 5.37 mmol) was added. The reaction mixture was stirred at 30° C. for 6 h, and then at rt for 2 days. The solvent was evaporated and the residue was dissolved in H2O, followed by extraction with EtOAc (3 x 10 mL). The organic layers were then combined, dried (Na2SO4), filtered, and evaporated. The residue was then purified by flash chromatography (silica, PE 4 CV, PE:EtOAc 0-20% 5 CV, 20% EtOAc y, PE:EtOAc 20-100% y), affording the title compound (54.9 mg, 51%).

1HNMR (CDC13, 400 MHz) δ (ppm): 7.75 (dd, J = 8.2, 1.6 Hz, 1H), 6.09 (t, J = 5.7 Hz, 1H), 5.77 (t, J = 4.1 Hz, 2H), 4.68 (d, J = 7.0 Hz, 1H), 4.62 - 4.49 (m, 1H), 4.34 (td, J = 9.1, 4.8 Hz, 1H), 4.22 (dd, J = 13.4, 1.7 Hz, 1H), 4.18 - 4.05 (m, 2H), 4.01 (dt, J = 13.3, 2.6 Hz, 1H), 3.76 (s, 1H), 3.33 - 3.18 (m, 2H), 1.57 - 1.36 (m, 2H), 1.36 - 1.17 (m, 2H), 1.17 - 0.95 (m, 30H), 0.91 (t, J = 7.3 Hz, 3H). 13C NMR (CDC13, 100.6 MHz) δ (ppm): 166.4, 162.5, 150.5, 138.0, 101.2, 91.1, 81.8, 75.3, 75.3, 68.8, 60.0, 52.5, 43.4, 41.6, 39.5, 31.5, 20.0, 17.4, 17.4, 17.3, 17.2, 17.0, 16.9, 16.9, 16.8, 13.7, 13.4, 13.4, 12.9, 12.5.

1-((6aR, 8R,9R,9aR)-2,2,4,4-tetraisopropyl-9-(2-(2-methoxyethoxy)ethoxy)tetrahydro-6H-∱uro[3,2-∱][1,3,5,2,4]trioxadisilocin-8-yl)pyrimidine-2,4(1H,3H)-dione

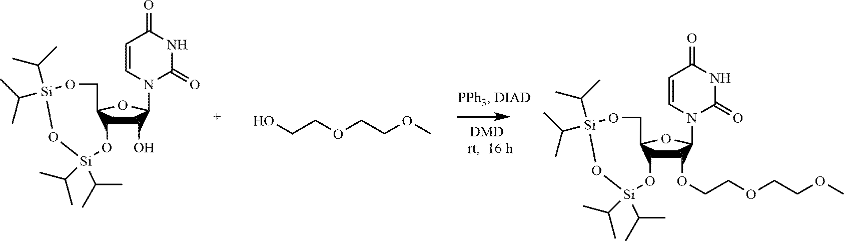

1-((6aR,8R,9R,9aS)-9-hydroxy-2,2,4,4-tetraisopropyltetrahydro-6H-furo[3,2-f][1,3,5,2,4]trioxadisilocin-8-yl)pyrimidine-2,4(1H,3H)-dione (0.2 g, 0.411 mmol) was dissolved in anhydrous DMF (3 mL). Add PPh3 (162 mg, 0.616 mmol) in 2 mL anhydrous DMF was added, as well as 58 µL (0.493 mmol) of 2-(2-methoxyethoxy)ethan-1-ol, and 121 µL (0.616 mmol) of DIAD in this order. The reaction mixture was stirred at rt for 16 h. H2O (15 mL) was then added (a white precipitate formed). The reaction mixture was then extracted with EtOAc (3 x 10 mL). The organic layers were combined, washed with brine (10 mL), dried (Na2SO4), filtered, and concentrated in vacuo. The compound was purified by flash chromatography (PE:EtOAc 0-100% 14 CV) and then re-purified by RP flash chromatography (C18-RP, H2O:MeCN 5-50% 24 CV, 50% 2 CV, 50-80% MeCN 14 CV, 80% 44 CV, 80-100% 10 CV).

1HNMR (CDC13, 400 MHz) δ (ppm): 7.65 (d, J = 8.1 Hz, 1H), 5.81 - 5.64 (m, 2H), 4.36 (dd, J = 8.8, 4.9 Hz, 1H), 4.25 - 4.07 (m, 5H), 4.01 (dd, J = 13.3, 2.9 Hz, 1H), 3.84 - 3.70 (m, 2H), 3.70 - 3.60 (m, 2H), 3.55 - 3.49 (m, 2H), 3.36 (s, 3H), 1.19 - 0.89 (m, 30H). 13C NMR (CDC13, 100.6 MHz) δ (ppm): 162.7, 150.6, 137.7, 101.4, 91.2, 81.9, 75.3, 71.9, 69.6, 69.0, 67.4, 60.2, 58.9, 39.5, 17.4, 17.4, 17.3, 17.2, 17.0, 16.9, 16.9, 16.8, 13.4, 12.9, 12.9, 12.5.

1-((6aR, 8R,9S,9aS)-9-hydroxy-2,2, 4, 4-tetraisopropyltetrahydro-6H-furo[3,2-∱][1,3,5,2,4]trioxadisilocin-8-yl)pyrimidine-2, 4(1H,3H)-dione

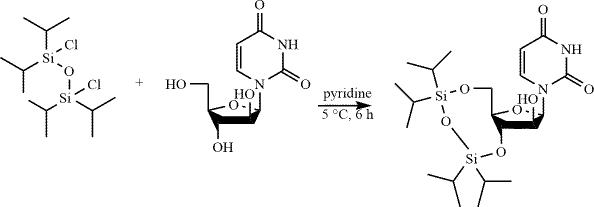

Modified from a procedure outlined by Li and co-workers (E. Li, Y. Wang, W. Yu, Z. Lv, Y. Peng, B. Liu, S. Li, W. Ho, Q. Wang, H. Li, J. Chang, Synthesis and biological evaluation of a novel β-D-2′-deoxy-2′-α-fluoro-2′-β-C-(fluoromethyl)uridine phosphoramidate prodrug for the treatment of hepatitis C virus infection, Eur. J. Med. Chem. 143 (2018) 107-113. https://doi.org/10.1016/j.ejmech.2017.11.024.).

Uridine (2.5 g, 10.24 mmol) was dissolved in pyridine (20 mL). 4.1 mL (12.8 mmol) of 1,3-dichloro-1,1,3,3-tetraisopropyldisiloxane (TIPDSC1) was slowly added at 5° C. The resulting solution was stirred at 5° C. for 6 h. Then, 10 mL of HCl (1 M) was added. The aqueous layer was re-extracted with DCM (3 x 10 mL). The combined organic layers were dried (Na2SO4), filtered and the solvent evaporated. The crude residue was purified by flash chromatography (RP-C18, H2O:MeCN 10-100% 17 CV), which afforded the title compound as a white solid (4.551 g, 91%).

1H NMR (CDC13, 400 MHz) δ (ppm): 9.87 (s, 1H), 7.87 (d, J =8.1 Hz, 1H), 6.11 (d, J = 6.1 Hz, 1H), 5.73 (d, J =8.1 Hz, 1H), 4.67 - 4.52 (m, 1H), 4.25 (d, J =4.9 Hz, 1H), 4.20 - 4.08 (m, 2H), 4.03 (dd, J = 13.4, 2.8 Hz, 1H), 3.79 (dt, J = 9.0, 2.1 Hz, 1H), 1.27 - 0.90 (m, 30H).

### Example 13

Cells were imaged by confocal microscopy after treatment with 200 nM Cy5-labelled E07min aptamer (A-1.0) or 200 nM Cy5-labelled constructs (SQ1-1000-001). For aptamer uptake experiments, 1x10⁴ cells were plated in each well of a µ-Slide 8 well ibiditreat chamber (IBIDI), in 300 µL DMEM containing 10% v/v FBS. For uptake experiments, 3.4x10³ cells (HeLa, MDA-MB-231 and A549 cells) or 6.8x10³ cells (Mcf-7) were plated in each well of a µ-Slide 18 well ibiditreat chamber (IBIDI), in 100 µL DMEM containing 10% v/v FBS. 5 hours after cells had adhered and 48 hours before imaging, media was replaced with 50 particles per cell (PPC) CellLight early endosome-GFP (Thermo Fisher Scientific) for overnight incubation at 37° C. Additionally, 200 µL DMEM aliquots containing 200 nM aptamer were added to cells at corresponding incubation time points. Subsequently, media was removed and replaced with 60 nM LysoTracker Red DND-99 (Thermo Fisher Scientific) in 100-200 µL DMEM for 1 hour at 37° C. Cells were then washed once with Hank’s Balanced Salt Solution (HBSS) (Gibco). Finally, 100-200 µL imaging media (HBSS buffered with 20 mM 4-(2- hydroxyethyl)-1-piperazineethanesulfonic acid (HEPES), adjusted to pH 7.4) was added to cells. Confocal images were captured with an EVOS microscope or a Zeiss LSM510 laser scanning microscope using 60x oil objective lens. Images were produced using ImageJ software.

Confocal data demonstrated that Cy5-labelled constructs with E07min aptamer (SQ1-1000-001) were specifically uptaken by cancer cells expressing EGFR.

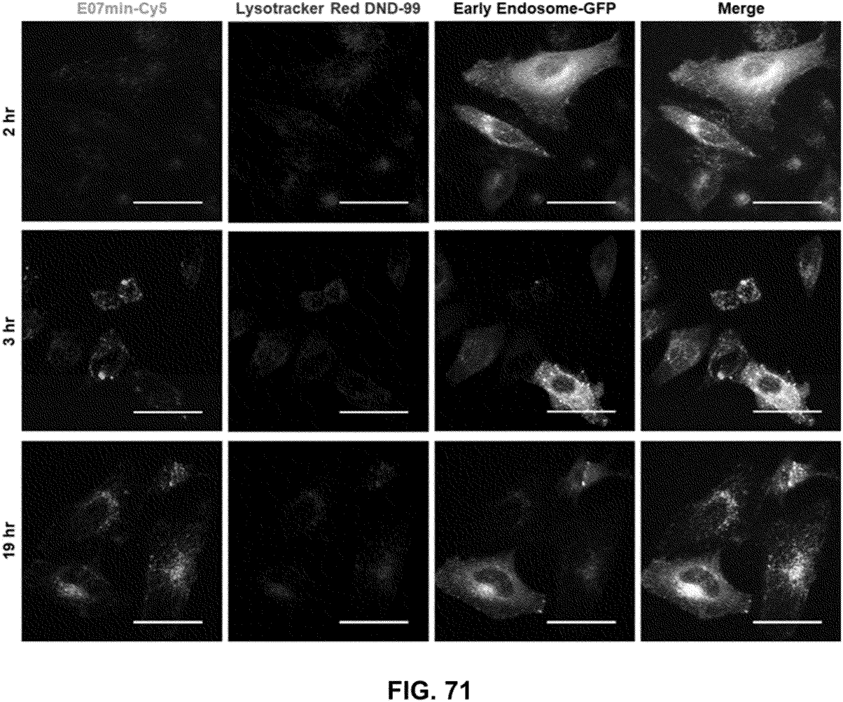

FIG. 71 shows microscopic images of cells following treatment with aptamers. Representative confocal fluorescent microscopy images of live HeLa cells after treatment with E07min aptamer (A-1.0) for increasing lengths of time, at x60 magnification. Lysotracker and early endosome GFP stains were used to identify each compartment. Scale bar 50 µm. The results show that the Aptamers accumulate in lysosomes after internalisation.

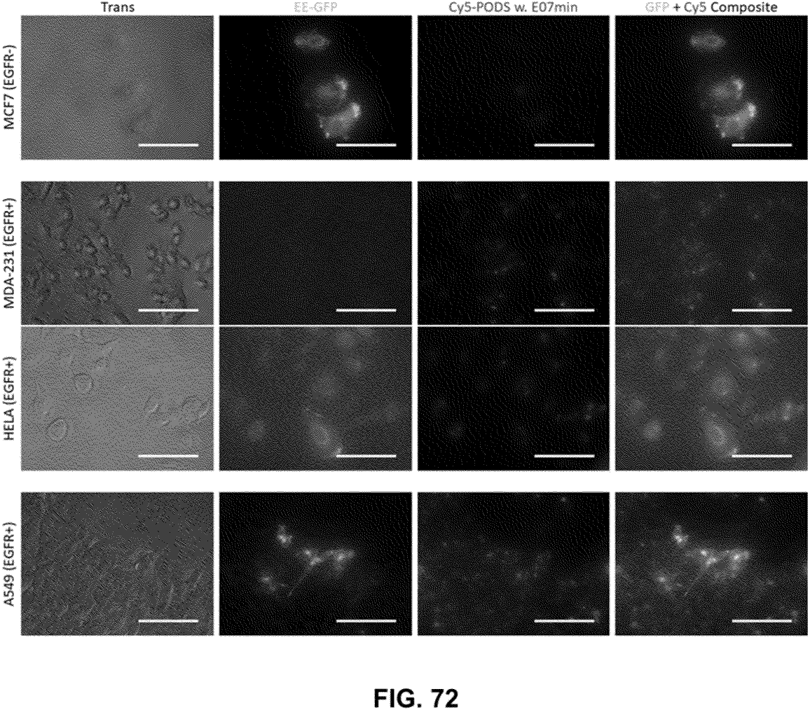

FIG. 72 shows microscopic images of cells following treatment with aptamers. Representative confocal fluorescent microscopy images of different cell types after treatment with Cy5-labelled nanoparticles bearing E07min (SQ1-1000-001) aptamer. Early endosome GFP stain was used to identify colocalization in the early endosomes. Scale bar 100 µm. The results show that Cy5-labelled constructs with E07min aptamer (SQ1-1000-001) are specifically taken up in EGFR+ cells.

### Example 14

Two thiol coupled siRNAs were used to assess the breakage of the disulphide bond upon treatment with different glutathione concentrations. siRNAs at 10 µM concentration were treated with 10 µM and 10 mM glutathione in PBS and incubated at 37° C. Samples at different timepoints were analysed by 15% denaturing PAGE.

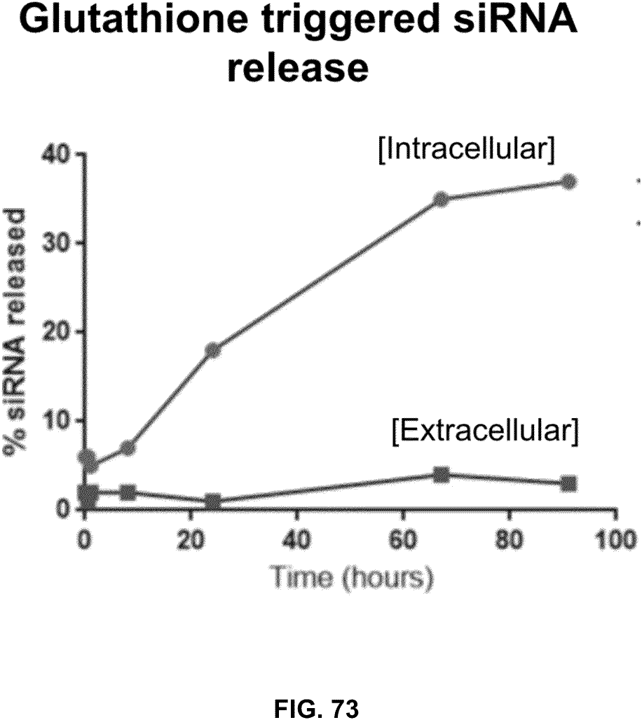

FIG. 73 is a graph of siRNA released in response to glutathione. The amount of released siRNA was quantified by band densitometry on denaturing PAGE. The results show that the disulphide linkage is stable in extracellular concentrations of glutathione (10 µM) but breaks reversibly under intracellular concentrations (10 mM), releasing the attached siRNA.

### Example 15

Several endosomal escape peptides have been synthesized. These sequences are outlined in Table 8.

TABLE 8

peptides synthesized and their estimated isoelectric points (pH(I))

| SEQ ID NO. | Sequence | Estimated isoeletric point (pH(I)) |
|---|---|---|
| 1 | GWWG | 6.0 |
| 2 | CHGWWG | 7.2 |
| 3 | CHGWWGLLL | 7.2 |
| 4 | GWWGLLL | 6.0 |
| 5 | CGWWGLLL | 5.3 |
| 6 | HCGWWGLLL | 7.2 |
| 7 | HGWWGLLL | 7.8 |
| 8 | CGWWG | 5.3 |
| 9 | HGWWG | 7.8 |
| 10 | CGFWFGLLL | 5.3 |
| 11 | GFWFGLLL | 6.0 |
| 12 | HCGFWFGLLL | 7.2 |
| 13 | HGFWFGLLL | 7.8 |
| 14 | CGFWFG | 5.3 |
| 15 | HGFWFG | 7.8 |
| 16 | GFWFG | 6.0 |
| 17 | HCGFWFG | 7.2 |
| 18 | HCGWWG | 7.2 |
| 19 | CLLL | 5.3 |
| 20 | LLL | 6.0 |
| 21 | HCLLL | 7.2 |
| 22 | HLL | 7.8 |
| 23 | CGFWFGLLL | 5.3 |
| 24 | HGFWFGLLL | 7.8 |
| 25 | CHGFWFGLLL | 7.2 |
| 26 | HCGFWFGLLL | 6.0 |
| 27 | GFWFGLLL | 6.0 |
| 28 | GFWFG | 6.0 |
| 29 | CGFWFG | 5.3 |
| 30 | CHGFWFG | 7.2 |
| 31 | HCGFWFG | 7.2 |
| 32 | HGFWFG | 7.8 |
| 33 | GWYWMDL | 3.1 |
| 34 | CGWYWMDL | 4.9 |
| 35 | HGWYWMDL | 4.9 |
| 36 | HCGWYWMDL | 4.9 |
| 37 | CHGWYWMDL | 3.1 |
| 38 | CGWYWMDLLL | 3.1 |
| 39 | HCGWYWMDLLL | 4.9 |
| 40 | HGWYWMDLLL | 4.9 |
| 41 | CHGWYWMDLLL | 4.9 |
| 42 | GWYWMDLLL | 3.1 |
| 43 | FFLIPKG | 10.1 |
| 44 | CFFLIPKG | 9.0 |
| 45 | HCFFLIPKG | 9.0 |
| 46 | CHFFLIPKG | 9.0 |
| 47 | HFFLIPKG | 10.1 |
| 48 | FFLIPKGLLL | 10.1 |
| 49 | CFFLIPKGLLL | 9.0 |
| 50 | HCFFLIPKGLLL | 9.0 |
| 51 | CHFFLIPKGLLL | 9.0 |
| 52 | HFFLIPKGLLL | 10.1 |
| 53 | HYF | 7.8 |
| 54 | CHYF | 7.2 |
| 55 | HCHYF | 7.3 |
| 56 | CHHYF | 7.3 |
| 57 | HYFLLL | 7.8 |
| 58 | CHYFLLL | 7.2 |
| 59 | HHYFLLL | 7.9 |
| 60 | HCHYFLLL | 7.3 |

### Example 16

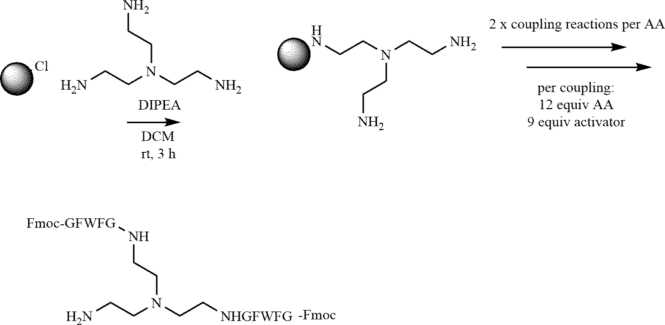

Note: unless otherwise stated, peptide synthesis was carried out under anhydrous conditions.

### Example of Amino Head Group Loading

2-Chlorotrityl chloride resin (80 mg, assuming 1.8 mmol/g) was washed extensively with anhydrous DCM. Tris(2-aminoethyl)amine (693 µL, 8 equiv.) and DIPEA (200 µL, 2 equiv.) in anhydrous DCM (2 mL) was then added and the resultant suspension was agitated at rt for 3 h. The solvent was drained and the resin was washed with DCM (3 x 2 mL) and MeOH (3 x 2 mL). The resin was then agitated with a solution of DCM/MeOH/DIPEA (17/2/1 v/v/v) for 20 min, drained, washed extensively with DCM and then dried under high vacuum.

### Example of Peptide Synthesis

The following solutions were prepared: Deprotection solution: 20% piperidine in DMF; Activator solution: 0.25 M HATU in DMF; Basic solution: 2,6-lutidine (2.05 mL) + DIPEA (1.96 mL) in DMF (5.54 mL) Capping solution: Ac2O (0.92 mL) + 2,6-lutidine (1.3 mL) in DMF (18 mL); Amino acid solution: 0.2 M in DMF.

Pre-loaded amino-based resin (as described above) (50 mg) was swelled in DMF (3 mL) at rt for 30 min. The DMF was then drained and the resin was immersed in 20% piperidine in DMF (this step was repeated). The resin was then washed with DMF (3 x 3 mL), DCM (3 x 3 mL) and again with DMF (3 x 3 mL). In a separate vessel, the desired amino acid solution (1.29 mL), HATU (452 µL, 4.5 equiv.) and base solution (110 µL) were mixed and then added to the resin. The resultant suspension was then agitated at rt for 30 min, the syringe was flushed and the coupling step was repeated. Coupling success was monitored with the Kaiser test. Following successful coupling, the resin was washed with DMF (3 x 3 mL), DCM (3 x 3 mL) and DMF (3 x 3 mL). The resin was then immersed in capping solution (vide supra) for 5 min. The syringe was flushed and the resin was washed with DMF (3 x 3 mL), DCM (3 x 3 mL) and DMF (3 x 3 mL). The process was then repeated (from the deprotection step) until the desired sequence was obtained.

Cleavage from the resin was achieved by submerging it in a mixture of TFA/phenol/water/TIPS (88/5/5/2) and agitating for 3 h, followed by dropwise precipitation into ice cold diethyl ether. The resultant precipitate was then dissolved in acetic acid and lyophilised, affording the desired peptide as the acetate salt.

### Purification

HPLC analysis and purification of the peptides and peptide conjugates was performed on a Thermo Fisher Vanquish Core with a RESOURCETM RPC 3 column (3 mL, 6.4 mm x 100 mm). Samples were dissolved in DMSO and directed injectly. Flow rate 3 mL/min; eluent A: H2O (0.1% TFA); eluent B: MeCN (0.1% TFA).

A gradient of 0-100% B in 15 min was utilised, followed by 100% B for 5 min.

### Linear Sequence GFWFG

Synthesized according to standard peptide synthesis conditions on Rink Amide resin with no headgroup loading step.

HRMS ES+ (m/z): [M]+ calc’d for C33H36N6O6: 612.2696; found: 612.2704.

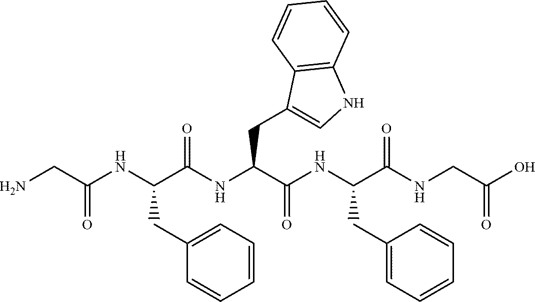 [SMILES: NCC(=O)N[C@@H](Cc1ccccc1)C(=O)N[C@@H](Cc1c[nH]c2ccccc12)C(=O)N[C@@H](Cc1ccccc1)C(=O)NCC(=O)O]

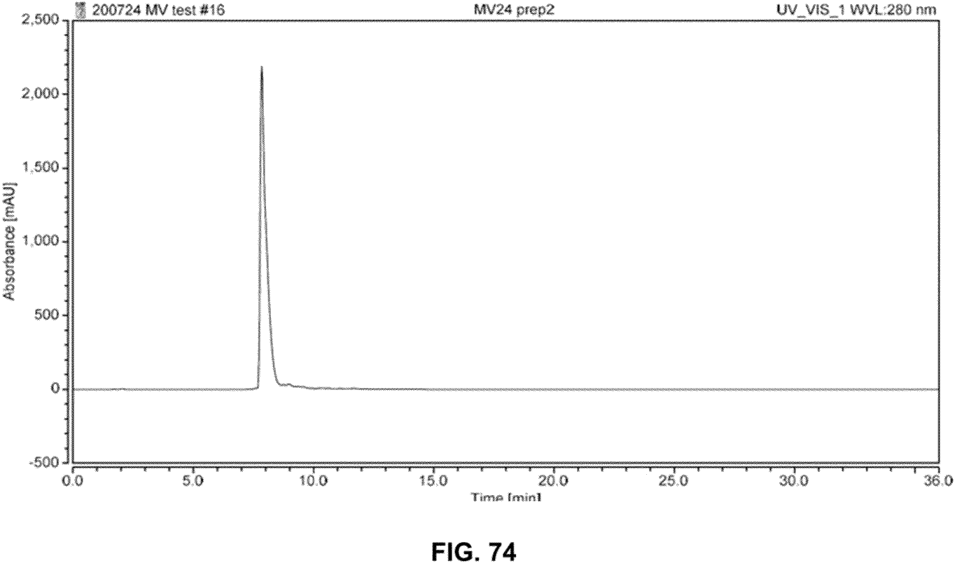

FIG. 74 is a reverse-phase (RP) HPLC trace of GFWFG. HPLC was from 100% H2O to 100% MeCN in 15 min.

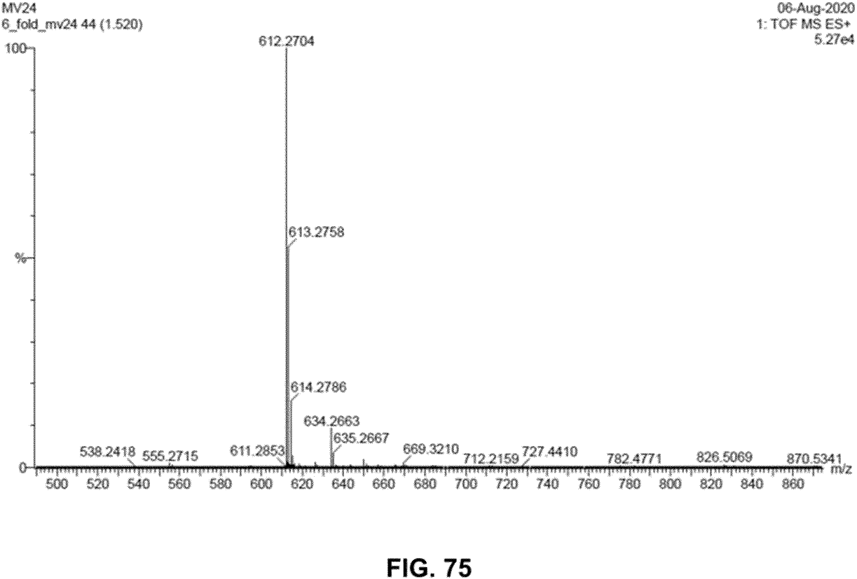

FIG. 75 is a time-of-flight mass spectrometry positive electrospray ionization (TOF MS ES+) graph of GFWFG.

### Maleimidefunctionalized Linear Sequence GFWFG

Synthesized according to standard peptide synthesis conditions on Rink Amide resin with no headgroup loading step. 6-Maleimidohexanoic acid was used in the final coupling.

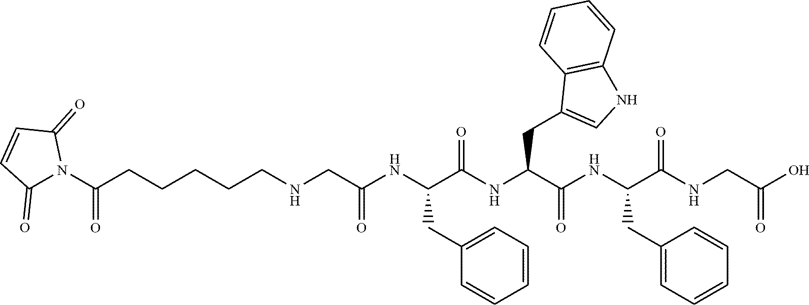 [SMILES: O=C(O)CNC(=O)[C@H](Cc1ccccc1)NC(=O)[C@H](Cc1c[nH]c2ccccc12)NC(=O)[C@H](Cc1ccccc1)NC(=O)CNCCCCCC(=O)N1C(=O)C=CC1=O]

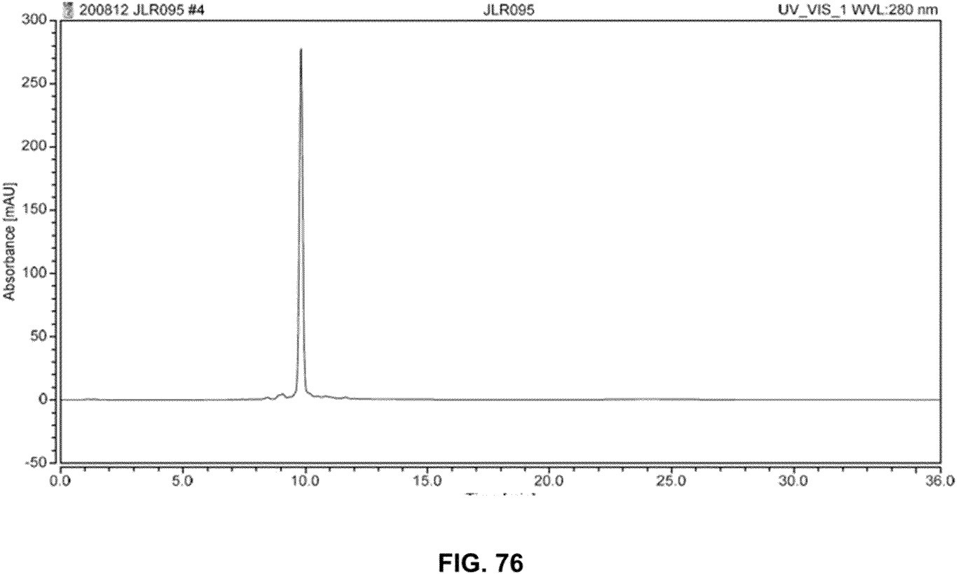

FIG. 76 is a RP HPLC trace of maleimide-modified GFWFG. HPLC was from 100% H₂0 to 100% MeCN in 15 min.

### Branched Sequence GFWFG

Synthesized according to standard peptide synthesis conditions on 2-chlorotrityl chloride resin pre-loaded with tris(2-aminoethyl)amine.

HRMS ES+ (m/z): [M]+ calc’d for C102H107N16014: 1779.8153; found: 1779.8168.

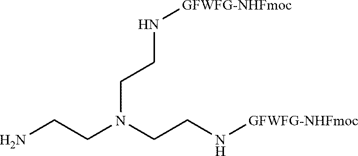

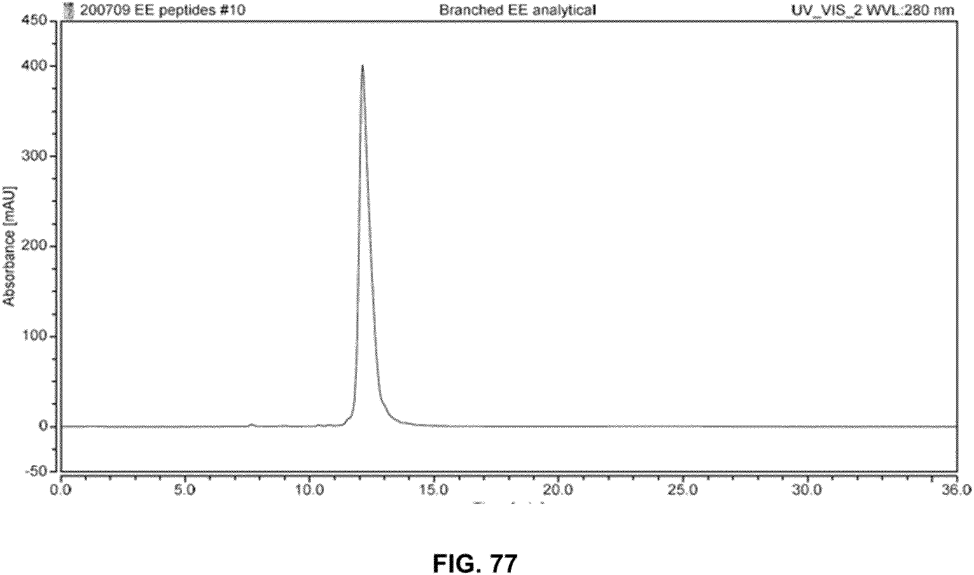

FIG. 77 is a RP HPLC trace of branched GFWFG. HPLC was from 100% H₂0 to 100% MeCN in 15 min.

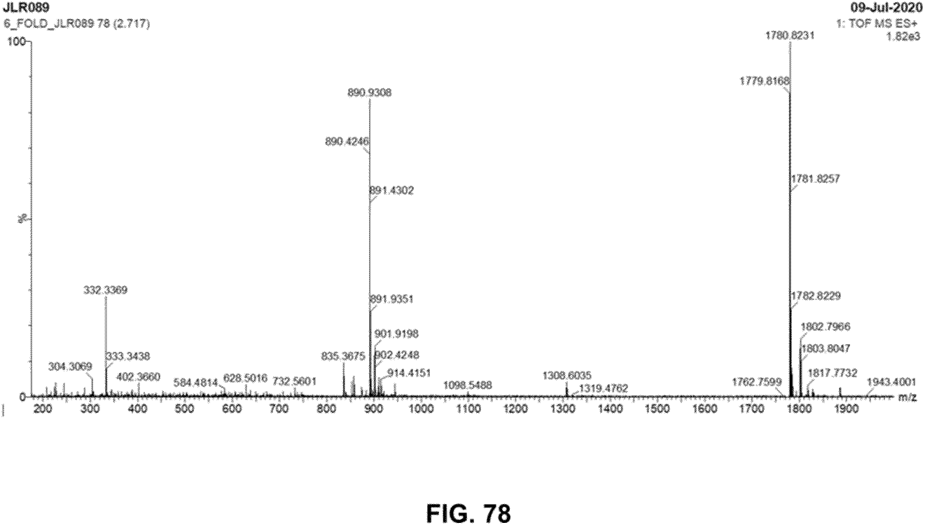

FIG. 78 is a TOF MS ES+ graph of branched GFWFG.

### PEG Maleimidefunctionalized Branched Sequence GFWFG

Peptide was synthesized according to standard peptide synthesis conditions on 2-chlorotrityl chloride resin pre-loaded with tris(2-aminoethyl)amine. Following cleavage from the resin, the Fmoc-protected peptide (3 µM) was reacted with SM(PEG)6 (30 µM) in 12% PBS/DMSO at rt for 16 h, followed by purification by RP HPLC.

$$\text{HRMS ES+} \left(\text{m} / \text{z}\right) : \left[\text{M} / 2\right] \text{+: 1141} \text{.9626} \text{.}$$

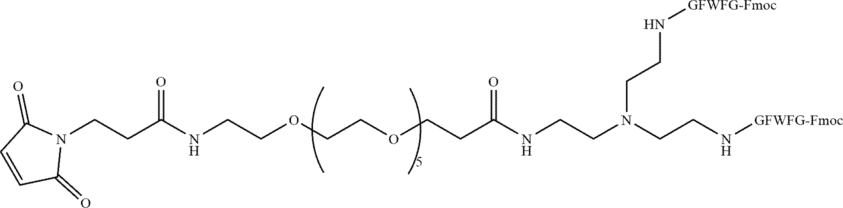

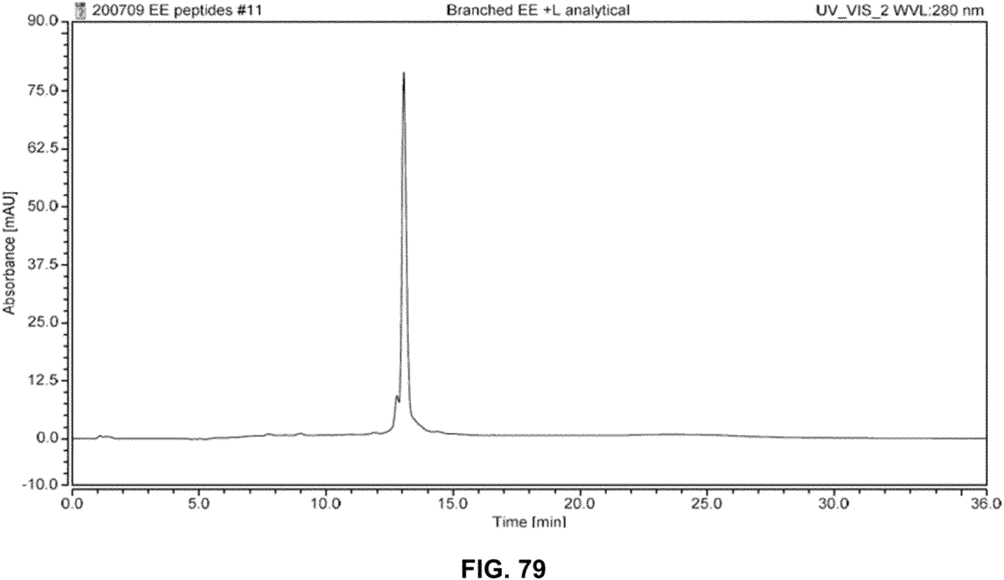

FIG. 79 is a RP HPLC trace of PEG-maleimide branched GFWFG. HPLC was from 100% H₂0 to 100% MeCN in 15 min.

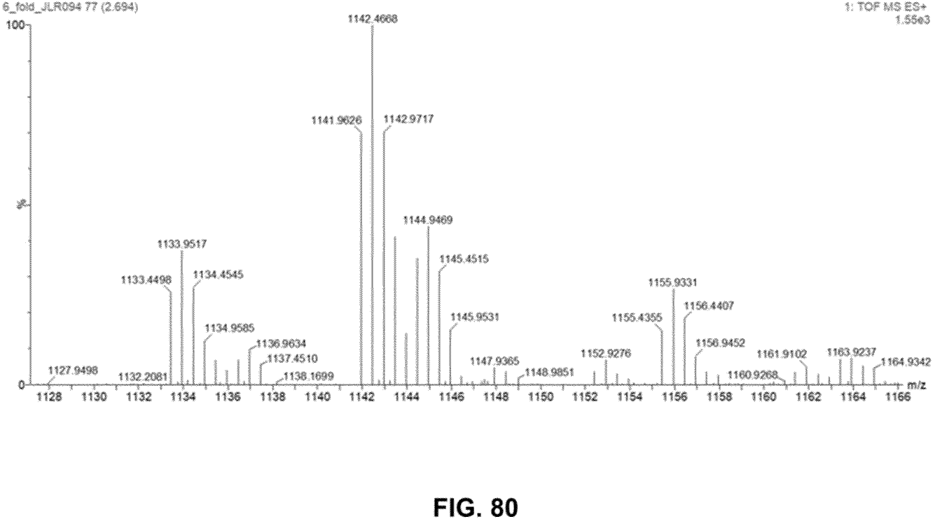

FIG. 80 is a TOF MS ES+ graph of branched GFWFG.

### Example 17

### Fmoc Deprotection with AMA

To ascertain whether the Fmoc protecting groups could be removed post-RNA conjugation, Fmoc-protected branched GFWFG (1 mg) was dissolved in DMSO (10 uL) and was treated with a 1:1 mixture of NH4OH/MeNH2 (AMA). This was then left at rt for 3 h. The AMA was then removed under a stream of nitrogen and the reaction mixture was purified by RP HPLC. Analysis by HRMS confirmed successful deprotection with no observable side reactions.

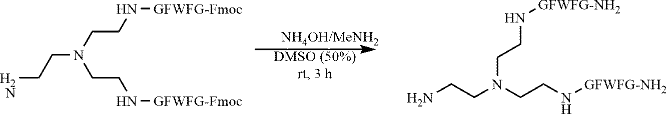

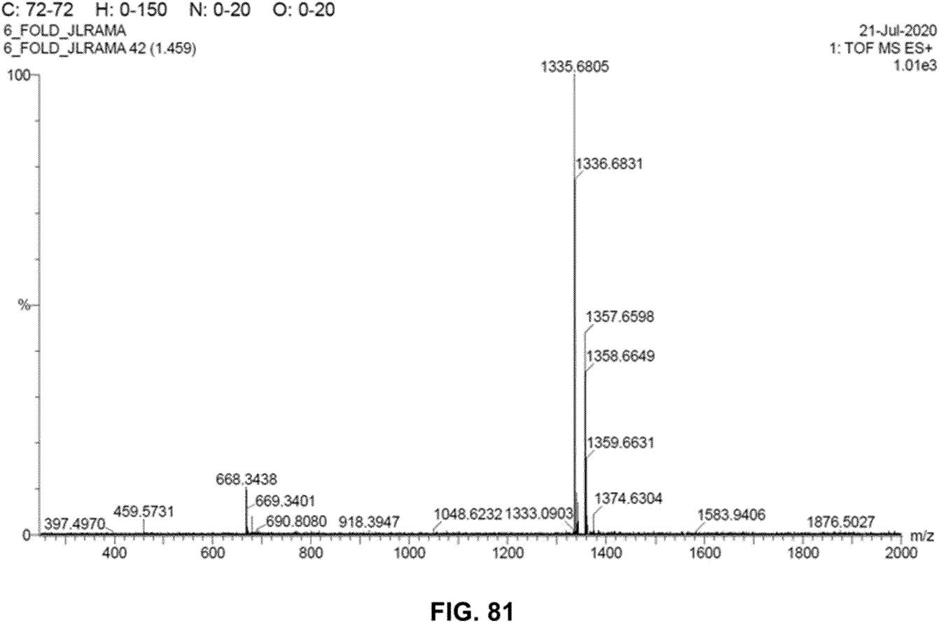

FIG. 81 is a TOF MS ES+ graph of branched GFWFG post AMA-deprotection.

### Example 18

Solution-phase conjugation of maleimide-functionalized peptide to thiol-containing RNA

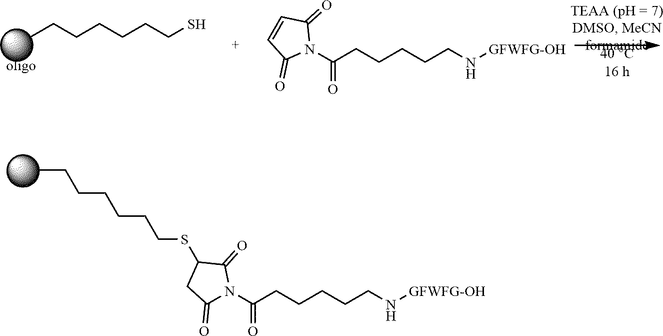

Lyophilized oligonucleotide (234 nmol) was dissolved in water (1 mL) and split into two separate vials. To each vial was added Et3N (10 µL) and 50 µL of a 1 M solution of DTT. The resultant solution was then agitated at rt for 2.5 h. Excess DTT was then removed with a GelPak desalting column (Glen Research) and the RNA concentration was measured. 96 nmol of fully desalted RNA was recovered. The two desalted oligos were pooled together to give a total volume in H2O of 3.6 mL. To this, TEAA (100 mM, pH = 7, 0.8 mL) was added. The peptide (10 equiv. relative to RNA) was dissolved, in a separate vial, in 20% formamide in MeCN to give a concentration of 50 mM. This was added to the freshly desalted oligo solution, which led to noticeable precipitation. DMSO (4.4 mL) was added to give a 1:1 (v/v) mixture of TEAA/DMSO to solubilise the peptide and assist in denaturing of the oligo. The reaction mixture was agitated at rt for 16 h. This can be purified directly by RP HPLC as the difference in RNA retention time vs. RNA-peptide conjugate is approximately 20 min.

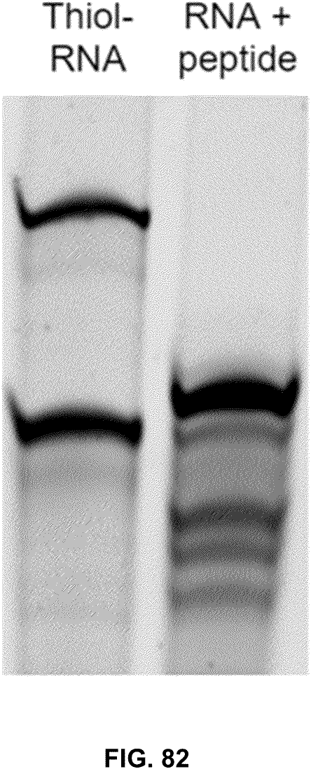

FIG. 82 shows the results of denaturing PAGE of the thiol-RNA vs. the peptide-conjugated RNA.

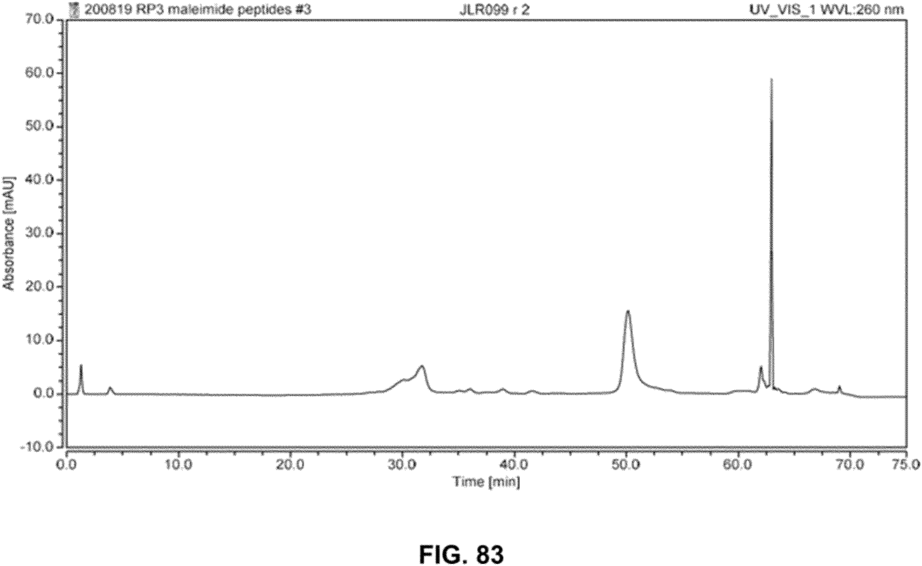

FIG. 83 is a RP HPLC trace of the conjugation reaction mixture. The peak at 30 minutes corresponds to the thiol-RNA. The peak at 50 minutes is the peptide-RNA product.

### Example 19

These couplings were carried out according to a procedure developed by Kye and co-workers (M. Kye, Y. beom Lim, Synthesis and purification of self-assembling peptide-oligonucleotide conjugates by solid-phase peptide fragment condensation, J. Pept. Sci. 24 (2018). https://doi.org/10.1002/psc.3092.).

Briefly, RNAs (1 µmol) were synthesized via the standard phosphoramidite method and modified at the 5′ end with a C6 S-S thiol modifier. This CPG was then divided into the desired molar amount (200 nmol) and treated with DTT (0.25 M in DMF, 100 µL) and DIPEA (2 equiv. relative to DTT) at 37° C. for 3 h. The CPG was then rinsed with DMF (6 x 1 mL) and MeCN (2 x 1 mL) and air-dried. The CPG was then treated with maleimide-modified peptide (400 nmol, 2 equiv.) in DMF (100 µL) with DIPEA (10 equiv.). The reaction mixture was agitated at rt for 16 h, filtered and washed with DMF (3 x 1 mL) and MeCN (3 x 1 mL). The standard RNA deprotection procedure was then performed and the resultant POCs were analyzed via 15% denaturing PAGE.

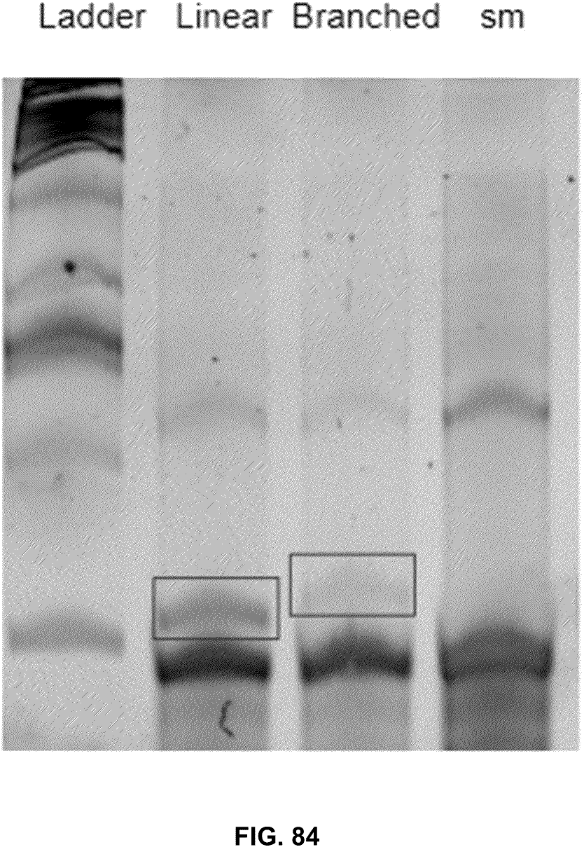

FIG. 84 shows the results of denaturing PAGE (15%) following solid-phase peptide conjugation of thiol-functionalized RNA to maleimide-containing peptides.

### Example 20

Red blood cell hemolysis was used to measure pH-dependent membrane disruption of various compounds at pH values mimicking physiological (7.4), early endosome (6.5) and late endosome/lysosome (5.6).

Briefly, whole human blood was drawn and centrifuged for plasma removal. The remaining erythrocytes were washed three times with 150 mM NaCl and resuspended into phosphate buffers corresponding to the desired pH values. The RBC suspension was then treated with the test compound or one of the controls (i.e. 0.1% Triton-X-100) and the suspension was incubated at 37° C. for 1 h. Intact erythrocytes were pelleted by centrifugation, and the hemoglobin content within the supernatant was measured via absorbance at 541 nm. Percent (%) hemolysis was determined relative to 0.1% Triton-X-100.

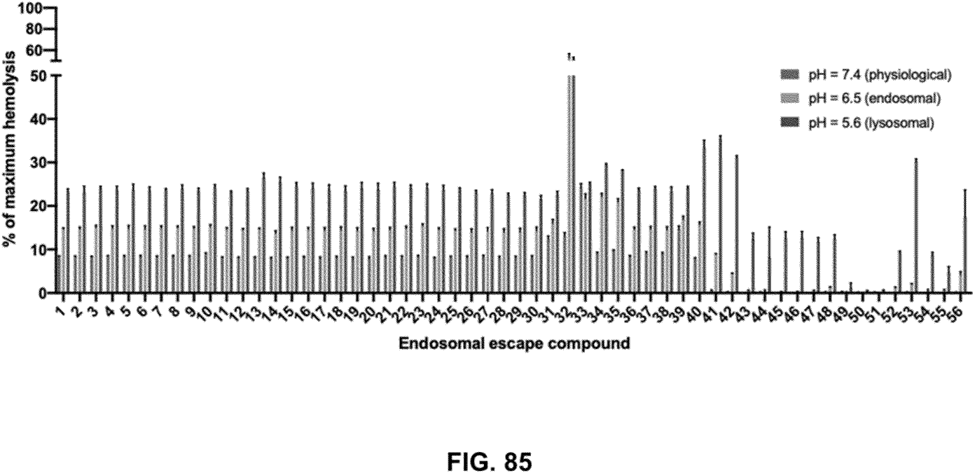

FIG. 85 is a graph of hemolytic activity of a library of potential endosomal escape compounds. % maximum hydrolysis was measured relative to 0.1% Triton-X-100.

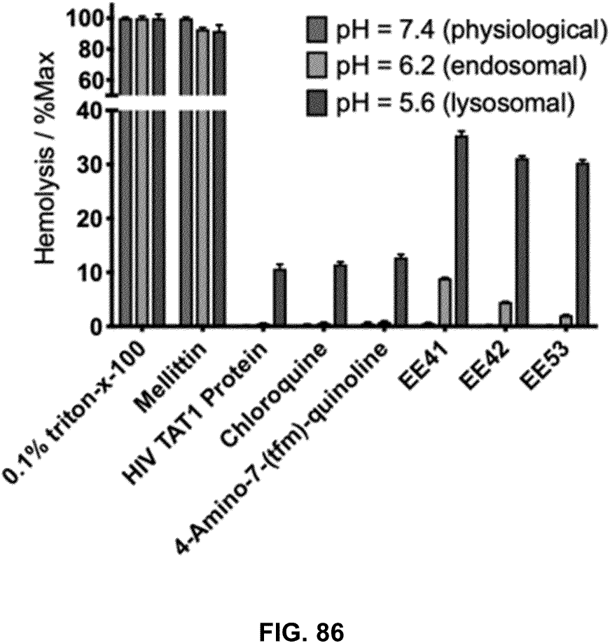

FIG. 86 is a graph of hemolytic activity of most-promising endosomal escape compounds (branched GFWFG peptides with ethylene diamine linker). % maximum hydrolysis was measured relative to 0.1% Triton-X-100.

### Example 21

RNA nanoparticles were assembled, and mixed with Human Serum (HS) and samples were subsequently loaded on gel. We observe the formation of slower mobility bands (shifted to the top of the gel), indicating the formation of larger molecular weight products. This is indicative of the binding of proteins to the nanoparticles.

Native PAGE: To each aliquot of 5 pmol of the assembled construct (assembly in 1x PBS + MgC12 2 mM) was added the desired volume (1 µL for 10%) of Human serum (Sigma) and the final volume made up to 10 µL with PBS buffer for each sample. The solutions were incubated at 37° C. for 30 mins before adding 2 µL of 70% (v/v) glycerine solution to aid loading on a 6% native PAGE gel, 1x TBMg running buffer, and run at 100V for 1 hour. Gel bands were visualized by staining with GelRed™. Mobility shift reveals binding to proteins contained in serum.

Agarose: To each aliquot of 5 pmol of the assembled construct (assembly in 1x PBS + MgC12 2 mM) was added the desired volume (1 µL for 10%) and the final volume made up to 10 µL with PBS buffer for each sample. The solutions were incubated at 37° C. for 30 mins before adding 2 µL of 70% (v/v) glycerine solution to aid loading on a 2.5% native Agarose gel, 1x TBMg running buffer, and run at 100 V for 1 hour.

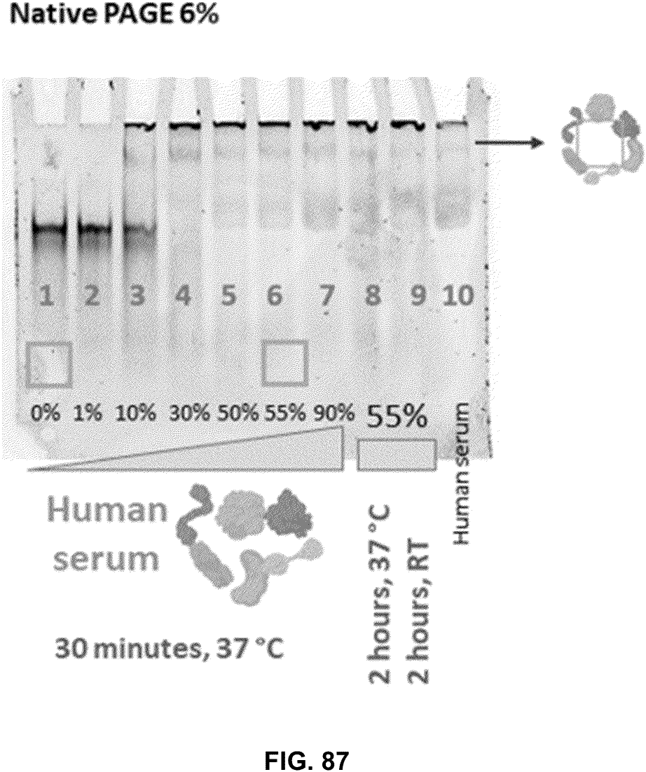

FIG. 87 shows results of native PAGE (6%) to determine protein binding to RNA nanoparticles.

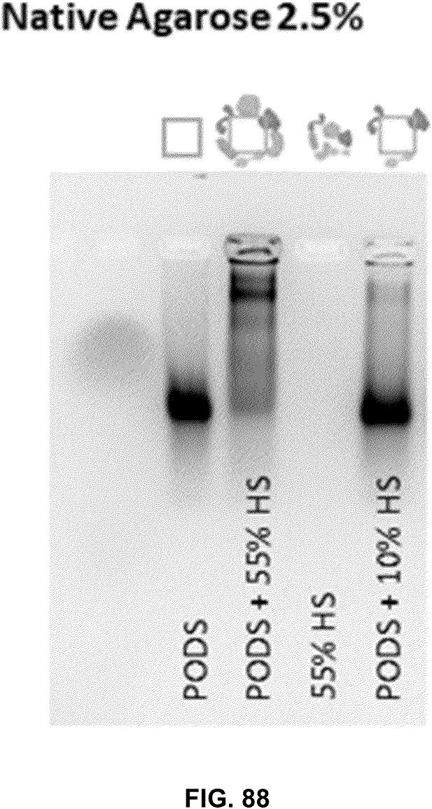

FIG. 88 shows results of native agarose (2.5%) gel to determine protein binding to RNA nanoparticles. TBMg running buffer: 890 mM Tris Borate + 20 mM Mg(OAc)₂, pH 8.3.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1-78. (canceled)

79. A composition comprising:
a hydrophobic moiety; and/or
a hydrophilic moiety
a nucleotide attachment moiety.

80. The composition of claim 79, wherein the nucleotide attachment moiety is selected from the group consisting of a thiol, amine, alkyne, azide, tetrazine, norbomene, dibenzocyclooctyne.

81. The composition of claim 80, wherein the nucleotide attachment moiety is attached to an oligonucleotide.

82. The composition of claim 81, wherein the oligonucleotide is either a 3 to 200 nucleotide RNA or DNA molecule, or a combination thereof.

83. The composition of claim 82, wherein the 2′ position of sugars of the component nucleotides within the oligonucleotide chains are modified with any modifications selected from the group consisting of 2′-O-(2-methoxyethyl) (2′MOE), 2′-methoxy (2′OMe), 2′-fluoro (2′F), 2-′O-acetalesters, 2-guanidinomethyl-2-ethylbutyryloxymethyl (GMEBuOM), 2-amino-2-methylpropionyloxymethyl (AMPrOM), 2-aminomethyl-2-ethylbutyryloxymethyl (AMEBuOM), 2′-O-Pivaloyloxymethyl (PivOM), 2′ amino locked nucleic acids (LNA) modified with amines or peptides mentioned above, 2′-O-[N,N-dimethylamino)ethoxy]ethyl, 2′-N-[N,N-dimethylamino)ethoxy]ethyl, 2′-N-imidazolacetyamide, 2′-O-[3-(guanidinium)propyl], 2′-N [3-(guanidinium)propyl], 2′-O-[3-(guanidinium)ethyl], 2′-N-[3-(guanidinium)ethyl], 2′-0-(N-(aminoethyl)carbamoyl)methyl, 2′-N-(N-(aminoethyl)carbamoyl)methyl, 2′-O-[A-(2-((2-aminoethyl)amino)ethyl)]acetamide, 2′-N-[N-(2-((2-aminoethyl)amino)ethyl)]acetamide, 2′-N-2,2,3,3,4,4,5,5,6,6,7,7,8,8,8-pentadecafluorooctanamide, 2′-N-imidazolacetamide, 2′-O-imidazole methyl, 2′-N-guanidylbenzylamide, and 4′-C-guanidinincarbohydrazidomethyl, 2′-O-imidazolemethyl, 2′-N-imidazolemethylamine ethyl.

84. The composition of claim 82, wherein the oligonucleotide is self-assembled into a nanoparticle, said nanoparticle comprising at least four oligonucleotide strands of 3 to 200 nucleotides in length.

85. The composition of claim 84, wherein the nanoparticle is conjugated to one or more cargo molecules.

86. The composition of claim 85, wherein the cargo molecule is selected from the group consisting of an mRNA molecule, a lnRNA molecule, a miRNA molecule, an siRNA molecule, a shRNA molecule, a ASO molecule, a peptide, a polypeptide, a protein, an antibody, a label, a reporter, a stabilizing agent, a targeting moiety, and a therapeutic agent.

87. The composition of claim 82, wherein the nucleotide attachment moiety is conjugated to a nucleic acid in the oligonucleotide via a linkage to either a 2′ position of a sugar in the nucleic acid, a base in the nucleic acid, or at the 3′ or 5′ end of a component oligonucleotide.

88. The composition of claim 84, wherein the nucleotide attachment moiety is attached to a component oligonucleotide strand within the nanoparticle.

89. The composition of claim 86, wherein the nucleotide attachment moiety is attached to the cargo molecule.

90. The composition of claim 84, wherein the nucleic acid nanoparticle comprises a nucleic acid having at least one linkage selected from the group consisting of a 3-(2-nitrophenyl)-propyl phosphoramidite linkage, a 3-phenylpropyl phosphoramidite linkage, a alkyl phosphorothioate linkage, a aminobutyl phosphoramidite linkage, a aryl phosphorothioate linkage, a dimethylamino phopsphoramidite linkage, a guanidinobutylphosphoramidate linkage, and a phosphorothioate linkage.

91. The composition of claim 79, wherein the hydrophobic moiety promotes escape of the cargo molecule from an endosome within a cell of a subject.

92. The composition of claim 79, wherein the hydrophilic moiety promotes escape of the cargo molecule from an endosome within a cell of a subject.

93. The composition of claim 79, wherein the hydrophilic moiety comprises an amine.

94. The composition of claim 93, wherein the hydrophilic moiety is selected from the group consisting of spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(Hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, imidazole, guanidyl and a β-aminoester.

95. The composition of claim 79, wherein the hydrophilic group is positively-charged at a pH of about 7.0.

96. The composition of claim 79, wherein the hydrophobic moiety is selected from the group consisting of cholesterol, cholesteryl-TEG, tocopherol, methyl, ethyl, butyl, hexyl, octyl, dodecyl, hexadecyl, octadecyl, and 2-methoxyethyl.

97. The composition of claim 79, wherein the hydrophobic component comprises a peptide.

98. The composition of claim 97, wherein the peptide is attached via a linkage selected from the group consisting of a thiol-maleimide linkage, an amide linkage, a disulfide linkage, and a triazole linkage.


SEQUENCE LISTING (92 sequences)

SEQ ID NO: 1 - protein, 4 aa, Artificial Sequence
  synthetic
GWWG

SEQ ID NO: 2 - protein, 6 aa, Artificial Sequence
  synthetic
CHGWWG

SEQ ID NO: 3 - protein, 9 aa, Artificial Sequence
  synthetic
CHGWWGLLL

SEQ ID NO: 4 - protein, 7 aa, Artificial Sequence
  synthetic
GWWGLLL

SEQ ID NO: 5 - protein, 8 aa, Artificial Sequence
  synthetic
CGWWGLLL

SEQ ID NO: 6 - protein, 9 aa, Artificial Sequence
  synthetic
HCGWWGLLL

SEQ ID NO: 7 - protein, 8 aa, Artificial Sequence
  synthetic
HGWWGLLL

SEQ ID NO: 8 - protein, 5 aa, Artificial Sequence
  synthetic
CGWWG

SEQ ID NO: 9 - protein, 5 aa, Artificial Sequence
  synthetic
HGWWG

SEQ ID NO: 10 - protein, 9 aa, Artificial Sequence
  synthetic
CGFWFGLLL

SEQ ID NO: 11 - protein, 8 aa, Artificial Sequence
  synthetic
GFWFGLLL

SEQ ID NO: 12 - protein, 10 aa, Artificial Sequence
  synthetic
HCGFWFGLLL

SEQ ID NO: 13 - protein, 9 aa, Artificial Sequence
  synthetic
HGFWFGLLL

SEQ ID NO: 14 - protein, 6 aa, Artificial Sequence
  synthetic
CGFWFG

SEQ ID NO: 15 - protein, 6 aa, Artificial Sequence
  synthetic
HGFWFG

SEQ ID NO: 16 - protein, 5 aa, Artificial Sequence
  synthetic
GFWFG

SEQ ID NO: 17 - protein, 7 aa, Artificial Sequence
  synthetic
HCGFWFG

SEQ ID NO: 18 - protein, 6 aa, Artificial Sequence
  synthetic
HCGWWG

SEQ ID NO: 19 - protein, 4 aa, Artificial Sequence
  synthetic
CLLL

SEQ ID NO: 20 - protein, 5 aa, Artificial Sequence
  synthetic
HCLLL

SEQ ID NO: 21 - protein, 9 aa, Artificial Sequence
  synthetic
CGFWFGLLL

SEQ ID NO: 22 - protein, 9 aa, Artificial Sequence
  synthetic
HGFWFGLLL

SEQ ID NO: 23 - protein, 10 aa, Artificial Sequence
  synthetic
CHGFWFGLLL

SEQ ID NO: 24 - protein, 10 aa, Artificial Sequence
  synthetic
HCGFWFGLLL

SEQ ID NO: 25 - protein, 8 aa, Artificial Sequence
  synthetic
GFWFGLLL

SEQ ID NO: 26 - protein, 5 aa, Artificial Sequence
  synthetic
GFWFG

SEQ ID NO: 27 - protein, 6 aa, Artificial Sequence
  synthetic
CGFWFG

SEQ ID NO: 28 - protein, 7 aa, Artificial Sequence
  synthetic
CHGFWFG

SEQ ID NO: 29 - protein, 7 aa, Artificial Sequence
  synthetic
HCGFWFG

SEQ ID NO: 30 - protein, 6 aa, Artificial Sequence
  synthetic
HGFWFG

SEQ ID NO: 31 - protein, 7 aa, Artificial Sequence
  synthetic
GWYWMDL

SEQ ID NO: 32 - protein, 8 aa, Artificial Sequence
  synthetic
CGWYWMDL

SEQ ID NO: 33 - protein, 8 aa, Artificial Sequence
  synthetic
HGWYWMDL

SEQ ID NO: 34 - protein, 9 aa, Artificial Sequence
  synthetic
HCGWYWMDL

SEQ ID NO: 35 - protein, 9 aa, Artificial Sequence
  synthetic
CHGWYWMDL

SEQ ID NO: 36 - protein, 10 aa, Artificial Sequence
  synthetic
CGWYWMDLLL

SEQ ID NO: 37 - protein, 11 aa, Artificial Sequence
  synthetic
HCGWYWMDLLL

SEQ ID NO: 38 - protein, 10 aa, Artificial Sequence
  synthetic
HGWYWMDLLL

SEQ ID NO: 39 - protein, 11 aa, Artificial Sequence
  synthetic
CHGWYWMDLLL

SEQ ID NO: 40 - protein, 9 aa, Artificial Sequence
  synthetic
GWYWMDLLL

SEQ ID NO: 41 - protein, 7 aa, Artificial Sequence
  synthetic
FFLIPKG

SEQ ID NO: 42 - protein, 8 aa, Artificial Sequence
  synthetic
CFFLIPKG

SEQ ID NO: 43 - protein, 9 aa, Artificial Sequence
  synthetic
HCFFLIPKG

SEQ ID NO: 44 - protein, 9 aa, Artificial Sequence
  synthetic
CHFFLIPKG

SEQ ID NO: 45 - protein, 8 aa, Artificial Sequence
  synthetic
HFFLIPKG

SEQ ID NO: 46 - protein, 10 aa, Artificial Sequence
  synthetic
FFLIPKGLLL

SEQ ID NO: 47 - protein, 11 aa, Artificial Sequence
  synthetic
CFFLIPKGLLL

SEQ ID NO: 48 - protein, 12 aa, Artificial Sequence
  synthetic
HCFFLIPKGLLL

SEQ ID NO: 49 - protein, 12 aa, Artificial Sequence
  synthetic
CHFFLIPKGLLL

SEQ ID NO: 50 - protein, 11 aa, Artificial Sequence
  synthetic
HFFLIPKGLLL

SEQ ID NO: 51 - protein, 4 aa, Artificial Sequence
  synthetic
CHYF

SEQ ID NO: 52 - protein, 5 aa, Artificial Sequence
  synthetic
HCHYF

SEQ ID NO: 53 - protein, 5 aa, Artificial Sequence
  synthetic
CHHYF

SEQ ID NO: 54 - protein, 6 aa, Artificial Sequence
  synthetic
HYFLLL

SEQ ID NO: 55 - protein, 7 aa, Artificial Sequence
  synthetic
CHYFLLL

SEQ ID NO: 56 - protein, 7 aa, Artificial Sequence
  synthetic
HHYFLLL

SEQ ID NO: 57 - protein, 8 aa, Artificial Sequence
  synthetic
HCHYFLLL

SEQ ID NO: 58 - RNA, 47 nt, Artificial Sequence
  synthetic
  misc_feature (7)..(10): 2' Fluorine
  misc_feature (12)..(13): 2' Fluorine
  misc_feature (15)..(15): 2' Fluorine
  misc_feature (20)..(20): 2' Fluorine
  misc_feature (23)..(24): 2' Fluorine
  misc_feature (28)..(29): 2' Fluorine
  misc_feature (31)..(33): 2' Fluorine
  misc_feature (36)..(37): 2' Fluorine
  misc_feature (39)..(47): 2' Fluorine
gggaaacucugucgugggacggucagacuguucaaccacuccucuuc

SEQ ID NO: 59 - RNA, 47 nt, Artificial Sequence; identical to SEQ ID NO: 58
  synthetic
  misc_feature (1)..(1): 5' thiol
  misc_feature (7)..(10): 2' Fluorine
  misc_feature (12)..(13): 2' Fluorine
  misc_feature (15)..(15): 2' Fluorine
  misc_feature (20)..(20): 2' Fluorine
  misc_feature (23)..(24): 2' Fluorine
  misc_feature (28)..(29): 2' Fluorine
  misc_feature (31)..(33): 2' Fluorine
  misc_feature (36)..(37): 2' Fluorine
  misc_feature (39)..(47): 2' Fluorine

SEQ ID NO: 60 - RNA, 47 nt, Artificial Sequence
  synthetic
  misc_feature (16)..(16): 2' F
  misc_feature (20)..(20): 2' F
  misc_feature (23)..(23): 2' F
  misc_feature (25)..(26): 2' F
  misc_feature (28)..(28): 2' F
  misc_feature (30)..(33): 2' F
  misc_feature (36)..(38): 2' F
  misc_feature (40)..(44): 2' F
  misc_feature (47)..(47): 2' F
gggaaagaagaggaguggacgguacuguguuucaaccugucucugac

SEQ ID NO: 61 - RNA, 47 nt, Artificial Sequence; identical to SEQ ID NO: 60
  synthetic
  misc_feature (1)..(1): 5' thiol
  misc_feature (16)..(16): 2' Fluorine
  misc_feature (20)..(20): 2' Fluorine
  misc_feature (23)..(23): 2' Fluorine
  misc_feature (25)..(26): 2' Fluorine
  misc_feature (28)..(28): 2' Fluorine
  misc_feature (30)..(33): 2' Fluorine
  misc_feature (36)..(37): 2' Fluorine
  misc_feature (40)..(44): 2' Fluorine
  misc_feature (47)..(47): 2' Fluorine

SEQ ID NO: 62 - RNA, 47 nt, Artificial Sequence
  synthetic
  misc_feature (8)..(8): 2' Fluorine
  misc_feature (11)..(11): 2' Fluorine
  misc_feature (13)..(13): 2' Fluorine
  misc_feature (16)..(16): 2' Fluorine
  misc_feature (20)..(20): 2' Fluorine
  misc_feature (23)..(23): 2' Fluorine
  misc_feature (25)..(25): 2' Fluorine
  misc_feature (27)..(28): 2' Fluorine
  misc_feature (36)..(38): 2' Fluorine
  misc_feature (40)..(40): 2' Fluorine
  misc_feature (43)..(43): 2' Fluorine
gggaaagcaguguagcggacggugugucaguucaacccacgacagag

SEQ ID NO: 63 - RNA, 67 nt, Artificial Sequence
  synthetic
  misc_feature (1)..(1): 2' Fluorine
  misc_feature (4)..(4): 2' Fluorine
  misc_feature (6)..(8): 2' Fluorine
  misc_feature (11)..(11): 2' Fluorine
  misc_feature (13)..(13): 2' Fluorine
  misc_feature (15)..(15): 2' Fluorine
  misc_feature (17)..(19): 2' Fluorine
  misc_feature (28)..(28): 2' Fluorine
  misc_feature (31)..(31): 2' Fluorine
  misc_feature (40)..(40): 2' Fluorine
  misc_feature (43)..(43): 2' Fluorine
  misc_feature (45)..(45): 2' Fluorine
  misc_feature (46)..(47): 2' Fluorine
  misc_feature (50)..(52): 2' Fluorine
  misc_feature (55)..(57): 2' Fluorine
  misc_feature (58)..(58): 2' Fluorine
  misc_feature (59)..(59): 2' Fluorine
  misc_feature (62)..(62): 2' Fluorine
caguguccgauauacgcucggggaaagcaguguagcggacggugugucaguucaacccacgacagag

SEQ ID NO: 64 - RNA, 47 nt, Artificial Sequence
  synthetic
  misc_feature (8)..(9): 2' Fluorine
  misc_feature (15)..(15): 2' Fluorine
  misc_feature (20)..(20): 2' Fluorine
  misc_feature (23)..(25): 2' Fluorine
  misc_feature (29)..(33): 2' Fluorine
  misc_feature (36)..(37): 2' Fluorine
  misc_feature (39)..(40): 2' Fluorine
  misc_feature (42)..(42): 2' Fluorine
  misc_feature (44)..(45): 2' Fluorine
  misc_feature (47)..(47): 2' Fluorine
gggaaagucagagacaggacggucuaggucuucaaccgcuacacugc

SEQ ID NO: 65 - RNA, 58 nt, Artificial Sequence
  synthetic
  misc_feature (1)..(1): 5' thiol
  misc_feature (8)..(9): 2' Fluorine
  misc_feature (15)..(15): 2' Fluorine
  misc_feature (20)..(20): 2' Fluorine
  misc_feature (23)..(25): 2' Fluorine
  misc_feature (29)..(33): 2' Fluorine
  misc_feature (36)..(37): 2' Fluorine
  misc_feature (39)..(40): 2' Fluorine
  misc_feature (42)..(42): 2' Fluorine
  misc_feature (44)..(45): 2' Fluorine
  misc_feature (47)..(47): 2' Fluorine
gggaaacuagauuggaacacaguauuggacagucugauuggacugacacauuggagac

SEQ ID NO: 66 - RNA, 58 nt, Artificial Sequence; identical to SEQ ID NO: 65
  synthetic
  misc_feature (7)..(8): 2' Fluorine
  misc_feature (12)..(13): 2' Fluorine
  misc_feature (18)..(18): 2' Fluorine
  misc_feature (20)..(20): 2' Fluorine
  misc_feature (23)..(23): 2' Fluorine
  misc_feature (25)..(26): 2' Fluorine
  misc_feature (33)..(35): 2' Fluorine
  misc_feature (38)..(39): 2' Fluorine
  misc_feature (43)..(44): 2' Fluorine
  misc_feature (47)..(47): 2' Fluorine
  misc_feature (49)..(49): 2' Fluorine
  misc_feature (51)..(52): 2' Fluorine
  misc_feature (58)..(58): 2' Fluorine

SEQ ID NO: 67 - RNA, 58 nt, Artificial Sequence; identical to SEQ ID NO: 65
  synthetic
  misc_feature (1)..(1): 5' Cy5
  misc_feature (7)..(8): 2' Fluorine
  misc_feature (12)..(13): 2' Fluorine
  misc_feature (18)..(18): 2' Fluorine
  misc_feature (20)..(20): 2' Fluorine
  misc_feature (23)..(23): 2' Fluorine
  misc_feature (25)..(26): 2' Fluorine
  misc_feature (30)..(30): 2' Fluorine
  misc_feature (33)..(35): 2' Fluorine
  misc_feature (38)..(39): 2' Fluorine
  misc_feature (43)..(44): 2' Fluorine
  misc_feature (47)..(47): 2' Fluorine
  misc_feature (49)..(49): 2' Fluorine
  misc_feature (51)..(52): 2' Fluorine
  misc_feature (58)..(58): 2' Fluorine
  misc_feature (58)..(58): 2' Fluorine

SEQ ID NO: 68 - RNA, 58 nt, Artificial Sequence; identical to SEQ ID NO: 65
  synthetic
  misc_feature (1)..(1): 5' Cy5
  misc_feature (7)..(8): 2' Fluorine
  misc_feature (12)..(13): 2' Fluorine
  misc_feature (18)..(18): 2' Fluorine
  misc_feature (20)..(20): 2' Fluorine
  misc_feature (23)..(23): 2' Fluorine
  misc_feature (25)..(26): 2' Fluorine
  misc_feature (30)..(30): 2' Fluorine
  misc_feature (33)..(35): 2' Fluorine
  misc_feature (38)..(39): 2' Fluorine
  misc_feature (43)..(44): 2' Fluorine
  misc_feature (47)..(47): 2' Fluorine
  misc_feature (49)..(49): 2' Fluorine
  misc_feature (51)..(52): 2' Fluorine
  misc_feature (58)..(58): 2' Fluorine

SEQ ID NO: 69 - DNA, 21 bp, Artificial Sequence
  synthetic
gcaauuacaugagcgagcatt

SEQ ID NO: 70 - DNA, 21 bp, Artificial Sequence
  synthetic
  misc_feature (1)..(1): 5' thiol
  misc_feature (2)..(2): 2' Fluorine
  misc_feature (2)..(2): 2' Fluorine
  misc_feature (5)..(6): 2' Fluorine
  misc_feature (8)..(8): 2' Fluorine
  misc_feature (14)..(14): 2' Fluorine
  misc_feature (18)..(18): 2' Fluorine
gcaauuacaugagcgagcatt

SEQ ID NO: 71 - RNA, 21 nt, Artificial Sequence
  synthetic
ugcucgcucauguaauugcgg

SEQ ID NO: 72 - DNA, 21 bp, Artificial Sequence
  synthetic
  misc_feature (1)..(1): 2' Fluorine
  misc_feature (3)..(5): 2' Fluorine
  misc_feature (7)..(9): 2' Fluorine
  misc_feature (11)..(11): 2' Fluorine
  misc_feature (13)..(13): 2' Fluorine
  misc_feature (16)..(17): 2' Fluorine
  misc_feature (19)..(19): 2' Fluorine
ugcucgcucauguaauugcgg

SEQ ID NO: 73 - DNA, 25 bp, Artificial Sequence
  synthetic
gcaauuacaugagcgagcacuugtt

SEQ ID NO: 74 - RNA, 27 nt, Artificial Sequence
  synthetic
aacaagugcucgcucauguaauugcgg

SEQ ID NO: 75 - DNA, 25 bp, Artificial Sequence
  synthetic
  misc_feature (1)..(1): 5' phosphate
  misc_feature (4)..(4): 2'-O-methylation
  misc_feature (4)..(4): 2'-O-methylation
  misc_feature (10)..(10): 2'-O-methylation
  misc_feature (14)..(14): 2'-O-methylation
  misc_feature (18)..(18): 2'-O-methylation
agaucacccuccuuaaauauuaatt

SEQ ID NO: 76 - DNA, 27 bp, Artificial Sequence
  synthetic
  misc_feature (1)..(4): 2'-O-methylation
  misc_feature (14)..(14): 2'-O-methylation
  misc_feature (23)..(23): 2'-O-methylation
aauuaauauuuaaggagggugaucutt

SEQ ID NO: 77 - RNA, 48 nt, Artificial Sequence
  synthetic
  misc_feature (1)..(1): 5' Cy5
  misc_feature (4)..(4): 2' Fluorine
  misc_feature (8)..(10): 2' Fluorine
  misc_feature (13)..(14): 2' Fluorine
  misc_feature (16)..(17): 2' Fluorine
  misc_feature (19)..(19): 2' Fluorine
  misc_feature (29)..(29): 2' Fluorine
  misc_feature (31)..(32): 2' Fluorine
  misc_feature (37)..(38): 2' Fluorine
  misc_feature (43)..(44): 2' Fluorine
  misc_feature (46)..(48): 2' Fluorine
ggacggauuuaaucgccguagaaaagcaugucaaagccggaaccgucc

SEQ ID NO: 78 - RNA, 74 nt, Artificial Sequence
  synthetic
  misc_feature (1)..(1): 2' Fluorine
  misc_feature (5)..(5): 2' Fluorine
  misc_feature (7)..(7): 2' Fluorine
  misc_feature (9)..(9): 2' Fluorine
  misc_feature (11)..(12): 2' Fluorine
  misc_feature (16)..(16): 2' Fluorine
  misc_feature (18)..(19): 2' Fluorine
  misc_feature (21)..(26): 2' Fluorine
  misc_feature (30)..(30): 2' Fluorine
  misc_feature (34)..(36): 2' Fluorine
  misc_feature (39)..(40): 2' Fluorine
  misc_feature (42)..(43): 2' Fluorine
  misc_feature (45)..(45): 2' Fluorine
  misc_feature (53)..(53): 2' Fluorine
  misc_feature (55)..(55): 2' Fluorine
  misc_feature (57)..(58): 2' Fluorine
  misc_feature (63)..(64): 2' Fluorine
  misc_feature (69)..(70): 2' Fluorine
  misc_feature (72)..(74): 2' Fluorine
cgagcguauaucggacacuguuuuuuggacggauuuaaucgccguagaaaagcaugucaaagccggaaccgucc

SEQ ID NO: 79 - RNA, 39 nt, Artificial Sequence
  synthetic
  misc_feature (1)..(1): 5' Cy5
  misc_feature (2)..(5): 2' fluorine
  misc_feature (8)..(8): 2' fluorine
  misc_feature (11)..(11): 2' fluorine
  misc_feature (13)..(13): 2' fluorine
  misc_feature (15)..(18): 2' fluorine
  misc_feature (21)..(21): 2' fluorine
  misc_feature (23)..(24): 2' fluorine
  misc_feature (27)..(29): 2' fluorine
  misc_feature (32)..(32): 2' fluorine
  misc_feature (39)..(39): 2' fluorine
gccuuaguaacgugcuuugaugucgauucgacaggaggc

SEQ ID NO: 80 - protein, 5 aa, Artificial Sequence
  synthetic
  MISC_FEATURE (1)..(1): NHS-PEG6-Maleimide conjugated to N-terminus
GFWFG

SEQ ID NO: 81 - protein, 5 aa, Artificial Sequence
  synthetic
  MISC_FEATURE: 6-maleimidohexanoic acid conjugated to N-terminus
  MISC_FEATURE (1)..(1): 6-maleimidohexanoic acid conjugated to N-terminus
GFWFG

SEQ ID NO: 82 - protein, 5 aa, Artificial Sequence
  synthetic
  MISC_FEATURE (1)..(1): ethyene diamine conjugated to N-terminus
GFWFG

SEQ ID NO: 83 - protein, 5 aa, Artificial Sequence
  synthetic
  MISC_FEATURE (1)..(1): ethylene diamin conjugated to N-terminus and NHS-PEG6-Maleimide conjugated to branching head group
GFWFG

SEQ ID NO: 84 - DNA, 21 bp, Artificial Sequence
  synthetic
gcaauuacaugagcgagcatt

SEQ ID NO: 85 - RNA, 21 nt, Artificial Sequence
  synthetic
ugcucgcucauguaauugcgg

SEQ ID NO: 86 - DNA, 21 bp, Artificial Sequence
  synthetic
gcaauuacaugagcgagcatt

SEQ ID NO: 87 - RNA, 21 nt, Artificial Sequence
  synthetic
ugcucgcucauguaauugcgg

SEQ ID NO: 88 - DNA, 21 bp, Artificial Sequence
  synthetic
  misc_feature (1)..(1): 5' thiol
  misc_feature (2)..(2): 2' fluorine
  misc_feature (5)..(6): 2' fluorine
  misc_feature (8)..(8): 2' fluorine
  misc_feature (10)..(10): 2' fluorine
  misc_feature (14)..(14): 2' fluorine
  misc_feature (18)..(18): 2' fluorine
gcaauuacaugagcgagcatt

SEQ ID NO: 89 - RNA, 21 nt, Artificial Sequence
  synthetic
  misc_feature (1)..(1): 5' thiol
  misc_feature (1)..(1): 2' fluorine
  misc_feature (3)..(5): 2' fluorine
  misc_feature (7)..(9): 2' fluorine
  misc_feature (11)..(11): 2' fluorine
  misc_feature (13)..(13): 2' fluorine
  misc_feature (16)..(17): 2' fluorine
  misc_feature (19)..(19): 2' fluorine
ugcucgcucauguaauugcgg

SEQ ID NO: 90 - RNA, 47 nt, Artificial Sequence; identical to SEQ ID NO: 58
  synthetic
  misc_feature (1)..(1): 5' thiol
  misc_feature (6)..(10): 2' fluorine
  misc_feature (12)..(13): 2' fluorine
  misc_feature (15)..(15): 2' fluorine
  misc_feature (20)..(20): 2' fluorine
  misc_feature (23)..(24): 2' fluorine
  misc_feature (28)..(29): 2' fluorine
  misc_feature (31)..(33): 2' fluorine
  misc_feature (36)..(37): 2' fluorine
  misc_feature (39)..(46): 2' fluorine

SEQ ID NO: 91 - DNA, 21 bp, Artificial Sequence
  synthetic
  misc_feature (1)..(1): 5' thiol
  misc_feature (2)..(2): 2' fluorinel
  misc_feature (5)..(6): 2' fluorinel
  misc_feature (8)..(8): 2' fluorinel
  misc_feature (10)..(10): 2' fluorinel
  misc_feature (14)..(14): 2' fluorinel
  misc_feature (18)..(18): 2' fluorinel
gcaauuacaugagcgagcatt

SEQ ID NO: 92 - RNA, 21 nt, Artificial Sequence
  synthetic
  misc_feature (1)..(1): 5' thiol
  misc_feature (1)..(1): 2' fluorine
  misc_feature (3)..(5): 2' fluorine
  misc_feature (7)..(9): 2' fluorine
  misc_feature (11)..(11): 2' fluorine
  misc_feature (13)..(13): 2' fluorine
  misc_feature (16)..(17): 2' fluorine
  misc_feature (19)..(19): 2' fluorine
ugcucgcucauguaauugcgg